SEQUENCE LISTING (67 sequences)

SEQ ID NO: 1 - DNA, 2901 bp, Persea americana
atggctttcacactgaaaagcacaccatccccgctctcttcctcctcttcaaccccattccatcagcaagccaagaagaaacctcttctccttccaaaccatcgaccccaccaccttcctcaaccaatctcctgcaatagcagcaacaacagtgaaaaaaatgaaacccaaaaccatggtagaactatcgatagaagaaatgttcttctaggcttgggaggcctctatggcgctgctaccgccttctccatcgacccgaagaaggccgctgcggcaccagttctcccacctgatctctcccaatgtggagcagcagatctcccggctggtgccacccccaccaactgctgcccccccttcacccaaaagattgtggatttcaagctcccctccccctcctcccccatgcgcgttcgccctgccgctcatcttgctgataaagaatacatagccaagtatgagaaggcaattgcactcatgaaggctctcccagctgatgaccccaggaacttcactcaacaggtgaataaattttaagccctacatttcaaaaaaaaaaaaaaaaatggaccctattctgtcttattaattataataagaagcatcacatacaatacataacaaaattgaagtcctgcatttatgcagcaggaggattttactctccgcaactttcatttcaaaagagagagaggaatactgcatttcaactcttagtatacgaaagcatgaccttctttagtctcattttttttcttatgttttgcaccaaacgaaccaaaacagaggttgaatagaaaatgagaacaattttcatctcgcattaatctaactcaaatctattcaaattctaattcgaacctttgatatggcaggccaacgtccactgtgcctactgcgacggcgcctacgaccaggttggatttcctgacttggagctccaagtccacaatgggtggctcttcttacccttccatcgttactacctctacttctatgagaagatcttgggcaagttgattggagatgagacatttgctctccccttctggaactgggatgcaccgggtggaatgccaatgccgtccatgtacgccaaaccatcgtcgccgctctacgacgagctgagagacgccaagcaccagccgcctacgctggtggatctggactacaacttccaggatcccaccaacaccgacaagcagcagatagccagcaacctctccatcatgtaccggcaggtggtgtcgaatggcaagacggcgcagttgttcatgggtgcggcgtaccgggccggcggggagccggaccccggtgccgggtcgctagagaacgtgccgcatgggccggtccatatctggaccggtgaccggactcagcccaacacggagaacatggggaacttctactcggcggcaagggacccgatcttcttcgcccaccactcgaacgtcgaccggatgtggagcgtgtggaagaccctgggagggaagaggaaggacttcactgacccagattggctcaactcgggcttccttttctatgatgagaacaagcagctggtccgagtcaaggtcaaggactgcctcgactcggctaatcttcggtaattaaagatctgtctttctcagtttcttattctaactatactcttccagatgcctgatcccaactcctcttccaaattgggttttaggtcagcaaatgggataatgcttttagtgttagtaacctagtctttaagtttcaactctgaattaacttgtgaggttcctcagtggggcttacttgaaaaatcttaaatcctaccggatggatgtgcacgggttactccaatagaagttgaccttaccctcaaattggtcactgtaaaacatttgaatatagtgattaagccgatggtggacccaactcgtattgaaatttgatgaggaccaaagcatttattttgtgccgtttaaatattcttccaagtgatccaactgaggagccttgtgaatattgcaccatgtctgtctggcttgagaatcctgacttataaaattctcggtcaacaaaactgaataggtgaacacagctgaattacattttatgttacagttgaaagtttgattttacaacgctcctatttgatctcacataaatatctaaatttttcaaaaataaataccctctcttgtacaaatttcattcaaataagaattaattacacattcaggttgataagtcagaaaacatttattgtaatcaatttgattttgacaaagataaaatcttgggttggttaggtctgagtcagtcagtaaagatccttggaagacttttccttgtacattattatttctttgaatttggcttcctaatcccattaatctccggtccaccaaacacaggtacacctaccaagatgttgaaatcccatggctcaagtccaggccaacgcctctgaagaagaagaccgccgcgaagaaggccttaaaaggcaagaccccaactgggttccctcgagacctcgacacaatagtgaaggctacggtcaagaggccaaagaagggaaggagcaagaagcagaaggaggatgaagaagaagtgctggtgatacaaggcatagagctggagagggacgtccgggtgaagttcgacgtgttcttaaacgtggccgaggaagacgagggctcgtgcggtccgagctcgaccgagtttgtggggagcttcgtgaacgtgccccacaagcatgggaagaagacgaccaagttgcagacgtccctgaggctggggataacggaggtgttggaggacctggaggctgatgatgatgatgatgtggtggtgactctggtcccacgccaagggaaggatgtggtgtctgttggagggttgaagatagaatttagtacctga

SEQ ID NO: 2 - DNA, 1761 bp, Persea americana
atggctttcacactgaaaagcacaccatccccgctctcttcctcctcttcaaccccattccatcagcaagccaagaagaaacctcttctccttccaaaccatcgaccccaccaccttcctcaaccaatctcctgcaatagcagcaacaacagtgaaaaaaatgaaacccaaaaccatggtagaactatcgatagaagaaatgttcttctaggcttgggaggcctctatggcgctgctaccgccttctccatcgacccgaagaaggccgctgcggcaccagttctcccacctgatctctcccaatgtggagcagcagatctcccggctggtgccacccccaccaactgctgcccccccttcacccaaaagattgtggatttcaagctcccctccccctcctcccccatgcgcgttcgccctgccgctcatcttgctgataaagaatacatagccaagtatgagaaggcaattgcactcatgaaggctctcccagctgatgaccccaggaacttcactcaacaggccaacgtccactgtgcctactgcgacggcgcctacgaccaggttggatttcctgacttggagctccaagtccacaatgggtggctcttcttacccttccatcgttactacctctacttctatgagaagatcttgggcaagttgattggagatgagacatttgctctccccttctggaactgggatgcaccgggtggaatgccaatgccgtccatgtacgccaaaccatcgtcgccgctctacgacgagctgagagacgccaagcaccagccgcctacgctggtggatctggactacaacttccaggatcccaccaacaccgacaagcagcagatagccagcaacctctccatcatgtaccggcaggtggtgtcgaatggcaagacggcgcagttgttcatgggtgcggcgtaccgggccggcggggagccggaccccggtgccgggtcgctagagaacgtgccgcatgggccggtccatatctggaccggtgaccggactcagcccaacacggagaacatggggaacttctactcggcggcaagggacccgatcttcttcgcccaccactcgaacgtcgaccggatgtggagcgtgtggaagaccctgggagggaagaggaaggacttcactgacccagattggctcaactcgggcttccttttctatgatgagaacaagcagctggtccgagtcaaggtcaaggactgcctcgactcggctaatcttcggtacacctaccaagatgttgaaatcccatggctcaagtccaggccaacgcctctgaagaagaagaccgccgcgaagaaggccttaaaaggcaagaccccaactgggttccctcgagacctcgacacaatagtgaaggctacggtcaagaggccaaagaagggaaggagcaagaagcagaaggaggatgaagaagaagtgctggtgatacaaggcatagagctggagagggacgtccgggtgaagttcgacgtgttcttaaacgtggccgaggaagacgagggctcgtgcggtccgagctcgaccgagtttgtggggagcttcgtgaacgtgccccacaagcatgggaagaagacgaccaagttgcagacgtccctgaggctggggataacggaggtgttggaggacctggaggctgatgatgatgatgatgtggtggtgactctggtcccacgccaagggaaggatgtggtgtctgttggagggttgaagatagaatttagtacctga

SEQ ID NO: 3 - protein, 586 aa, Persea americana
MAFTLKSTPSPLSSSSSTPFHQQAKKKPLLLPNHRPHHLPQPISCNSSNNSEKNETQNHGRTIDRRNVLLGLGGLYGAATAFSIDPKKAAAAPVLPPDLSQCGAADLPAGATPTNCCPPFTQKIVDFKLPSPSSPMRVRPAAHLADKEYIAKYEKAIALMKALPADDPRNFTQQANVHCAYCDGAYDQVGFPDLELQVHNGWLFLPFHRYYLYFYEKILGKLIGDETFALPFWNWDAPGGMPMPSMYAKPSSPLYDELRDAKHQPPTLVDLDYNFQDPTNTDKQQIASNLSIMYRQVVSNGKTAQLFMGAAYRAGGEPDPGAGSLENVPHGPVHIWTGDRTQPNTENMGNFYSAARDPIFFAHHSNVDRMWSVWKTLGGKRKDFTDPDWLNSGFLFYDENKQLVRVKVKDCLDSANLRYTYQDVEIPWLKSRPTPLKKKTAAKKALKGKTPTGFPRDLDTIVKATVKRPKKGRSKKQKEDEEEVLVIQGIELERDVRVKFDVFLNVAEEDEGSCGPSSTEFVGSFVNVPHKHGKKTTKLQTSLRLGITEVLEDLEADDDDDVVVTLVPRQGKDVVSVGGLKIEFST

SEQ ID NO: 4 - DNA, 2368 bp, Persea americana
atggctatggcatccacatttttaagcaacaatagcttagggtccggtctaaatacgaaggccaccacctcctctgcatggcctcttcaccagcaaaggagtcaagtttctggtggtgtacgtagaaggcacagccgccgtcaatctcttctgatttcatgcaaaggtggacatgatgctgataatgctgtcccgtttattgaccgtcggaatatgcttataggcttgggagggctgtatggtgcagcaagtagcattggtttcgacgccgttgccgctccgattgccccaccggacttatccaagtgcgggccggccgatttgccggcgggtgctatcccaacaaactgctgcccacccttcaatgataagattgtggacttcaagttcccatctttgaccaaaatgagggtgcggccggcagctcacagagcggcggacgacaaagagtacatggagaagttcaccaaggccgtaaaattgatgagggagcttcctaaggacgacccaaggaacttcacgcagcaggcgaatgtgcattgcgcctactgtgatggtactgagaaactttaaacattatccacttttcaatcaatttattttttccggagatttgacacttcaatgccctttaaatatttaatctccaattttattactaagaatctgtaaatgtcatcggagtcgtttgactctgatacacaatttttctttttattttttacttttttgctctcgatcccatgatatcatgggagtttaaaaactcatgatataatgcgatccaaatgttaaaagttaaaaagctacctgtcatgggatttagagcaaaaagtaaaaaaataaaaatataccagagtcaatctgatgacatttagacacttttagttatgaaattggaaatttagtgtttaaggggcattgaaatgccaaatcctctgtatttattgatagttactacaagactacatacaatgagatgaataagaaaactgggtttcatcttaatatcaatttcatccaattataataatgaggggtcatttcttaatctaatcttgttgcaataatattaatggtcgcactctaatataactactaatcacaatttttctaacctgtattctctataggtgcatacgaccaggtgggcttccctgacctggagttgcaggtgcacaactcatggctctttttccccttccaccgctgctacctctacttcttcgaaaggatcctgggcaagctgattggggatgagtccttcgccatccccttctggaactgggacgcccctaaaggcatgataatgccccccatatacacggacccatcatcgtctctctacgacaagcttcgcgatgcggcccaccagcctcccaaggtcatcgatctcgactacaacggcgtcgatcccaccaccaccgatcgtcaacaaattatagacaatctcaccatcatgtaccggcaaatggtgtccaacgccaggacccctcagctcttcctgggctctccataccgggccggggacaatcctgacccaggagccgggtcggttgagaacgttccacatgggccggtccatgtatggaccggggaccggacacagcccaacggtgaggacatgggcaacttctactcagctgcccgcgacccaatcttctatgctcaccatgcgaacgtggaccgcatgtggaccctgtggaggcaaatggggggcacacataaggacttcacggactcggactggttggacgctgggttcctcttttatgatgaaaatgcccagctggtgagagtgaaagttagagactgccttgacattgccaagcttggatactcataccaacaagtcgaggtcccgtggcttaagtctcgccccaccaccagacgtgtggcaggtaccgcctcggtggattcagccaagaagaaggcggatgctacagacgcagcatccgtcttcccacggaagctcgactctgtgttgaaggtgatcgtgaagaggcctaaaaagtcaaggagcaagaaggagaaggaggaagaggatgagctgcttgtgatagaccagattgaggtggggcgtgatgtgcctgcaaagttcgatgttttcatcaatgtggaggaccacaagaagcatgggccggccacgagcgagttcgcgggcagctttgtgaatgtggctcataagcacaagcattcgaagaaacccacggttctcaagacgcgactgaggctggggataacggagttgctggaagacctcggagcagagcaggatgatgaagtggtggtcactttggtgccgcgctatgggaaggatgcaatcactattggagaagttcatatcgaacaccatgctgtttcttga

SEQ ID NO: 5 - DNA, 2368 bp, Persea americana; identical to SEQ ID NO: 4

SEQ ID NO: 6 - protein, 596 aa, Persea americana
MAMASTFLSNNSLGSGLNTKATTSSAWPLHQQRSQVSGGVRGRHSRRQSLLISCKGGHDADNAVPFIDRRNMLIGLGGLYGAASSIGFDAVAAPIAPPDLSKCGPADLPAGAIPTNCCPPFNDKIVDFKFPSLTKMRVRPAAHRAADDKEYMEKFTKAVKLMRELPKDDPRNFTQQANVHCAYCDGAYDQVGFPDLELQVHNSWLFFPFHRCYLYFFERILGKLIGDESFAIPFWNWDAPKGMIMPPIYTDPSSSLYDKLRDAAHQPPKVIDLDYNGVDPTTTDRQQIIDNLTIMYRQMVSNARTPQLFLGSPYRAGDNPDPGAGSVENVPHGPVHVWTGDRTQPNGEDMGNFYSAARDPIFYAHHANVDRMWTLWRQMGGTHKDFTDSDWLDAGFLFYDENAQLVRVKVRDCLDIAKLGYSYQQVEVPWLKSRPTTRRVAGTASVDSAKKKADATDAASVFPRKLDSVLKVIVKRPKKSRSKKEKEEEDELLVIDQIEVGRDVPAKFDVFINVEDHKKHGPATSEFAGSFVNVAHKHKHSKKPTVLKTRLRLGITELLEDLGAEQDDEVVVTLVPRYGKDAITIGEVHIEHHAVS

SEQ ID NO: 7 - DNA, 1990 bp, Persea americana
atggaagcaaagcattggttctctgtagtactgctgactctccttctagttgggctgtcaataaatcttctccatgattcaaactcttctttgaggtatcgaagttttttctatatttgattcttttagatcttgtttttggcttttgtctttttttttttttttttttttttttttttggaagttggggtacaagtgatctgtttatagaaatatgaaaagggtttttatataacttgcatagatttttgatggatacaatcaaacacaaattgaagttttctaaattggttttttgtaacaattgtgtagggatctaaggggattgaatgagaaaaacccagcggcctacatctcgacatcatttcaattgatccaaagcatgatcccttcaatctgggaaggtcggtcttcagatcctgaagtagcaaagcaaacaggtgggaggccaatagctccaaaccttgccacatgccacaaatcgctctccgatgcgggtcgtccagtcttttgctgcccacccaaacgcgaatccgaagagtccgtcatcgacttcaaattcccaagcccttccacacccaaacggatccgccgacccgcccacctcgtagacgacgactacctcgccaagtaccagagaggcgtgaccttgatgaagcaactcgacaccagcgaccctcgcaacttcatgcgccaggccaacatccactgcatcttctgcactggagcctacacccaagtcaactcctcccacctcctcaacatccatagatcatggttcttcttcccatggcaccgtttgatgatctacttccatgagaggatccttggaaagctgattggagatgacaccttcgcgctcccctactggaactgggacaacccacctggcatgatcatccctcactactacatgaatgggtctttcgtcgacaaggatcgggaccacgcccatctcccaccccaggttgcagacatcagcttcgactacgttgagagcggactcggccctgaggagcagatagaatcgaacctccacttcatgtaccatcagatggtgtctggtgcgaagaaggtcgagctcttcatgggctgcaagcgaaccgctggggaagagggcgagtgcgatggtcccggcacggtcgaggtcgcaccccacaacgctctccacacgtgggtgggaagcaatctccagcccgagagggagaacatgggtgccttctactcggctgctcgtgaccccgttttctacgcccaccatgccaacattgaccggctctggacggtttggagaaagcttaggggcaacgtgcccgagattgtggacccggcttggctcgactcctacttttacttccacgacgagaacgctcagctcgttcggatcaagatccgagatgctcttgacatggacaggctcggttatggctacgaagatattgacctcccatggctaaatgccaggcccaaaccctccgtcccacccaaaattgcgaaggcagtgttgaagttgagagaactaaaccagaacggattgcagtccccagccctctttagccctgacttcggacccgagggtcggatccttgacagcaccataagagccaaggtccagaggccaaagaggtacagaagcaagaaggaaaaagaggaagaagaggaggttttggttgtttatggtattgatattaaaagagatatgtatgtgaagttcgatgtctacgtgaacgtggttgatgaaaagaatacgggtcctgagggtagagagtttgcgggcaccttcgttaacgtgcgccatggcgtgacaacagtgttgaacgagggtgattcgaagatgaagatgaagagcacgctcaagttggggatttcggagctgttggaggatttggaagctgatgaggatgagagcgtttgggttacattgttgcctagaggagggactggtgtcaatactactgttgatggaataaggattgagtacatgcgatga

SEQ ID NO: 8 - DNA, 1773 bp, Persea americana
atggaagcaaagcattggttctctgtagtactgctgactctccttctagttgggctgtcaataaatcttctccatgattcaaactcttctttgagggatctaaggggattgaatgagaaaaacccagcggcctacatctcgacatcatttcaattgatccaaagcatgatcccttcaatctgggaaggtcggtcttcagatcctgaagtagcaaagcaaacaggtgggaggccaatagctccaaaccttgccacatgccacaaatcgctctccgatgcgggtcgtccagtcttttgctgcccacccaaacgcgaatccgaagagtccgtcatcgacttcaaattcccaagcccttccacacccaaacggatccgccgacccgcccacctcgtagacgacgactacctcgccaagtaccagagaggcgtgaccttgatgaagcaactcgacaccagcgaccctcgcaacttcatgcgccaggccaacatccactgcatcttctgcactggagcctacacccaagtcaactcctcccacctcctcaacatccatagatcatggttcttcttcccatggcaccgtttgatgatctacttccatgagaggatccttggaaagctgattggagatgacaccttcgcgctcccctactggaactgggacaacccacctggcatgatcatccctcactactacatgaatgggtctttcgtcgacaaggatcgggaccacgcccatctcccaccccaggttgcagacatcagcttcgactacgttgagagcggactcggccctgaggagcagatagaatcgaacctccacttcatgtaccatcagatggtgtctggtgcgaagaaggtcgagctcttcatgggctgcaagcgaaccgctggggaagagggcgagtgcgatggtcccggcacggtcgaggtcgcaccccacaacgctctccacacgtgggtgggaagcaatctccagcccgagagggagaacatgggtgccttctactcggctgctcgtgaccccgttttctacgcccaccatgccaacattgaccggctctggacggtttggagaaagcttaggggcaacgtgcccgagattgtggacccggcttggctcgactcctacttttacttccacgacgagaacgctcagctcgttcggatcaagatccgagatgctcttgacatggacaggctcggttatggctacgaagatattgacctcccatggctaaatgccaggcccaaaccctccgtcccacccaaaattgcgaaggcagtgttgaagttgagagaactaaaccagaacggattgcagtccccagccctctttagccctgacttcggacccgagggtcggatccttgacagcaccataagagccaaggtccagaggccaaagaggtacagaagcaagaaggaaaaagaggaagaagaggaggttttggttgtttatggtattgatattaaaagagatatgtatgtgaagttcgatgtctacgtgaacgtggttgatgaaaagaatacgggtcctgagggtagagagtttgcgggcaccttcgttaacgtgcgccatggcgtgacaacagtgttgaacgagggtgattcgaagatgaagatgaagagcacgctcaagttggggatttcggagctgttggaggatttggaagctgatgaggatgagagcgtttgggttacattgttgcctagaggagggactggtgtcaatactactgttgatggaataaggattgagtacatgcgatga

SEQ ID NO: 9 - protein, 590 aa, Persea americana
MEAKHWFSVVLLTLLLVGLSINLLHDSNSSLRDLRGLNEKNPAAYISTSFQLIQSMIPSIWEGRSSDPEVAKQTGGRPIAPNLATCHKSLSDAGRPVFCCPPKRESEESVIDFKFPSPSTPKRIRRPAHLVDDDYLAKYQRGVTLMKQLDTSDPRNFMRQANIHCIFCTGAYTQVNSSHLLNIHRSWFFFPWHRLMIYFHERILGKLIGDDTFALPYWNWDNPPGMIIPHYYMNGSFVDKDRDHAHLPPQVADISFDYVESGLGPEEQIESNLHFMYHQMVSGAKKVELFMGCKRTAGEEGECDGPGTVEVAPHNALHTWVGSNLQPERENMGAFYSAARDPVFYAHHANIDRLWTVWRKLRGNVPEIVDPAWLDSYFYFHDENAQLVRIKIRDALDMDRLGYGYEDIDLPWLNARPKPSVPPKIAKAVLKLRELNQNGLQSPALFSPDFGPEGRILDSTIRAKVQRPKRYRSKKEKEEEEEVLVVYGIDIKRDMYVKFDVYVNVVDEKNTGPEGREFAGTFVNVRHGVTTVLNEGDSKMKMKSTLKLGISELLEDLEADEDESVWVTLLPRGGTGVNTTVDGIRIEYMR

SEQ ID NO: 10 - DNA, 2974 bp, Persea americana
atggaaagtggcgtctgctacagaggaggaattcctgcctttgaagctttgccctctggggtgaagtccaaaggagttcttactgtttccgcctcagccatccgatctcgctacgttgctagtatctccatcgttcgttctcaggtttgagcttctgatctgtgtaatctagtctctttttaataaaatccatttgaatgttttgttgggtttttagtgattgttaggatctggtgagtgatggggtgggatttgttgtttgtatttcttttgatcgagttacgagaaatagaaccgtttgttgataaacaatgcaattttaagatcttcaacatggggttcggctggaattggaagatgaagacagatgttataaccaacggtcttgatttagtagtagtaggacacacaagtctgtttctgggggtgtttgattcgtgtcttttttgggctttgggccgtctatattgggatccaagatataaagggaccagatcgattggtggggttgcccagtgttttgtgggagcacatggtagattgtgcgatggcactgaaagcttgagacacaaccactctagtgatgttgatgttccttgaatggaaaagatgagacatttctaatgtttttatttctatttcaaagccatgaaatagaagaaatttatgttcttttttaagttggaatagacccccacaacccctgcatagcaggatgatgataacaaagccatcttcctttgggcacacttagtgggccccacaaactctcccattttcatagaattggttgtgggttcctgcttctaaatctgtagagaggatccctgtccttttctaaaagatcctgaaatgaatgtacaatttttttttttgacacattggttgtctagtattggtggcttttgatcccaaaggagaaacatttcaccatcgtcttcattttagtcttatcctatgaaggggagtcttattttagtctttaatttgcctttttttcataatgtagggtatggtcaacttctgtggaagccggcaatttggagagaaggttaagtgtggcactctgagaagcccggctacatttgtcacagtcgccagtgcagagtctagtaagtgtgattttaggagtgtggcgacacccctcgaaccacaatcatcggctggaaagtttttgagtgatatattgaagaatcaccctcacattttccatgtggctgctgcggagcagctggagcatttggctgcagatagggatgatgctgtcgctcggcgggagcagagcttgggttcacctgaatcatgccttcataggttagtattattaactcttgatttcttcaatcatcaaatgtggatttaaactgacaaatgttctttttggttcccgtgatcaactgctctttctcgtttgatcatgattttctgtcactggtagtattgcctattaaagggtgcaattttttaaacatgttatgttcattgtttcaattgaccggactgcagtgtctaaatagtttttcatctgctggtccatcatcttctgttacaattattaagatgtgcattacatcctatatttatgcttgcatgtatagacctgtgttacctataactgttgaagtatgaaaaatgttagtgccaaatgcagtagacacattgctcaagatatgtgaaagcataacaactattaatgatttcagtcactatttgatatggtatgttttgccaatttctacgatttccaacctagatcttgggcaatcaccaagattggaacaccctaggcagcatctcaaccatattttatggattgtacacattgttgcagaagttacaaattaaagctcttgaaatgattgtgttgttttggatgcgtgattaaggaatggtatgattcacataaccaattctcatgcaagagtgtgtgtgcatgtggaacatatggctggtggttcaaaccataggttctgttgggcttgagttccttcaggcactgaattttcaattggtcaggcgaggcctattcaaattgacagttctagagcctcgtccattgacagccctacccagtataaataatctttaactcaaaattgcctagatcggacaattgtatttcacgtgtgtcaccagttaccctgttaacaaatgtgtgatattggatgccaggagaattgcagaaatgaaggagggtgagtgccaaattgcgatcgaagaggtcatgtacatgctagttgttcgaaagttctctgagattgatgtcccaatggttccaagattatccaaatgcatcaacaatgggagactagatatatggacgaccaaggacagagaactagagtccattcatagcttagatgttctggaattgattagggaacatctctcaaccattctaggctggagaggaaaatccgatgtcacagataactggacaacaactcagatttgcaggctgcagcttggccgaatttatgctgcttccatcatgtatgggtactttctgaaatctgcctgcctgcggcaccgcctggagctaaatctcagtctgactcatgtagacctccctcctggacatgagattgaacacccacttgcagaaaggcggccttgttcacttggaaatcttgctgttgctggctgtccaaatgatacaatatcttcattgtatcaaggatcaggaagggacaggagaactgagaagctgaagaggtatttgatgggatttgatcccgagactttacagagatgtgcaaagttgaaatcgcaggaagcagtaaaccttattgagaagcacagttgggctctgtttggagaggacaatgagtcaggttctatagacagcgacgaggcgattgctgtcacattttcaagcctgaagaggttggttttggaggctgttgcatttgggtctttcctttgggatgtggaaaggtatgttggttccttatacaggttaaagatgacctaa

SEQ ID NO: 11 - DNA, 1251 bp, Persea americana
atggaaagtggcgtctgctacagaggaggaattcctgcctttgaagctttgccctctggggtgaagtccaaaggagttcttactgtttccgcctcagccatccgatctcgctacgttgctagtatctccatcgttcgttctcagggtatggtcaacttctgtggaagccggcaatttggagagaaggttaagtgtggcactctgagaagcccggctacatttgtcacagtcgccagtgcagagtctagtaagtgtgattttaggagtgtggcgacacccctcgaaccacaatcatcggctggaaagtttttgagtgatatattgaagaatcaccctcacattttccatgtggctgctgcggagcagctggagcatttggctgcagatagggatgatgctgtcgctcggcgggagcagagcttgggttcacctgaatcatgccttcataggagaattgcagaaatgaaggagggtgagtgccaaattgcgatcgaagaggtcatgtacatgctagttgttcgaaagttctctgagattgatgtcccaatggttccaagattatccaaatgcatcaacaatgggagactagatatatggacgaccaaggacagagaactagagtccattcatagcttagatgttctggaattgattagggaacatctctcaaccattctaggctggagaggaaaatccgatgtcacagataactggacaacaactcagatttgcaggctgcagcttggccgaatttatgctgcttccatcatgtatgggtactttctgaaatctgcctgcctgcggcaccgcctggagctaaatctcagtctgactcatgtagacctccctcctggacatgagattgaacacccacttgcagaaaggcggccttgttcacttggaaatcttgctgttgctggctgtccaaatgatacaatatcttcattgtatcaaggatcaggaagggacaggagaactgagaagctgaagaggtatttgatgggatttgatcccgagactttacagagatgtgcaaagttgaaatcgcaggaagcagtaaaccttattgagaagcacagttgggctctgtttggagaggacaatgagtcaggttctatagacagcgacgaggcgattgctgtcacattttcaagcctgaagaggttggttttggaggctgttgcatttgggtctttcctttgggatgtggaaaggtatgttggttccttatacaggttaaagatgacctaa

SEQ ID NO: 12 - protein, 416 aa, Persea americana
MESGVCYRGGIPAFEALPSGVKSKGVLTVSASAIRSRYVASISIVRSQGMVNFCGSRQFGEKVKCGTLRSPATFVTVASAESSKCDFRSVATPLEPQSSAGKFLSDILKNHPHIFHVAAAEQLEHLAADRDDAVARREQSLGSPESCLHRRIAEMKEGECQIAIEEVMYMLVVRKFSEIDVPMVPRLSKCINNGRLDIWTTKDRELESIHSLDVLELIREHLSTILGWRGKSDVTDNWTTTQICRLQLGRIYAASIMYGYFLKSACLRHRLELNLSLTHVDLPPGHEIEHPLAERRPCSLGNLAVAGCPNDTISSLYQGSGRDRRTEKLKRYLMGFDPETLQRCAKLKSQEAVNLIEKHSWALFGEDNESGSIDSDEAIAVTFSSLKRLVLEAVAFGSFLWDVERYVGSLYRLKMT

SEQ ID NO: 13 - DNA, 1327 bp, Persea americana
atggaaagtggcgtttgctgtggtagaatccctgccgttgaagctttgcccgctgtggtgaagtcagaaggcggttctaggattttctcagctgtgattgggattggggccctcagatctcacaatttcgctcgcatctccttcgcttctcaggtctcttcttcttcacccaccctcttatcacttctcaggcctttgcatatttggtggggctgtgttacaagatctggtccgaaaagaattcatagccaaaggttttgattcaccaagtggctgatgagtttgtagccaaaggttttgattcaccaagtgcctgatgagttcttagccaaaggttttgattcaccaactacctgaatcttgggcatgcttaaaaacgtggggtgcccaccataatgaaaggccagatctctggtggggtactcccagattttgttgggagcacatgaacaatgaacccttacccacccttatatctgacccagatgttctttgacaggaatatgaatgagttattttctaaatctatggatgcaaagagaacatgttaggaacattgatattatcatcatcattatcaccaataccccaagaccccttaccacctaggaattagctcccaccaaacttatttgattattttaagcccttgggagttgatcatgtaaatttggcacccatctgattatgaactgactagaattttgacagccttagagccctcaaaagtttacctcttttgttcaaatggacattgattgttgtatagaggtgggtcatgagggcatgaggagacctttcatccctattgtgggagccacaggccaggctgttccatgccaagtgaaagcctactgttcctgaaaatgactggtggggattacagtatgtgtatatattttaattcttacacgatcagctaaggagaacttgactaggtcttgtgacttttggttaggaaggaggccgttggatatctctaattgtttcttctttattactaattatatagggtttggtcaacttctgtggaagccaatgttttggtgggaaggttgggtgtggtagttggaggagtccatttgttacctttgccagcgcggactatagtaaatgctattctaaaagtgtggaaacgccccttgagccaaggtcatcagctggaaaattcctgagtggtatattgaagaaccatccacacattttcaatgtggctgctgcagaacaactagaggaattggttgcagagaggaatggtgcattcgctcgacgtgagcaaagcttgggttcaactgaattatgccttcatgggttagttgcccgataa

SEQ ID NO: 14 - DNA, 468 bp, Persea americana
atggaaagtggcgtttgctgtggtagaatccctgccgttgaagctttgcccgctgtggtgaagtcagaaggcggttctaggattttctcagctgtgattgggattggggccctcagatctcacaatttcgctcgcatctccttcgcttctcagggtttggtcaacttctgtggaagccaatgttttggtgggaaggttgggtgtggtagttggaggagtccatttgttacctttgccagcgcggactatagtaaatgctattctaaaagtgtggaaacgccccttgagccaaggtcatcagctggaaaattcctgagtggtatattgaagaaccatccacacattttcaatgtggctgctgcagaacaactagaggaattggttgcagagaggaatggtgcattcgctcgacgtgagcaaagcttgggttcaactgaattatgccttcatgggttagttgcccgataa

SEQ ID NO: 15 - protein, 155 aa, Persea americana
MESGVCCGRIPAVEALPAVVKSEGGSRIFSAVIGIGALRSHNFARISFASQGLVNFCGSQCFGGKVGCGSWRSPFVTFASADYSKCYSKSVETPLEPRSSAGKFLSGILKNHPHIFNVAAAEQLEELVAERNGAFARREQSLGSTELCLHGLVAR

SEQ ID NO: 16 - DNA, 2391 bp, Persea americana
ctccaacccatacattatgtggaccctacttctaattaattaagatatacacctcatcttcttcatccttcacatgaatcaccgtcaatggcatctttcctaaatccccaattcctcacccacaccatctcctccaacagacccttcctccatcgctctctcatctgcgctcacaaacccgattcccaatcctctccaacccacaggcgtcggatcctaatcggattaggaggatcgctcctcctatctgctgctgcttcttcttctttattctcccgacccagaaccgatccactccatccaaccgtccaatctcaacatcccaactcgtggcccaccatcttccaaatggacgcggcagaagcttccacgatcgacggagaattcccatgcgtgttggactcggtggtcaaagccacggtcaagaggccgaagaaggcgaagagcggggaggaagaggtgctggtggtggacgggatcgaggtgtataacaacgtgcccgtgaagttcgacgtgctgatcaacgtggcggattggcgcacgtgcgggcccgggtccagcgagttcgcggggagcttcgtgcacgtgccgaggaagccgtgggacccggaggggaaggtgaagacgcgccttaggctggggataacggacctgctggaacagattggagctgatagggatgatgagttcacagtcacttttgtgcccagggctggaaattatgtcagagttggaggggttaggatcgaatacagttcttgattgggtcagttctatctatgctcagtatgaaaacactttatgttttttttccaaatcttgcaccatgtatgggccccacaagaaatagtgctcacctgaggtggggccggtctgcgtgaatagactttgctgtatgtgtgtttgaagatgatggtcttctttcttggaatgtctgtatattgtatctattgacagtctttaacaattcctcagtgttatttgtttttaattttgatgacatcttgatctggcccaattggcattagcatatccttttaggtggaagttagagatggtcgaatgaaaagagcctctgtcaaaatctccacataagtagtgaaggatctgattgaaccacagtaagatgaggttgttgtggatcaaacataaattggctcattatttgaacaacatgagcaatgtctggacgggtgatgatgaagtaaatgaggcttctaagtgacatatagaccatcatcagcgatatggtagttagtttgttaggcgagaaagtataaccaaaccctccacataatttcttcaatcaatgtgaggcccatgagcagtcatcttaatctccaggcctagaaaatatgttagatgtctcaaatctttaatgtggaaatgtttggcgaggtgagcttttagattagtaattccagtagaataagtaccagtgattaccgtgtcatccacatttaggagaagaattggaataccttaccttttgaagttcttagtgagaaaagaatggttatgtgcactctgatggaaagcaacttttataacagcagtttggaatttctcaaactagacacactggacttgcttaaggccatcagacttattcagatggcctgaggggcaataatgccaaggaggaggagacttttgtacaacctcgtgaaggtccccaatcctgagtgtcttcccggttgcctgatcctgcagcacaactagaagagaaagcaacttttaaaaatcgtccacaagttgaccaacagagataagattagcagataaatcgtggacataacaggtatcaagagatgcaaaggttggctgtgacaattggataacacaatcgtaccagtactagcgatgggcaaatggttaccattggcatttatagtatgcccttttctagtgtagggttccatatgatgaaatgtagatttgactgaagtcatttggttgggtaatcctgaatctgtgtagcatgatgacatgggatgaaatgtaggctagcagggtaagaaagagttactggatattcccatagcagagaatgcactttgtgtgcatagggcaatagagctttcaatgagttctttaatctgctctggagtcagggaaggagattcagaggcagagacgggcacaacatcagtactagcttgggctttcaacgttgttgggcctggaccattacttggaggggtagatgtgtcttgcatactagacgttaggagtggggaatcttggctgagagaggacagggattgcaagctatgatgcacagataccacaaaatgggcagcgtatcgtgtcgtatcgtatccgatgcttcactttttatgtgtaaaatctatgtccatgtcatgtctacggcgtgtccttgccgtatcc

SEQ ID NO: 17 - DNA, 675 bp, Persea americana
atggcatctttcctaaatccccaattcctcacccacaccatctcctccaacagacccttcctccatcgctctctcatctgcgctcacaaacccgattcccaatcctctccaacccacaggcgtcggatcctaatcggattaggaggatcgctcctcctatctgctgctgcttcttcttctttattctcccgacccagaaccgatccactccatccaaccgtccaatctcaacatcccaactcgtggcccaccatcttccaaatggacgcggcagaagcttccacgatcgacggagaattcccatgcgtgttggactcggtggtcaaagccacggtcaagaggccgaagaaggcgaagagcggggaggaagaggtgctggtggtggacgggatcgaggtgtataacaacgtgcccgtgaagttcgacgtgctgatcaacgtggcggattggcgcacgtgcgggcccgggtccagcgagttcgcggggagcttcgtgcacgtgccgaggaagccgtgggacccggaggggaaggtgaagacgcgccttaggctggggataacggacctgctggaacagattggagctgatagggatgatgagttcacagtcacttttgtgcccagggctggaaattatgtcagagttggaggggttaggatcgaatacagttcttga

SEQ ID NO: 18 - protein, 224 aa, Persea americana
MASFLNPQFLTPTISSNRPFLHRSIICAHKPDSQSSPTHRRRILIGLGGSLLLSAAASSSLFSRPRTDPLHPTVQSQHPNSWPTIFQMDAAEASTIDGEFPCVLDSVVKATVKRPKKAKSGEEEVLVVDGIEVFNNVPVKFDVLINVADWRTCGPGSSEFAGSFVHVPRKPWDPEGKVKTRLRLGITDLLEQIGADRDDEFTVTFVPRAGNYVRVGGVRIEYSS

SEQ ID NO: 19 - DNA, 2844 bp, Persea americana
atggcatctctttccttcctttccaccacccaaaccgctcccctccaccaccccagaaagccccatcaaccatctccaacctgcattgcccgcacaaggcgtttccacacttcttgcaatagtaccagcagtaacaacaccactcctaatactactaatactactgatacttccaccaagagaggagtacccggatccggatccggatccggatctcctctccggttggacaggcgcaatgttctcttaggcctaggaggcctctatggtgcaaccagcctcccaggccgggagaaaattgcgcttggagcgccaatatctccaccagacctctccaaatgccaccttgccgacggcggcaccggcgtcggcaatgttcaatgctgccctccttactctagtgacactgtacctattgactatcagttcccggcatcatcaaagccgctgcggattcgccgcccagctcatttgctagagaaggaggagattgagaagtataaggaggcaatagccaagatgcgggaactgactaccacggatccgagtgacccgagagggtggatgcagcaggccaatgtccattgccagtactgcaatggcgcctatgaccaggttggctacgacaatgtccggctgcaggtgcacttcagctggctgttcctgccatggcaccgctggtacctccatttctatgagagaattctggggaacctcatcggcgacgatagcttcgcgctcccttactggaattgggacaccccacttgggatgtacgcaccaagtatattcgtcgacaccacctcgtcgctctacgatgagaatcgtaatcttagccactacccgccggcggtgctcgactacaagtatgcctatggtgacgccgtcccatccacagaggaagcagtgcaagaggtgagctttgacactatcttaatttggagcttttgaaaacaaaaatcaatagacatggaagtatagataatccatgcctcataagaagtatagatgatccatgtctatagagttttctttttctaaagaagtgatgtgtgctaacatggtctcttgtgaggttaggtgtaggactaagacattcttctttaaaaaaaatgtggttaattaaggagtgtgagtgctttatgtgaccctttaatattaactatgacttttggttaaatgcatgtgtaaaaagtattagaacaccttttcatttcttcgtcttttggtgttgtgtatcatggggacgactttttgtacaacaagtgggatattgtaatgagtgaaaaatctaatgattttccatttagcatacttttctcttatttgatgccacactttctcatatggaaatgagaaaaaatgaactaataggccttggtatagaaaagatttcaatttcctatcaaagaagtagataaacagcaggttttctgtgatttttttttaaatttttttgctacaggttgttaatcaaaacctattggagctgagcaagacgtacaaggagagcttgaccctgcccgagctgttcatgggggacccaataagggccggggaggcaactgagacagacacggaagcctcatcaggaaggcttgagatcatacacaatggggtgcaccagtggaccgggccagacacggtcccttacatggacatgggcaacttctactcggcgggccgagaccctatcttctactgccaccactcgaacgtggatcggatgtggcagatttacaggtccatgaggggcaacaagaccgagttcaaggatgatgactggctcaattcctccttcctctttgtagatgagaacaaacaactagtgaaagtgaaggtagcactgcaatgacattatattaaagcactctctacatcatagttttggttttgcttttttctctttccacacccaagccatacattgcatctgtccttttttggtttttctagtggatcactgaagaagatgcctagtctatgccaccaagcccactatagaagacagagacctcacccacccaagcaatagctggtcaagaaagttcctctttttaattcataaagaaaaaaaacagatggtcccaacgaatctaaaattataattaaaatacaagtggatgattaattttcatgatattcgcctaggatccttttgcacatggacagtgatttcagactttttatacttttcaatagaagtagttagatgaacaaattaaacaattaaggatgtataatttgttgttgatgatatttgaacatttgtgggtggcacaggtgcaggactgcttcaaccccctcaaactaaaatactcctacgaagaagtggagctaccatgggccgaggtgggtatccgcaagaaactgaccaaggtcacggccaaggccaagacactgtccttgatcaaagtaagcgagttcgggtccgatccgaagacccttgacaaggccaccatccgggtcctggtgacccggcccaagaagtcaagatccaagaccgagaaggagggtgctgtggaggttcttatcatcaagggcatccaggcacccatcttcgagccatctaggttcgacgtctacatcactaccccctatgagggtgacctagtagccccgagccttggtgagtttgcaggcagcttcacaaagctgccccaccatggcagtgggaaggatacaggtgcgaccaagaccaagaagtctaagctcaagcttggtatcaacaacttgctggaggatattgatgctgagggggctgagaagctggtggtgtccttggtcccacgtttggggcaggttactgttggtggtgtaagcattgacctcctgaacacttga

SEQ ID NO: 20 - DNA, 1851 bp, Persea americana
atggcatctctttccttcctttccaccacccaaaccgctcccctccaccaccccagaaagccccatcaaccatctccaacctgcattgcccgcacaaggcgtttccacacttcttgcaatagtaccagcagtaacaacaccactcctaatactactaatactactgatacttccaccaagagaggagtacccggatccggatccggatccggatctcctctccggttggacaggcgcaatgttctcttaggcctaggaggcctctatggtgcaaccagcctcccaggccgggagaaaattgcgcttggagcgccaatatctccaccagacctctccaaatgccaccttgccgacggcggcaccggcgtcggcaatgttcaatgctgccctccttactctagtgacactgtacctattgactatcagttcccggcatcatcaaagccgctgcggattcgccgcccagctcatttgctagagaaggaggagattgagaagtataaggaggcaatagccaagatgcgggaactgactaccacggatccgagtgacccgagagggtggatgcagcaggccaatgtccattgccagtactgcaatggcgcctatgaccaggttggctacgacaatgtccggctgcaggtgcacttcagctggctgttcctgccatggcaccgctggtacctccatttctatgagagaattctggggaacctcatcggcgacgatagcttcgcgctcccttactggaattgggacaccccacttgggatgtacgcaccaagtatattcgtcgacaccacctcgtcgctctacgatgagaatcgtaatcttagccactacccgccggcggtgctcgactacaagtatgcctatggtgacgccgtcccatccacagaggaagcagtgcaagaggttgttaatcaaaacctattggagctgagcaagacgtacaaggagagcttgaccctgcccgagctgttcatgggggacccaataagggccggggaggcaactgagacagacacggaagcctcatcaggaaggcttgagatcatacacaatggggtgcaccagtggaccgggccagacacggtcccttacatggacatgggcaacttctactcggcgggccgagaccctatcttctactgccaccactcgaacgtggatcggatgtggcagatttacaggtccatgaggggcaacaagaccgagttcaaggatgatgactggctcaattcctccttcctctttgtagatgagaacaaacaactagtgaaagtgaaggtgcaggactgcttcaaccccctcaaactaaaatactcctacgaagaagtggagctaccatgggccgaggtgggtatccgcaagaaactgaccaaggtcacggccaaggccaagacactgtccttgatcaaagtaagcgagttcgggtccgatccgaagacccttgacaaggccaccatccgggtcctggtgacccggcccaagaagtcaagatccaagaccgagaaggagggtgctgtggaggttcttatcatcaagggcatccaggcacccatcttcgagccatctaggttcgacgtctacatcactaccccctatgagggtgacctagtagccccgagccttggtgagtttgcaggcagcttcacaaagctgccccaccatggcagtgggaaggatacaggtgcgaccaagaccaagaagtctaagctcaagcttggtatcaacaacttgctggaggatattgatgctgagggggctgagaagctggtggtgtccttggtcccacgtttggggcaggttactgttggtggtgtaagcattgacctcctgaacacttga

SEQ ID NO: 21 - protein, 616 aa, Persea americana
MASLSFLSTTQTAPLHHPRKPHQPSPTCIARTRRFHTSCNSTSSNNTTPNTTNTTDTSTKRGVPGSGSGSGSPLRLDRRNVLLGLGGLYGATSLPGREKIALGAPISPPDLSKCHLADGGTGVGNVQCCPPYSSDTVPIDYQFPASSKPLRIRRPAHLLEKEEIEKYKEAIAKMRELTTTDPSDPRGWMQQANVHCQYCNGAYDQVGYDNVRLQVHFSWLFLPWHRWYLHFYERILGNLIGDDSFALPYWNWDTPLGMYAPSIFVDTTSSLYDENRNLSHYPPAVLDYKYAYGDAVPSTEEAVQEVVNQNLLELSKTYKESLTLPELFMGDPIRAGEATETDTEASSGRLEIIHNGVHQWTGPDTVPYMDMGNFYSAGRDPIFYCHHSNVDRMWQIYRSMRGNKTEFKDDDWLNSSFLFVDENKQLVKVKVQDCFNPLKLKYSYEEVELPWAEVGIRKKLTKVTAKAKTLSLIKVSEFGSDPKTLDKATIRVLVTRPKKSRSKTEKEGAVEVLIIKGIQAPIFEPSRFDVYITTPYEGDLVAPSLGEFAGSFTKLPHHGSGKDTGATKTKKSKLKLGINNLLEDIDAEGAEKLVVSLVPRLGQVTVGGVSIDLLNT

SEQ ID NO: 22 - DNA, 9984 bp, Persea americana
atgtctcttcatcatctaacgaccaccaccccactttcaacttcatccccccaccaaaaaactcaattccaaaagcttgacaaaaagcacttatctgtttatacaagcagaaggacaacagggtggccaagtagtattagaagtagtagtactaacagcaatggtgatgagactattgctggtgaagagcaatctgcttcttcgaaacgggtcgaccggcgagacgtcctactcggcctgggagggctgtatggtgcagctggtctcgccggccaggccctggcgtcgccggtgaccatccccgaccggaatgcctgcggcattgcaacgtccccagtactccccgggccaatttattgctgcccaccagagaaagtaaggactgctcctatcgtccagtggcagtcttccaacaagggtccactccgggtcaggaaaccggcccaggagatgaacaaggacgaggtggccaggttcaaagcggcagtgcaggctatgaaagatctggatccggaggacccatggcacttcgaccagcaggcgaaaatccactgcgcctactgtaacggggcttacaagcaggtgggcttcgacgtccctcttcaggtccacttcagctggctcttcctcccttggcaccgctggtacctctacttctttgagaggatacttggaaagctgatccaggacgagagctttgctctcccattctggaactatgacaggccggaggggatgtatatgccgagcatctacgtcgacccatcctcgtccctctacaactccaaaaggaacccgaaacatctggaatcgctcctggatttcaattacagctacgatgcagatggcttaacggggacggagaaggaggtcattcaggcaaacctagtggaactgcggaccatgtacgacagtggcattcccacgccagagctgttcatgggtgacccggtttccgcaggtgagctgacagactgatattgagacccgattttaattaaaactttacaacggattaagtaatttcaacttgcatcatctatgcggatcaacgtgggttgacttgcataagttgcactcaatgacggtccatttggtttaattcaatctccctgtattacacgggtcaaaaaggtttttatccccatggcatacagattgactgtgcatcaatccgaatggattatacacacgttaaaattgtgtggtttggatgaaaatggtagcatgacttccaaatttggtatgtaggtgagacccaactggatcttttcatatgggaccttcagtgcgcttactgtgtgaaatctttgcacccgaacaaattcatttgtatattaaaactatatttaggttaagttatagggaaaattttaaaacccaactataattcaagccttcgtttcaaatatgttggagtctgtctacaaaaattctgtttatttataccaataatcgacagtcaaatatctccgtaagttgcacttttttacctctattgctaccacttccataaacatataaccacagagttatttaatatcttttcattggcgacctggtttgtgcaggagaggagaccgcggaggacaactcgtccggctcgctcgagaggttccacaacacggtgcacatgtgggttgggagacacaagaatgatgcgaccgagccctacgtggacatgggtgacttctccaccgcggctaaggacatgctcttctacgcgcaccattccaatgtggaccgcttgtgggaaatctacaggacgcgccgagggaagaagttggagttcaaaagcaacgactggctcaatgccgagttcatcttcttcgacgagaatagacaggtggtcaaagtaaacgtgaacgattctctaagtacactcgatctggggtacacctataaggatagtgttcccactccatggttggaacctgcacgtcctaaaagaccagcagctaagcctaggtccgggtccttctctatggtccctgtgaccgagtttgggactgaacccagagctctcgtggacgcgcctgtccgggtcttggtctccagaccgaagactagccgtagccaggatgagaaggaggacgagaacgaagtcctcgttgtcgatggaattgaagtggtagaggaaggggctgtcaggtttgatgtctttctcacctccccgtttgggaactttgcaggacccgactatgggttgcctgcagggagcttcgtgaagctgccccataaacataaggcagggagcaagcagaggaaggcgaagctgaagctggggattacgaagctgttggaggacctcaaagctgataacgcgcagaagctggttgtgacctttgtgccccggactgggagtgtgaacattggaggagtacatgtggaactgttcaagactgataattagatgtctggtttgggtctcggtcgttttagtatggatggtcagttgttgttgccgtcgcttgctttgctttgagttaataagatgttgaatctaatttcaacccaatcgagtatatggtatgcatgtgctgtacgtatctttaatcaagtcttatcaatgctgtttcttgtcagcttccatcaagtagttgattccttttctttctttttcattttgaaaagtagaacacattgcagacaaaataaaattagaggccctctttggatcttaggactggtgatggtatagcacaaaacaacttgcagttgttcttggtggcctattcaaattcatgaaccctcatttatgcaaaaacttaaatacagtcaggtttaagaggactccaaaggaatatggaaacagaaacaattacactaataaaaaaagggtatttagaattgagaaatattcaagtgagatctgccgtgggaactaagggtcgaagtgggacccaccgatgaggctatcaccgaatctaaattttgcaatagatgggggtccccctcattgtcactcacccttaggtttggcactcaagcaaatttactattcaaacttgatatttgaggtggccatactggaaaattttcatatcccgtgatcttaatcttgttgagagagaaggaaagaaagaaactataaatacaacggaaaaccaatttattgcattctactaaactgcattgcatcaaccaatataaatagataagccccaagcagccctactaaataaagtccgaacaaagataggaaacacaagcacactaacaacagtatattcctagaaatattcctaaagtacatatatagcttcactggcccaatccttagtgtgcctgttgaatcaacaatgacttcaacaatctgcatatggaacccacacagggtcccacagaaaatttacatgaaacttcaaaagtgggtctaagtgggccagaagggactaagtgggtaaataagggtcatattaaatatgctgacttaaataaataagattattgatgataaatgaagttcatgatcgtgacaattaccctattaaacaaataaactaaatcatgaggatgaattaatttgaacaggctgaactatgtgatgatggatttagttaaaatgaactaagtgatgatgaattaaattaaaatggcctaagtgatgatgaattaaattaagataaaagaaaaacggaagaatctcacgggtattttaataaaaatcagggtcgaataattggagatttgcccgttggtttattggcttgagtccaaagtgggtatagtctgggctgatgctaatgactgggctaggtataggtccgactgggttgcgaattgagtgggtatggtttgagtaggatgttggtttgatgggctcgggttggtggaccagatttgagtggatcgaggcaggtgaggctcaggttgagtgggtacgtccagtggctggactcgagatatgtgctggtgggtttggtatcagacttgactgggcccagtcaagtgggttgtatataggtaaaagggccagtgagagtggtgagttggttcggaagggttatgagaaggataaggttggctcggttgcagttgggaaatgggcgtaggttcagttgtttgagaaagataatgaaggaagggggagatggagaaggtgtttggtgcaaatgggtagtgggtgcagagtttttcgactaggtggctgagatgagtgaggatgatcatgagttaccgaactaaataaccaagttcaaatttaagatgaataaacgtaaacttcaaactaatgcgatattaatgtgatgcaaacatgacaaagatgtaggccacgatcaaaatcaataaacttggatgccaactgcatgcaagttgaccgatccaccagatgtatgtaaaacgcatggtgaaaatccggataaactaccgtgtaaaacaagttggacaatcaaaaggatagaattatatgtcaagaaaagattacccggtttgcatatagttttgtgtgtaaggataaggtgtagaaatatttctcacgaagagatcctccccacttatgcttttaccgcatgttacaatgtcaagttcgttgtgtttggttggaggtgcaacagtgtttggctggatggattcggggtgatgttggggttgtagctgtgttggagttgagtgaggattgtggatggagaaggtgatgaaggtttttgtctctatgagaggtgaggttagtggtgtgttgttgtgattgtggttcgttgatgagtggagatgagaaaggtcgcgagggggttgtagaaagagagtgagaagagagagtaaataatggagagaggttggccaagaaaaagagggagtcagccatgggagaaaagaaagaagaaaagtgatggctcatgtgggcgtttctttttataagaacagggcaacagggcgcgcccgagaaagagagacaaatgggctgagcgcactcaaatgggttgttggattttgtgggctactcaacattgcggacccaaaaattttataccagtttttttggggtgggggaaatccttatatctggctacgccttgcacaactatttcggccctttctacgaaaattttaagatttagtggcccaatcatcatcttaaccccatggaggattgtatttatcaatctattggtgggcctgaccttctcgagagtccaacacatactaatttttaaagtctgtatcttgagatctgaccgtttggttttttgagatcttcatatatatattattggagatttctcaactgagtgggcatgagagtatatgcatatgacccatttgaagacaagaaaaaggacttgagttttcacagtgaccatggttagaaattgatatcttcccactggatttacgaggccaatataccactggaaaactcacaatgaaaagatgatcattgaactgagattcgacctaaaaatgagacctagggctatccaactgaaaatgaaatctagatgatgtaattatcccttagatatgcatgatgatgaattggccttaggattttaaactattcatctaaaatatttgaatggggcaaaatcgagctgtcactattatggatatcagaattgtgtatggggtgctaaaatgaggtgccataacaggaatcattctttggggtgtgtgtgtaatattgaggtaagaacaccacccaaccatgtgttggccaaatcggagctttgggtaggcttttaatcgagagtccgtatccctgagtggtccctcacacgcttcttgtgagcaggatgtgtggaactctactttgtgacccaccaattgatgatccaaacagctcatattgaatgtcactcacttcaatgatatatgaatgcatatagttaataatatgtaagattgtgataaaaatctattacattaaaatttgaggatcagctgtggaggtatgtgttttgtatggctccaagcatcccacccacgaggagcatgtggggagacggttcataggagtcggccacggttaccgaaggtctctctgtttttaattgatttataggaacttctcgcacactaagaaaagattctccccacccagtcacccaccttctaaataaaacggtcatttcatcctctctccatgtgccacatgcaacacgtgcaattaaaaaatgcaggaaagaagaatacatgtccaccattaactcctgtatatgatgatcagaaaccaatgcatgttgatgcaggtcccaacctgataggaatattgcttagtcttacaatagctaaagatgtaattagcggccctccacctaacaatgcagtcaaaaggagcgagaactagtgatggaccaagaatttttatttaggtgggctaaacttttattcaagtctgctaactttattcacattaaaataccaaatgattgtgcacttttccatctcaaatcttttgcttaattgaagggtcttcccattgcctcttccaaatttaggtaatcatcaactcatcatcatcaaaacaactaagatcacatttcttataatcctagttgtttgggtgagcatctagttacccaatttgggttcccaaagggattctatttcaatctaagaaacttggttgcttgtacttacataactataaaaataattttctatagttggtcatgggcttatggcaaagaagaaaaatcgtcacttaaatataacttaaattatccaaacatatgaaccctaaggcgtggtttatttgtgaattgaaaattaaaatgtgccacaatggtgatttggtaatcttcggacttgtaaatatgcttagactaggctccaccgcccaccttcatgtgtatatatatttattgggggtcttctcatgtatagttccctcatcatatggcaaggaacacttcctcttttggagattttcaagtacatagggtgaggtacacttgtacttgaaaatctccaaaatgagaagtgtccctctccataccccctatttatctataccatcaatatacgtaacaactaggcatatacggatgaggaaggatatggcattaataaataaaagttacgttttatttattttaatgcaacgtcaataaatgagggagactgctattagtcttccatcacatggtataaaaaattccaaccctttgtcactcttggagccttggtatgctgtatgattgtcatcttttgcacatgaccaccgtctatcttgtaaagtctatccaccattgcaatccactctatgtctttttatcctactgtgcgggtgatctagtaaatcacctgtcatgattctttcataagcgatcattaaagtggcttaatcaagaggttgagatcatgaggtttgaatgagtgttcatgaatgaggaaaagaggatgttctggttttattcaagagaaagaaagtaatagtgatagtggatcgttgcaaatgcataaaagatgggggcaatggttgaattttgatatcataaaaacttttcacattggaataaaaccactaatcttttattaacacaaacatcctaatttatatgatagaagaggaataacatgaaagtaatttaaaattcatactcgtgattacattcataacgtctttgacaaactaatccaaatatcttgtaaggcccttccatattgctactctcctatctatcatcggcacaatctcaaccaaagttggctcattgatggctataaatctactcctgaatcaatgttagcccattgtttgattttgatgaatgttttttccatacaacatcataaaacaattctttaacggggattattaatgaataatgacaccgtttttaaactggccagaaaagaaaagataaaaataagcccaccagcatgtatttaacaaaacacatgttataataacagtcaacatgcccttcgagtctgatgcattttcccgcagcccatcgtttctatatatatattatatcaatacgccatatgcacgtgaagacatagagatgtttttatttcacccttaaaaacaacaacccgacagtaatcaatgagacaggccacgccacatggtgtcatccatcacgtggtcgaaaaaagatattcataaaaaaatccatccagatccgaacactggcctatttaaactcgatcttttctccccatctcaatcacccaataccaacacaagaaaacaacacacaaagtctctgctccctttccctgttcctcggattccgaaagatgatgtctgttcatcatcccacaacaaccccactttcaacttcatccccccacaaaaaacaccaatccaaaagactccaccaaaagcacccagctgtttatacaagcagaagaacagcagggtggtgcactggtattagaagtagtagtaacaacaagggtgagaatgctggtgaagagaagtctgcttcttcaaaaaggatcgaccggcgagaagtgctcctcggcctgggaggactttatggggcagctggtctcgccggccaggcccttgcttcgccggtggggatcccagaccggactgcctgcggcgatgccagctccgcaaacatctcggggccactgaagtgctgccccccagagaaagtaactactgctccgattgtccagtggaaggctcccagtccgggtcctctccgggtcaggaaaccggcacatgagatgaacaaggacgaggtggccaagttcaaaaaggcagtgcaggcgatgaaagatctggatccggaggacccatggcactatgaccagcaggcgaaaattcactgcacctactgcaacggggcttacaagcaggctggcttcgacgtccctctccaggtccacttcagctggctcttcctcccttggcaccgttggtacctctacttctttgagaggatactggggaagctgatcaacgacgatagcttcgctctcccattctggaactatgacaggccagaggggatgtttatgcccagcatatacgtcgatccctcctcgtctctctacaaccccagacggaacctggatcatctcgaaatgctgctggattacaacttcagctacgacgtgaaaggcttgacggggacggagaaggaggtcattcaggccaacctggtcgacctgcggaccatgtacgacagtggcattcccacgccagagctgttcatgggtgacccgctatccgcaggtgagctaaccgcagaggacaactcgtctggtgcgctcgagaggttccacaacacggtgcacatgtgggttgggagacacaaggacgcgacccccgacccctacatcgacatgggagacttctccaccgcggctaaggacatgctcttctacggtcaccatgccaatgtggatcgcttgtgggatatctaccggacggccagaggaaagaaggtggaattcaacaatagcgactggctcaatgcggagttcatcttctacgacgagaataaacaggtggtcaaagtcaacgtgaaggacactctaagtacacaagatctggggtacacctataaggatgttcctattccatggatgcaacgtgcacctcctaaaagaccagcggctaagcccaggtccgggtccttctctatggtccctgtgaccgagtttgggaccgaacccaaatcgctcgttgaagggcccgtccgggtcttggtcacgaggccgaagaccggccgtagccaggaggagaaggaggatgagaacgaagtcctcgtcgttgatggaattgaagtcttagatgaagggccagtcaggtttgatgtgtttattaccaccccgtttgggacgtttgcaggacccgactatgggttgcctgcagggagcttcgtgaagctgcctcatagacacaaggaagggcacaagcataggaaggcgaagctgaagctggggattacgaggctgttggaggacctcaaagctgagaatgcgcagaagctggttgtgaccctggttcctcgaactgggaaagtgaacgttggagggattcatgtggagcacttcaagactgataattag

SEQ ID NO: 23 - DNA, 3480 bp, Persea americana
atgtctcttcatcatctaacgaccaccaccccactttcaacttcatccccccaccaaaaaactcaattccaaaagcttgacaaaaagcacttatctgtttatacaagcagaaggacaacagggtggccaagtagtattagaagtagtagtactaacagcaatggtgatgagactattgctggtgaagagcaatctgcttcttcgaaacgggtcgaccggcgagacgtcctactcggcctgggagggctgtatggtgcagctggtctcgccggccaggccctggcgtcgccggtgaccatccccgaccggaatgcctgcggcattgcaacgtccccagtactccccgggccaatttattgctgcccaccagagaaagtaaggactgctcctatcgtccagtggcagtcttccaacaagggtccactccgggtcaggaaaccggcccaggagatgaacaaggacgaggtggccaggttcaaagcggcagtgcaggctatgaaagatctggatccggaggacccatggcacttcgaccagcaggcgaaaatccactgcgcctactgtaacggggcttacaagcaggtgggcttcgacgtccctcttcaggtccacttcagctggctcttcctcccttggcaccgctggtacctctacttctttgagaggatacttggaaagctgatccaggacgagagctttgctctcccattctggaactatgacaggccggaggggatgtatatgccgagcatctacgtcgacccatcctcgtccctctacaactccaaaaggaacccgaaacatctggaatcgctcctggatttcaattacagctacgatgcagatggcttaacggggacggagaaggaggtcattcaggcaaacctagtggaactgcggaccatgtacgacagtggcattcccacgccagagctgttcatgggtgacccggtttccgcaggagaggagaccgcggaggacaactcgtccggctcgctcgagaggttccacaacacggtgcacatgtgggttgggagacacaagaatgatgcgaccgagccctacgtggacatgggtgacttctccaccgcggctaaggacatgctcttctacgcgcaccattccaatgtggaccgcttgtgggaaatctacaggacgcgccgagggaagaagttggagttcaaaagcaacgactggctcaatgccgagttcatcttcttcgacgagaatagacaggtggtcaaagtaaacgtgaacgattctctaagtacactcgatctggggtacacctataaggatagtgttcccactccatggttggaacctgcacgtcctaaaagaccagcagctaagcctaggtccgggtccttctctatggtccctgtgaccgagtttgggactgaacccagagctctcgtggacgcgcctgtccgggtcttggtctccagaccgaagactagccgtagccaggatgagaaggaggacgagaacgaagtcctcgttgtcgatggaattgaagtggtagaggaaggggctgtcaggtttgatgtctttctcacctccccgtttgggaactttgcaggacccgactatgggttgcctgcagggagcttcgtgaagctgccccataaacataaggcagggagcaagcagaggaaggcgaagctgaagctggggattacgaagctgttggaggacctcaaagctgataacgcgcagaagctggttgtgacctttgtgccccggactgggagtgtgaacattggaggagtacatcacccagctgtttatacaagcagaagaacagcagggtggtgcactggtattagaagtagtagtaacaacaagggtgagaatgctggtgaagagaagtctgcttcttcaaaaaggatcgaccggcgagaagtgctcctcggcctgggaggactttatggggcagctggtctcgccggccaggcccttgcttcgccggtggggatcccagaccggactgcctgcggcgatgccagctccgcaaacatctcggggccactgaagtgctgccccccagagaaagtaactactgctccgattgtccagtggaaggctcccagtccgggtcctctccgggtcaggaaaccggcacatgagatgaacaaggacgaggtggccaagttcaaaaaggcagtgcaggcgatgaaagatctggatccggaggacccatggcactatgaccagcaggcgaaaattcactgcacctactgcaacggggcttacaagcaggctggcttcgacgtccctctccaggtccacttcagctggctcttcctcccttggcaccgttggtacctctacttctttgagaggatactggggaagctgatcaacgacgatagcttcgctctcccattctggaactatgacaggccagaggggatgtttatgcccagcatatacgtcgatccctcctcgtctctctacaaccccagacggaacctggatcatctcgaaatgctgctggattacaacttcagctacgacgtgaaaggcttgacggggacggagaaggaggtcattcaggccaacctggtcgacctgcggaccatgtacgacagtggcattcccacgccagagctgttcatgggtgacccgctatccgcaggtgagctaaccgcagaggacaactcgtctggtgcgctcgagaggttccacaacacggtgcacatgtgggttgggagacacaaggacgcgacccccgacccctacatcgacatgggagacttctccaccgcggctaaggacatgctcttctacggtcaccatgccaatgtggatcgcttgtgggatatctaccggacggccagaggaaagaaggtggaattcaacaatagcgactggctcaatgcggagttcatcttctacgacgagaataaacaggtggtcaaagtcaacgtgaaggacactctaagtacacaagatctggggtacacctataaggatgttcctattccatggatgcaacgtgcacctcctaaaagaccagcggctaagcccaggtccgggtccttctctatggtccctgtgaccgagtttgggaccgaacccaaatcgctcgttgaagggcccgtccgggtcttggtcacgaggccgaagaccggccgtagccaggaggagaaggaggatgagaacgaagtcctcgtcgttgatggaattgaagtcttagatgaagggccagtcaggtttgatgtgtttattaccaccccgtttgggacgtttgcaggacccgactatgggttgcctgcagggagcttcgtgaagctgcctcatagacacaaggaagggcacaagcataggaaggcgaagctgaagctggggattacgaggctgttggaggacctcaaagctgagaatgcgcagaagctggttgtgaccctggttcctcgaactgggaaagtgaacgttggagggattcatgtggagcacttcaagactgataattag

SEQ ID NO: 24 - protein, 1159 aa, Persea americana
MSLHHLTTTTPLSTSSPHQKTQFQKLDKKHLSVYTSRRTTGWPSSIRSSSTNSNGDETIAGEEQSASSKRVDRRDVLLGLGGLYGAAGLAGQALASPVTIPDRNACGIATSPVLPGPIYCCPPEKVRTAPIVQWQSSNKGPLRVRKPAQEMNKDEVARFKAAVQAMKDLDPEDPWHFDQQAKIHCAYCNGAYKQVGFDVPLQVHFSWLFLPWHRWYLYFFERILGKLIQDESFALPFWNYDRPEGMYMPSIYVDPSSSLYNSKRNPKHLESLLDFNYSYDADGLTGTEKEVIQANLVELRTMYDSGIPTPELFMGDPVSAGEETAEDNSSGSLERFHNTVHMWVGRHKNDATEPYVDMGDFSTAAKDMLFYAHHSNVDRLWEIYRTRRGKKLEFKSNDWLNAEFIFFDENRQVVKVNVNDSLSTLDLGYTYKDSVPTPWLEPARPKRPAAKPRSGSFSMVPVTEFGTEPRALVDAPVRVLVSRPKTSRSQDEKEDENEVLVVDGIEVVEEGAVRFDVFLTSPFGNFAGPDYGLPAGSFVKLPHKHKAGSKQRKAKLKLGITKLLEDLKADNAQKLVVTFVPRTGSVNIGGVHHPAVYTSRRTAGWCTGIRSSSNNKGENAGEEKSASSKRIDRREVLLGLGGLYGAAGLAGQALASPVGIPDRTACGDASSANISGPLKCCPPEKVTTAPIVQWKAPSPGPLRVRKPAHEMNKDEVAKFKKAVQAMKDLDPEDPWHYDQQAKIHCTYCNGAYKQAGFDVPLQVHFSWLFLPWHRWYLYFFERILGKLINDDSFALPFWNYDRPEGMFMPSIYVDPSSSLYNPRRNLDHLEMLLDYNFSYDVKGLTGTEKEVIQANLVDLRTMYDSGIPTPELFMGDPLSAGELTAEDNSSGALERFHNTVHMWVGRHKDATPDPYIDMGDFSTAAKDMLFYGHHANVDRLWDIYRTARGKKVEFNNSDWLNAEFIFYDENKQVVKVNVKDTLSTQDLGYTYKDVPIPWMQRAPPKRPAAKPRSGSFSMVPVTEFGTEPKSLVEGPVRVLVTRPKTGRSQEEKEDENEVLVVDGIEVLDEGPVRFDVFITTPFGTFAGPDYGLPAGSFVKLPHRHKEGHKHRKAKLKLGITRLLEDLKAENAQKLVVTLVPRTGKVNVGGIHVEHFKTDN

SEQ ID NO: 25 - RNA, 100 nt, Artificial
  gRNA
ggugcaucccaguuccagaaguuuuagagcuagaaauagcaaguuaaaauaaggcuaguccguuaucaacuugaaaaaguggcaccgagucggugcuuuu

SEQ ID NO: 26 - DNA, 20 bp, Persea americana
ggtgcatcccagttccagaa

SEQ ID NO: 27 - DNA, 20 bp, Persea americana
ggggcgtcccagttccagaa

SEQ ID NO: 28 - DNA, 23 bp, Persea americana
ggtgcatcccagttccagaaggg

SEQ ID NO: 29 - DNA, 20 bp, Persea americana
ggtgcatcccagttgaaggg

SEQ ID NO: 30 - DNA, 17 bp, Persea americana
ggtgcatcccagaaggg

SEQ ID NO: 31 - DNA, 11 bp, Persea americana
ggtgcgaaggg

SEQ ID NO: 32 - DNA, 24 bp, Persea americana
ggtgcatcccagttccaagaaggg

SEQ ID NO: 33 - DNA, 22 bp, Persea americana
ggtgcatcccagttccgaaggg

SEQ ID NO: 34 - DNA, 22 bp, Persea americana
ggtgcatcccagttccaaaggg

SEQ ID NO: 35 - DNA, 21 bp, Persea americana
ggtgcatcccagttcgaaggg

SEQ ID NO: 36 - DNA, 21 bp, Persea americana
ggtgcatcccagttagaaggg

SEQ ID NO: 37 - DNA, 21 bp, Persea americana
ggtgcatcccagtttgaaggg

SEQ ID NO: 38 - DNA, 19 bp, Persea americana
ggtgcatcccagagaaggg

SEQ ID NO: 39 - DNA, 19 bp, Persea americana
ggtgcatcccagtgaaggg

SEQ ID NO: 40 - DNA, 18 bp, Persea americana
ggtgcatcccaggaaggg

SEQ ID NO: 41 - DNA, 16 bp, Persea americana
ggtgcatcccagttcg

SEQ ID NO: 42 - DNA, 15 bp, Persea americana
ggtgcatcccagtgg

SEQ ID NO: 43 - DNA, 12 bp, Persea americana
ggtggataaggg

SEQ ID NO: 44 - DNA, 9 bp, Persea americana
ggagaaggg

SEQ ID NO: 45 - DNA, 9 bp, Persea americana
ggtgaaggg

SEQ ID NO: 46 - DNA, 24 bp, Persea americana
ggtgcaagcaaatgtctcatctcc

SEQ ID NO: 47 - DNA, 20 bp, Persea americana
ggtgcaaatgtctcatctcc

SEQ ID NO: 48 - DNA, 16 bp, Persea americana
ggaatgtctcatctcc

SEQ ID NO: 49 - DNA, 44 bp, Persea americana
ggtgcatcccagttccagaaggggagagcaaatgtctcatctcc

SEQ ID NO: 50 - DNA, 23 bp, Persea americana
ggggcgtcccagttccagaaggg

SEQ ID NO: 51 - DNA, 20 bp, Persea americana
ggggcgtcccagaagaaggg

SEQ ID NO: 52 - DNA, 24 bp, Persea americana
ggggcgtcccagttccaagaaggg

SEQ ID NO: 53 - DNA, 22 bp, Persea americana
ggggcgtcccagttccgaaggg

SEQ ID NO: 54 - DNA, 21 bp, Persea americana
ggggcgtcccagttcgaaggg

SEQ ID NO: 55 - DNA, 19 bp, Persea americana
ggggcgtcccagagaaggg

SEQ ID NO: 56 - DNA, 20 bp, Persea americana
ggggcgtcccagttccccaa

SEQ ID NO: 57 - DNA, 14 bp, Persea americana
ggggccatccccaa

SEQ ID NO: 58 - DNA, 13 bp, Persea americana
ggggcatccccaa

SEQ ID NO: 59 - DNA, 37 bp, Persea americana
ggggcgtcccagttcatggcgaaggactcatccccaa

SEQ ID NO: 60 - DNA, 18 bp, Persea americana
ggggcgtcccaagaaggg

SEQ ID NO: 61 - DNA, 46 bp, Persea americana
ggggcgtcccagttccagaaggggatggcgaaggactcatccccaa

SEQ ID NO: 62 - DNA, 20 bp, Artificial
  Synthetic
ttcgaacctttgatatggca

SEQ ID NO: 63 - DNA, 20 bp, Artificial
  Synthetic
attaccgaagattagccgag

SEQ ID NO: 64 - DNA, 20 bp, Artificial
  Synthetic
ttcgaacctttgatatggca

SEQ ID NO: 65 - DNA, 20 bp, Artificial
  Synthetic
agaggcactgaaaagtcaaa

SEQ ID NO: 66 - DNA, 20 bp, Artificial
  Synthetic
taactttcactctcaccagc

SEQ ID NO: 67 - DNA, 20 bp, Artificial
  Synthetic
agaggcactgaaaagtcaaa

(19) United States
(12) Patent Application Publication
(10) Pub. No.: US 2023/0265451 A1
(43) Pub. Date: Aug. 24, 2023

(54) RAPID GENERATION OF PLANTS WITH DESIRED TRAITS

(71) Applicant: GREENVENUS LLC, Davis, CA (US)

(72) Inventors: Yao LUO, Davis, CA (US); Trang DANG, Davis, CA (US); Jintai HUANG, Davis, CA (US); Sekhar BODDUPALLI, Davis, CA (US); Erinn MADDEN, Davis, CA (US); Jeffrey TOUCHMAN, Davis, CA (US); Jyoti ROUT, Davis, CA (US); Quyen LAM, Davis, CA (US); Arianne TREMBLAY, Davis, CA (US)

(21) Appl. No.: 18/020,804

(22) PCT Filed: Aug. 12, 2021
(86) PCT No.: PCT/US2021/045712

§ 371 Date: Feb. 10, 2023

Related U.S. Application Data
(60) Provisional application No. 63/064,664, filed on Aug. 12, 2020.

(51) Int. Cl. C12N 15/82

(52) U.S. Cl. CPC C12N 15/8266; C12N 15/8243

(57) ABSTRACT

The present application relates to avocado plant cells and avocado plant varieties that have combinations of polyphenol (PPO) gene loss of function mutations resulting in avocado plant cells and avocado plants with desirable traits, such as reduced browning and longer shelf life as compared to unmodified varieties. The plants and plant cells containing PPO loss of function mutations do not have any exogenous sequences in the genome. The present application also provides methods for making such plants and plant cells.

This application claims the priority benefit of U.S. Provisional Patent Application Ser. No. 63/064,664, filed Aug. 12, 2020, which is hereby incorporated by reference in its entirety.

### FIELD

The present application is directed to compositions and methods of altering traits in plants, especially avocado, including traits associated with reduced browning and increased shelf life.

### BACKGROUND

There is a need in agriculture for technology to rapidly develop unique varieties of non-GMO (“non-Genetically Modified Organism”) fruits and vegetables with superior consumer appeal and improved profitability to growers for the market. Conventionally, breeding of fruits and vegetables for selected traits is time consuming. Some fruits and vegetables are difficult to breed and develop or to genetically manipulate without introducing plant pest sequences. A platform technology to accomplish rapid, reliable, and stable genetic changes to introduce desired traits into fruits and vegetables is greatly needed. One such fruit that has proven to be difficult to introduce desired genetic changes into is the avocado (Persea americana Mill.).

Globally there are over 570 thousand hectares of avocado trees in production. In the United States alone, 2016 avocado sales were close to $2.0 B on a volume of 1.9 B units. The average consumption of avocado in the United States was projected to grow up to 50 million pounds per week in 2019 from 23 million pounds in 2014. Of this volume, over 89% is comprised of one variety, the Hass, which was introduced in 1926 and is the most widely grown in the world. According to the U.S. Department of Agriculture (USDA), avocado consumption per capita has increased 443 percent in the last 20 years from 1.6 pounds in 1995 to a record high of 7.1 pounds in 2015. Most of the avocados grown in the United States come from California, followed by Florida and Hawaii. However, production has not kept up with consumption in the U.S. Today, over 80 percent of the avocados consumed in the U.S. come from other countries, primarily from Mexico, with smaller amounts from Chile and Peru. In just 10 years, imports of avocado to the U.S. rose 41%. With increased global footprint and exports, shelf-life is a critical trait for both fresh and processed avocado products.

The rapid increase in the consumption of avocado is attributable to its healthfulness and taste profile that appeals to a broad spectrum of ethnically diverse consumers. According to 2018 consumer research published by the Hass Avocado Board, browning is a top barrier for avocado consumption, followed by rapid perishability. Browning of avocado flesh after exposure to oxygen is a significant problem for producers and consumers. In the period of time that occurs from the harvest of an avocado product to its final destination, quality and quantity losses of 5-25% can take place due to friction damage, which is characterized by an oxidation of the tissue that later inclines downward and becomes necrotic. Browning also limits the downstream use of avocados in the food service industry, because it is a major deterrence to consumers. The expected reduction in value accumulating at the grower, packing house, retailer, and consumer is estimated to be in the range of 15%.

A critical bottleneck for developing improved traits in avocado through genome editing is the lack of an efficient and reproducible regeneration system (Palomo-Rios et al., “Enhancing Frequency of Regeneration of Somatic Embryos of Avocado (Persea americana Mill.) using Semi-Permeable Cellulose Acetate Membranes,” Plant Cell Tissue and Organ Culture 115:199-207 (2013). To date, most of the successes in regenerating avocado stems from callus originating from zygotic embryo-derived tissues. Avocado plants regenerated in this manner are not true-to-type. The efficient regeneration of the Hass genotype using zygotic embryos and protoplasts has not yet been demonstrated. In addition, genome editing efficiency is variable due to the lack of effective delivery of the editing components (Zheng et al., “Profiling Single-Guide RNA Specificity Reveals a Mismatch Sensitive Core Sequence,” Sci Rep. 7(1):40638 (2017)). As a result, developing new avocado varieties with engineered traits is limited.

There is a need in the art for an efficient and reproducible system to regenerate avocado plants from true-to-type tissues to allow rapid trait manipulation and production of avocado plants with new and desirable traits.

### SUMMARY

One aspect of the present application relates to an avocado plant cell comprising a loss of function mutation of a nucleic acid sequence encoding a polyphenol oxidase of PPO-A, PPO-B, or PPO-C, where the avocado plant cell has reduced polyphenol oxidase activity.

Another aspect of the present application relates to an avocado plant comprising the avocado plant cell of the present application.

A further aspect of the present application relates to an avocado plant fruit comprising the avocado plant cell of the present application.

Another aspect of the present application relates to an avocado plant, plant part, or fruit propagated from an avocado plant or fruit of the present application.

A further aspect of the present application relates to a method of making an avocado plant cell comprising a loss of function mutation in polyphenol oxidase A (PPO-A). This method involves isolating nucellar tissue from an avocado plant, deriving a protoplast cell from the nucellar tissue, transfecting the protoplast cell with gene editing components, editing the protoplast cell genome to induce loss of function mutations in polyphenol oxidase A (PPO-A), and culturing the protoplast cell to make an avocado plant cell having a loss of function mutation in polyphenol oxidase A (PPO-A).

Another aspect of the present application relates to a method of making an avocado plant cell comprising altered expression of a gene of choice. This method involves isolating nucellar tissue from an avocado plant, deriving a protoplast cell from the isolated nucellar tissue, transfecting the protoplast cell with gene editing components to edit the protoplast cell genome to alter the expression of a gene, and culturing the protoplast cell to make an avocado plant cell comprising altered expression of the gene.

The present application relates to the development of avocado plants with improved traits, including reduced browning and increased shelf life. Described herein is the identification of at least eight putative polyphenol oxidase (“PPO”) genes in avocado. The inventors of the present application have determined which PPO gene(s) are necessary and sufficient to achieve a variety of non-browning traits in avocado plants to impart longer shelf life and to help reduce waste of cultivated avocados for human consumption. Furthermore, an efficient and reproducible system to regenerate avocado plants from true-to-type tissues has been developed to alter traits in avocado plants. As described herein, the development of an efficient and reproducible method combined with genome editing allows genetic alteration of PPO genes to impart significant phenotypic effects. Using the genetic approaches described in the present application, avocado plant cells and avocado plants are produced with loss of function mutations in specific PPO genes to significantly reduce PPO activity.

### BRIEF DESCRIPTION OF THE DRAWINGS

FIG. 1 is a graph showing the percentage of callus induction from putative avocado nucellar tissue using immature fruits of various sizes in cm from the 2018 growing season.

FIG. 2 is a graph showing the percentage of callus induction from putative nucellar tissue using immature fruits from the 2019 growing season.

FIG. 3 is a graph showing the percentage of callus induction from zygotic tissue using immature fruits from the 2019 growing season.

FIG. 4 is a graph showing the average number of nucellar somatic embryo regenerations per plate of 70 mg calli using different nucellar Haas lines of avocado.

FIG. 5 is a graph showing the germination rate of nucellar-derived somatic embryos of Haas lines N19 and N20.

FIGS. 6A-B are photographs showing elongation and rooting of nucellar-derived somatic embryos.

FIG. 7 is a schematic illustration with photographs showing one embodiment of a process and timeline for rapid generation of plants with engineered traits in avocado plants. This embodiment of the process is capable of generating ˜200 germinating somatic embryos from 1 gram of starting callus.

FIG. 8 is a graph showing Haas PPO RNA expression after fruit wounding. PPO gene candidates 1-8 are plotted by their normalized RMA expression levels (read counts) in fruit during a 24-hour wounding experiment. Each time point is the average of 3 replicates.

FIG. 9 is a graph showing Haas PPO RNA expression in leaf tissue. PPO gene candidates were plotted by read counts in leaf tissue. Three replicates were performed with standard deviation plotted.

FIGS. 10A-D are photographs of PPO activity assays of genome edited calli. Discoloration of selected edited lines is shown at time 0 (FIGS. 10A and 10B) and 22 hours (FIGS. 10C and 10D) after exposure to a caffeic acid substrate solution. Three biological replicates of each line were assayed. Photos were taken immediately after adding the substrate (FIGS. 10A and 10B) and 22 hours later (FIGS. 10C and 10D), indicated as Time 0 and Time 1, respectively.

FIG. 11 is a graph showing PPO activity in calli of the selected edited lines shown in FIGS. 10A-D. Each bar represents data of three biological replicates of the edited lines and the wild type control (WT). Data are means of three biological replicates ±SE. The average value is indicated. Bars labeled with different letters indicate they are statistically different based on student t-test (p<0.05).

FIGS. 12A-I are alignments of nucleotide sequences from avocado calli with edited PPO-A and PPO-B genes. A dash “-” indicates the position of a nucleotide deletion. A space is used to align the sequences. A bracket “[ ]” is used to denote deletions larger than the sgRNA target. Nucleotide insertions are italicized. The PAM site is highlighted in bold font. Percentage in parenthesis indicates the proportion of that sequence among all sequencing reads for that calli.

FIGS. 13A-E are alignments of nucleotide sequences from regenerated plants with an edited PPO-A gene. A “-” indicates a nucleotide deletion. A space is used to align the sequences. The PAM site is highlighted in bold font and an inserted nucleotide is italicized.

FIG. 14A is an illustration of the PPO-A protein domains including the transit peptide, the tyrosinase copper-binding domain, and the C-terminus. The position of the genome edited 1 nt insertion is indicated with an arrow. The insertion causes a premature stop codon downstream of the insertion as indicated with an arrow. FIG. 14B shows an alignment of the genome edit insertion in three plants that were genotyped and confirmed to contain a premature stop codon within the tyrosinase copper binding domain of PPO-A, encoding a truncated nonfunctional PPO-A protein. The PAM site is highlighted in bold font, the single nucleotide insertion is italicized.

FIGS. 15A-B show the PPO-A amino acid sequence (SEQ ID NO:3) and the PPO-B amino acid sequence (SEQ ID NO:6) with highly conserved residues indicated in enlarged, bold font. The six histidines that are critical for CuA (dashed box) and CuB (solid box) binding tyrosinase domain enzyme activity are underlined. The boxed regions using dotted lines are additional conserved domains identified in plant PPOs in previous studies.

FIGS. 16A-B are photographs of a PPO activity assay on 3-5 mm leaf discs from genome edited PPO-A plants at time 0 (FIG. 16A) and 18 hour after exposure to a caffeic acid substrate solution (FIG. 16B). Three biological replicates of each line were assayed. Photos were taken immediately after adding the substrate (0 hour) and 18 hours later. PPO activity is indicated by discoloration.

FIG. 17 is a graph showing PPO activity in leaves of three different homozygous PPO-A knockout (KO) plants (PPO1_1, PPO1_2, and PPO1_3). Each bar represents data of three biological replicates of the edited plants and the wildtype control (WT). Data are means of three biological replicates ±SE. The average value is labeled on each bar. Bars labeled with different letters indicates they are statistically different based on student t-test (p<0.05).

### DETAILED DESCRIPTION

All journal articles or other publications, patents, and patent applications referred to herein are expressly incorporated by reference as if each individual journal article, publication, patent, or patent application was specifically and individually indicated to be incorporated by reference. In the event of a conflict between any disclosure in the present application, compared to a disclosure incorporated by reference, the disclosure in the present application controls.

In the present application, a number of terms and abbreviations are used. The following definitions are provided and should be helpful in understanding the scope and practice of the present application.

The term “isolated” for the purposes of the present application means a biological material (e.g., nucleic acid or protein) that has been removed from its original environment (the environment in which it is naturally present). For example, a polynucleotide present in the natural state in a plant or an animal is not isolated. The same polynucleotide is “isolated” if it is separated from the adjacent nucleic acids in which it is naturally present. The term “purified” does not require the material to be present in a form exhibiting absolute purity, exclusive of the presence of other compounds. It is rather a relative definition.

A polynucleotide is in the “purified” state after purification of the starting material or of the natural material by at least one order of magnitude, 2 or 3 orders of magnitude, or 4 or 5 orders of magnitude.

A “nucleic acid” or “polynucleotide” is a polymeric compound comprised of covalently linked subunits called nucleotides. Nucleic acid includes polyribonucleic acid (RNA) and polydeoxyribonucleic acid (DNA), both of which may be single-stranded or double-stranded. DNA includes but is not limited to cDNA, genomic DNA, plasmid DNA, synthetic DNA, and semi-synthetic DNA. DNA may be linear, circular, or supercoiled.

A “nucleic acid molecule” refers to the phosphate ester polymeric form of ribonucleosides (adenosine, guanosine, uridine, or cytidine (“RNA molecules”)) or deoxyribonucleosides (deoxyadenosine, deoxyguanosine, deoxythymidine, or deoxycytidine (“DNA molecules”)), or any phosphoester anologs thereof, such as phosphorothioates and thioesters, in either single stranded form or a double-stranded helix. Double stranded DNA-DNA, DNA-RNA, and RNA-RNA helices are possible. The term nucleic acid molecule, and in particular DNA or RNA molecule, refers only to the primary and secondary structure of the molecule, and does not limit it to any particular tertiary forms. Thus, this term includes double-stranded DNA found, inter alia, in linear or circular DNA molecules (e.g., restriction fragments), plasmids, and chromosomes. In discussing the structure of particular double-stranded DNA molecules, sequences may be described herein according to the normal convention of giving only the sequence in the 5′ to 3′ direction along the non-transcribed strand of DNA (i.e., the strand having a sequence homologous to the mRNA). A “recombinant DNA molecule” is a DNA molecule that has undergone a molecular biological manipulation.

The term “fragment” when referring to a polynucleotide will be understood to mean a nucleotide sequence of reduced length relative to the reference nucleic acid and comprising, over the common portion, a nucleotide sequence identical to the reference nucleic acid. Such a nucleic acid fragment according to the present application may be, where appropriate, included in a larger polynucleotide of which it is a constituent.

As used herein, an “isolated nucleic acid fragment” is a polymer of RNA or DNA that is single- or double-stranded, optionally containing synthetic, non-natural, or altered nucleotide bases. An isolated nucleic acid fragment in the form of a polymer of DNA may be comprised of one or more segments of cDNA, genomic DNA, or synthetic DNA.

A “gene” refers to an assembly of nucleotides that encode a polypeptide, and includes cDNA and genomic DNA nucleic acids. “Gene” also refers to a nucleic acid fragment that expresses a specific functional RNA, protein, or polypeptide, optionally including regulatory sequences preceding (5′ noncoding sequences) and following (3′ non-coding sequences) the coding sequence. “Native gene” refers to a gene as found in nature with its own regulatory sequences. “Chimeric gene” refers to any gene that is not a native gene, comprising regulatory and/or coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A chimeric gene may comprise coding sequences derived from different sources and/or regulatory sequences derived from different sources. “Endogenous gene” refers to a native gene in its natural location in the genome of an organism. A “foreign” gene, “heterologous” gene, or “exogenous” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation or transfection procedure.

“Heterologous” or “exogenous” DNA refers to DNA not naturally located in the cell, or in a chromosomal site of the cell. Preferably, the exogenous DNA includes a gene or polynucleotides foreign to the cell.

“Transformation” refers to the introduction of a nucleic acid into a host organism. Host organisms containing a transformed DNA construct or DNA fragment are referred to as “transgenic” or “recombinant” organisms. “Transfection” refers to the introduction of a nucleic acid, a protein, or both into a host organism.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions.

A “promoter sequence” is a DNA regulatory region capable of binding RNA polymerase in a cell and initiating transcription of a downstream (in the 3′ direction) coding sequence. For purposes of the present application, the promoter sequence is bound at its 3′ terminus by the transcription initiation site and extends upstream (in the 5′ direction) to include the minimum number of bases or elements necessary to initiate transcription at levels detectable above background. Within the promoter sequence will be found a transcription initiation site (conveniently defined, for example, by mapping with nuclease Si), as well as protein binding domains (consensus sequences) responsible for the binding of RNA polymerase or transcription factors.

A coding sequence is “under the control” of transcriptional and translational control sequences in a cell when RNA polymerase transcribes the coding sequence into mRNA, which is then RNA spliced (if the coding sequence contains introns) and translated into the protein encoded by the coding sequence.

“Transcriptional and translational control sequences” are DNA regulatory sequences, such as promoters, enhancers, terminators, and the like, that provide for the expression of a coding sequence in a host cell. In eukaryotic cells, polyadenylation signals are control sequences.

As used herein a “protein” is a polypeptide that performs a structural or functional role in a living cell.

An “isolated polypeptide” or “isolated protein” or “isolated peptide” is a polypeptide or protein that is substantially free of those compounds that are normally associated therewith in its natural state (e.g., other proteins or polypeptides, nucleic acids, carbohydrates, lipids). “Isolated” is not meant to exclude artificial or synthetic mixtures with other compounds, or the presence of impurities which do not interfere with biological activity, and which may be present, for example, due to incomplete purification, addition of stabilizers, or compounding into a pharmaceutically acceptable preparation.

An “indel” is an insertion, a deletion, or a combination of one or more insertion(s) and deletion(s) of nucleic acid sequences as compared to a reference or wild type sequence.

A “reference sequence” means a nucleic acid or amino acid used as a comparator for another nucleic acid or amino acid, respectively, when determining sequence identity. A reference sequence can be a wildtype sequence.

“Sequence identity, “percent identity” or “% identical” refers to the exactness of a match between a reference sequence and a sequence being compared to it when optimally aligned. For example, sequence alignments and percent identity calculations may be determined using a variety of comparison methods designed to detect homologous sequences including, but not limited to, the Multalin program (Corpet, “Multiple Sequence Alignment with Hierarchical Clustering,” Nucleic Acids Res. 16:10881-90 (1988), which is hereby incorporated by reference in its entirety) or the Megalign® program of the LASERGENE® bioinformatics computing suite (DNASTAR® Inc., Madison, Wis.). Sequences may also be aligned using algorithms known in the art including, but not limited to, CLUSTAL V algorithm or the BLASTN or BLAST 2 sequence programs.

The term “about” typically encompasses a range up to 10% of a stated value.

PPO Genes in Avocado

One aspect of the present application relates to an avocado plant cell comprising a loss of function mutation of a nucleic acid sequence encoding a polyphenol oxidase of PPO-A, PPO-B, or PPO-C, where the avocado plant cell has reduced polyphenol oxidase activity.

Another aspect of the present application relates to an avocado plant comprising the avocado plant cell of the present application.

A further aspect of the present application relates to an avocado plant fruit comprising the avocado plant cell of the present application.

As described herein, mutations occurring in the polyphenol oxidase (“PPO”) genes of the avocado cell, plant part, plant, or fruit of the present application may be present in any one or more of an avocado's PPO genes. In some embodiments, the mutation(s) in the PPO gene is a human-induced mutation. In some embodiments, the avocado cell, plant part, or plant comprises a mutation in avocado PPO-A. In some embodiments, the avocado cell, plant part, plant, or fruit comprises a mutation in avocado PPO-B. In some embodiments, the avocado cell, plant part, plant, or fruit comprises a mutation in avocado PPO-C. In some embodiments, the avocado cell, plant part, plant, or fruit comprises a mutation in avocado PPO-A and PPO-B. In some embodiments, the avocado cell, plant part, plant, or fruit comprises a mutation in avocado PPO-A, PPO-B, and PPO-C. In some embodiments, the avocado cell, plant part, plant, or fruit comprises a further mutation in one or more of any of the PPO genes selected from the group consisting of avocado PPO-D, PPO-E, PPO-F, PPO-G, and PPO-H.

The nucleic acid and amino acid sequences for the PPO genes described herein as PPO-A, PPO-B, PPO-C, PPO-D, PPO-E, PPO-F, PPO-G, and PPO-H are the actual sequences determined for these genes from a certain variety of Haas avocado.

The genomic nucleotide sequence for avocado (Persea americana Mill.) PPO-A (SEQ ID NO:1), the coding nucleotide sequence PPO-A (SEQ ID NO:2), and the amino acid sequence for PPO-A (SEQ ID NO:3) are set forth in Table 1 (infra). Similarly, the genomic nucleotide sequence for avocado (Persea americana Mill.) PPO-B (SEQ ID NO:4), the coding nucleotide sequence PPO-B (SEQ ID NO:5), the amino acid sequence for PPO-B (SEQ ID NO:6), the genomic nucleotide sequence for avocado (Persea americana Mill.) PPO-C(SEQ ID NO:7), the coding nucleotide sequence PPO-C(SEQ ID NO:8), the amino acid sequence for PPO-C(SEQ ID NO:9), the genomic nucleotide sequence for avocado (Persea americana Mill.) PPO-D (SEQ ID NO:10), the coding nucleotide sequence PPO-D (SEQ ID NO:11), the amino acid sequence for PPO-D (SEQ ID NO:12), the genomic nucleotide sequence for avocado (Persea americana Mill.) PPO-E (SEQ ID NO:13), the coding nucleotide sequence PPO-E (SEQ ID NO:14), the amino acid sequence for PPO-E (SEQ ID NO:15), the genomic nucleotide sequence for avocado (Persea americana Mill.) PPO-F (SEQ ID NO:16), the coding nucleotide sequence PPO-F (SEQ ID NO:17), the amino acid sequence for PPO-F (SEQ ID NO:18), the genomic nucleotide sequence for avocado (Persea americana Mill.) PPO-G (SEQ ID NO:19), the coding nucleotide sequence PPO-G (SEQ ID NO:20), the amino acid sequence for PPO-G (SEQ ID NO:21), the genomic nucleotide sequence for avocado (Persea americana Mill.) PPO-H (SEQ ID NO:22), the coding nucleotide sequence PPO-H (SEQ ID NO:23), and the amino acid sequence for PPO-H (SEQ ID NO:24) are set forth in Table 1 (infra).

The mutations and methods of generating mutations described herein are applicable to homologues of these PPO genes from other plants, including other varieties of avocado with polyphenol oxidases having amino acid sequences that are at least 80% identical to SEQ ID NO:3. In some embodiments, the polyphenol oxidase has an amino acid sequence that is at least 80%, 83%, 85%, 90%, 93%, 95%, 98%, 99%, or 100% identical to the amino acid sequence of SEQ ID NO:3. Also encompassed are amino acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or 100% identical with the entire sequence of SEQ ID NO:3.

In some embodiments, the mutations and methods of generating mutations described herein are applicable to homologues of PPO genes described herein but from other plants, including other varieties of avocado with polyphenol oxidases having amino acid sequences that are at least 80% identical to SEQ ID NO:6. In some embodiments, the polyphenol oxidase has an amino acid sequence that is at least 80%, 83%, 85%, 90%, 93%, 95%, 98%, 99%, or 100% identical to the amino acid sequence of SEQ ID NO:6. Also encompassed are amino acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:6.

In some embodiments, the mutations and methods of generating mutations described herein are applicable to homologues of PPO genes described herein but from other plants, including other varieties of avocado with polyphenol oxidases having amino acid sequences that are at least 80% identical to SEQ ID NO:9. In some embodiments, the polyphenol oxidase has an amino acid sequence that is at least 80%, 83%, 85%, 90%, 93%, 95%, 98%, 99%, or 100% identical to the amino acid sequence of SEQ ID NO:9. Also encompassed are amino acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:9.

In some embodiments, the mutations and methods of generating mutations described herein are applicable to homologues of PPO genes described herein but from other plants, including other varieties of avocado with polyphenol oxidases having amino acid sequences that are at least 80% identical to any one of SEQ ID NO:12, SEQ ID NO:15, SEQ ID NO:18, SEQ ID NO:21, or SEQ ID NO:24. In some embodiments, the polyphenol oxidase has an amino acid sequence that is at least 80%, 83%, 85%, 90%, 93%, 95%, 98%, 99%, or 100% identical to the amino acid sequence of any one of SEQ ID NO:12, SEQ ID NO:15, SEQ ID NO:18, SEQ ID NO:21, or SEQ ID NO:24. Also encompassed are amino acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of any one of SEQ ID NO:12, SEQ ID NO:15, SEQ ID NO:18, SEQ ID NO:21, or SEQ ID NO:24.

Exemplary nucleic acid coding sequences for SEQ ID NO:3, SEQ ID NO:6, SEQ ID NO:9, SEQ ID NO:12, SEQ ID NO:15, SEQ ID NO:18, SEQ ID NO:21, and SEQ ID NO:24 are provided as SEQ ID NO:2, SEQ ID NO:5, SEQ ID NO:8, SEQ ID NO:11, SEQ ID NO:14, SEQ ID NO:17, SEQ ID NO:20, and SEQ ID NO:23, respectively.

The mutations and methods of generating mutations described herein are applicable to homologues of PPO genes described herein but from other plants, including other varieties of avocado with nucleic acid sequences that are at least 80% identical to SEQ ID NO:2. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:2.

In some embodiments, the mutations and methods of generating mutations described herein are applicable to homologues of PPO genes described herein but from other plants, including other varieties of avocado with nucleic acid sequences that are at least 80% identical to SEQ ID NO:5. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:5.

In some embodiments, the mutations and methods of generating mutations described herein are applicable to homologues of PPO genes described herein but from other plants, including other varieties of avocado with nucleic acid sequences that are at least 80% identical to SEQ ID NO:8. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:8.

In some embodiments, the mutations and methods of generating mutations described herein are applicable to homologues of PPO genes described herein but from other plants, including other varieties of avocado with nucleic acid sequences that are at least 80% identical to any one of SEQ ID NO:11, SEQ ID NO:14, SEQ ID NO:17, SEQ ID NO:20, or SEQ ID NO:23. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of any one of SEQ ID NO:11, SEQ ID NO:14, SEQ ID NO:17, SEQ ID NO:20, or SEQ ID NO:23.

Additional exemplary nucleic acid sequences encoding SEQ ID NO:3, SEQ ID NO:6, SEQ ID NO:9, SEQ ID NO:12, SEQ ID NO:15, SEQ ID NO:18, SEQ ID NO:21, and SEQ ID NO:24 are provided as SEQ ID NO:1, SEQ ID NO:4, SEQ ID NO:7, SEQ ID NO:10, SEQ ID NO:13, SEQ ID NO:16, SEQ ID NO:19, and SEQ ID NO:22, respectively.

The mutations and methods of generating mutations described herein are applicable to homologues of PPO genes described herein but from other plants, including other varieties of avocado with nucleic acid sequences that are at least 80% identical to SEQ ID NO:1. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:1.

In some embodiments, the mutations and methods of generating mutations described herein are applicable to homologues of PPO genes described herein but from other plants, including other varieties of avocado with nucleic acid sequences that are at least 80% identical to SEQ ID NO:4. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:4.

In some embodiments, the mutations and methods of generating mutations described herein are applicable to homologues of PPO genes described herein but from other plants, including other varieties of avocado with nucleic acid sequences that are at least 80% identical to SEQ ID NO:7. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of SEQ ID NO:7.

In some embodiments, the mutations and methods of generating mutations described herein are applicable to homologues of PPO genes described herein but from other plants, including other varieties of avocado with nucleic acid sequences that are at least 80% identical to any one of SEQ ID NO:10, SEQ ID NO:13, SEQ ID NO:16, SEQ ID NO:19, or SEQ ID NO:22. Also encompassed are nucleic acid sequences at least 80%, 81%, 82%, 83%, 84%, 85%, 86%, 87%, 88%, 89%, 90%, 91%, 92%, 93%, 94%, 95%, 96%, 97%, 98%, 99% identical, or could be 100% identical with the entire sequence of any one of SEQ ID NO:10, SEQ ID NO:13, SEQ ID NO:16, SEQ ID NO:19, or SEQ ID NO:22.

Modified PPO Genes in Avocado

The present application provides avocado cells, fruits, plant parts, and plants that have loss of function mutations in polyphenol oxidase (PPO) genes such that one or more cells of the avocado plant (or plant part) experience reduced browning when compared to cells of wildtype avocado cells, fruits, plant parts, and plants. As used herein, the term “avocado cell” or “avocado plant cell” includes cells, protoplasts, cell tissue cultures from which avocado plants can be regenerated, calli, clumps, and cells that are intact in avocado or parts of avocado including, but not limited to seeds, leaves, stems, roots, vegetative buds, floral buds, meristems, embryos, hypocotyls, cotyledons, endosperm, sepals, petals, pistils, carpels, stamens, anthers, microspores, pollen, pollen tubes, ovules, nucellar tissue, ovaries, and other avocado tissue or cells. In some embodiments, the avocado cell is a protoplast. In some embodiments, the protoplast is derived from avocado nucellar tissue. Nucellar tissue is derived from the nucellus, which is part of the inner structure of the ovule. Nucellar tissue is somatic tissue such that plants regenerated from nucellar tissue are considered identical to the mother plant, or “true-to-type” (see Shukla et al., “Nucellar Embryogenesis and Plantlet Regeneration in Monoembryonic and Polyembryonic Mango (Mangifera indica L.) Cultivars,” African Journal of Biotechnology 15:2814-2823 (2016), which is hereby incorporated by reference in its entirety). This is an important consideration for avocado production since zygotic avocado tissue will produce plants that have different characteristics from the mother plant.

In some embodiments, the avocado cell is modified by genome editing. In some embodiments, the avocado cell is a regenerable avocado cell. In some embodiments, an avocado plant comprises the avocado cell. In some embodiments, an avocado fruit comprises the avocado cell. In some embodiments, an avocado plant, plant part, or fruit is propagated from an avocado plant, plant part or fruit of any of the embodiments of the present application. In some embodiments the avocado cell is a Haas avocado cell. In some embodiments, the avocado plant, plant part or fruit is a Haas avocado plant, plant part or fruit.

Modifying the PPO genes in cells, plant parts, plants, and fruits, such as avocado, so that the plant possesses a PPO loss of function mutation, may be done by any method known in the art. That is, any method known in the art to make avocado cells, plant parts, plants, and fruits with PPO-A and/or PPO-B mutations is contemplated by the present application, as well as any combination of PPO-A and/or PPO-B mutations with one or more mutation in PPO-C, and/or other avocado PPO genes, such as PPO-D, PPO-E, PPO-F, PPO-G, and PPO-H.

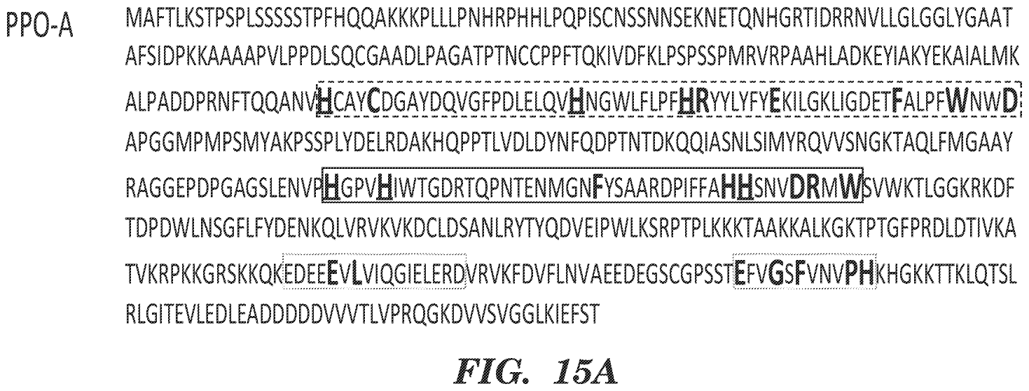

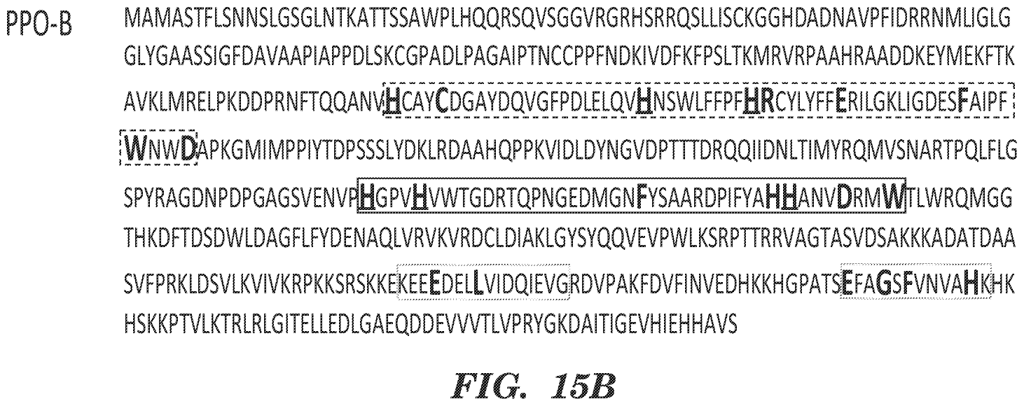

The phrase “loss of function mutation” refers to a mutation that results in a gene product no longer being able to perform its normal function, or no longer having its normal level of activity, in whole or in part, compared to a wildtype (unmutated) counterpart. Loss of function mutations are also referred to as inactivating mutations that typically result in the gene product having less or no function, i.e., being partially or wholly inactivated. Loss of function mutations include insertions and deletions that interrupt or change the coding region of a gene, such as causing a premature stop codon or altering the splicing of a nucleotide sequence. A loss of function mutation that introduces a premature stop codon is referred to herein as a “knockout” mutation. In some embodiments, the loss of function mutation is an insertion mutation. In some embodiments, the loss of function mutation is a deletion mutation. In some embodiments, the loss of function mutation is a combination of one or more insertion mutations. In some embodiments, the loss of function mutation is a combination of one or more deletion mutations. In some embodiments, the loss of function mutation is a combination of one or more insertion mutations and one or more deletion mutations. Loss of function mutations may alter the reading frame of a nucleic acid sequence, regardless of whether it is an insertion, deletion, or a combination of an insertion and deletion. For example, an insertion or deletion of 1, 2, 4, 5, 7, 8, 10, 11, 13, 14, 16, 17, 19, 20, or more than 20 nucleotides would alter the reading frame, or alter it sufficiently to provide a loss of function. (Insertions or deletions of 3, 6, 9, 12, 15, or 18 nucleotides would not alter the reading frame, however insertions or deletions larger than 21 nucleotides are expected to provide a loss of function.) In some embodiments, the polypeptides or proteins according to this or any other embodiment described herein comprise one or more (e.g., 1, 2, 3, 4, 5 or more) amino acid insertions, deletions, or other modifications (e.g., substitution of one amino acid for another) compared to a wild type sequence. In some embodiments, the loss of function mutation disrupts the key structural domain of polyphenol oxidase, namely the copper binding tyrosinase domains as indicated in FIGS. 15A-B for PPO-A (FIG. 15A) and PPO-B (FIG. 15B). Altering the reading frame of the protein with an insertion or deletion (or combination thereof) that leads to a premature stop codon is one means of altering the tyrosinase domain. Other mutations that do not change the reading frame (such as a missense mutation), but alter essential amino acids in the tyrosinase domain are considered loss of function mutations. For example, mutations in the amino acids indicated in FIGS. 15-A-B in enlarged, bold font in which the amino acid is altered to another amino acid are considered loss of function mutations.

In some embodiments, the same mutation in a PPO gene occurs in both chromosomal alleles of that PPO gene. In other words, the mutation is a homozygous mutation. In other embodiments, the mutation in a PPO gene occurs in only one chromosomal allele of that PPO gene. In other words, the mutation is a heterozygous mutation. In yet another embodiment, two different mutations in a PPO gene can occur in each chromosomal allele of the PPO gene such that both alleles comprise different mutations in the PPO gene. In some embodiments, the cell has a loss of function mutation in both chromosomal alleles of the nucleic acid sequence encoding the polyphenol oxidase.

In some embodiments, the avocado cell comprises a loss of function mutation in the nucleic acid sequence encoding the polyphenol oxidase of PPO-A. In some embodiments, the avocado cell comprises a loss of function mutation in the nucleic acid sequence encoding the polyphenol oxidase of PPO-B. In some embodiments, the avocado cell comprises a loss of function mutation in the nucleic acid sequence encoding the polyphenol oxidase of PPO-C.

In some embodiments, the avocado cell comprises a first loss of function mutation in the nucleic acid sequence encoding the polyphenol oxidase of PPO-A and a second loss of function mutation in the nucleic acid sequence encoding the polyphenol oxidase of PPO-B. In some embodiments, the first loss of function mutation comprises both alleles of the nucleic acid sequence encoding PPO-A. In some embodiments, the second loss of function mutation comprises both alleles of the nucleic acid sequence encoding PPO-B. In further embodiments, the first loss of function mutation comprises both alleles of the nucleic acid sequence encoding the polyphenol oxidase of PPO-A, and the second loss of function mutation comprises both alleles of the nucleic acid sequence encoding the polyphenol oxidase of PPO-B. In additional embodiments, the avocado cell further comprises an at least third loss of function mutation of the nucleic acid sequence encoding the polyphenol oxidase selected from any one or more of the group consisting of PPO-C, PPO-D, PPO-E, PPO-F, PPO-G, and PPO-H. In some embodiments, the third loss of function mutation comprises both alleles of the nucleic acid sequence encoding a PPO selected from the group consisting of PPO-C, PPO-D, PPO-E, PPO-F, PPO-G, and PPO-H. In some embodiments, the avocado cell comprises additional loss of function mutation(s) of the nucleic acid sequence encoding an additional one or more avocado PPO gene(s). In some embodiments, the loss of function mutation comprises both alleles of the nucleic acid sequence encoding the additional one or more polyphenol oxidase(s).

An avocado cell, plant, plant part, or fruit may contain combinations of genotypes of different PPO genes. For example, an avocado cell, plant, plant part, or fruit may have homozygous PPO gene mutations of some PPO genes, heterozygous PPO gene mutations of some PPO genes, different PPO mutations in the same gene of some PPO genes, or wild type PPO genes of some PPO genes, or any combination thereof.

In some embodiments, a mutation may be induced by treatment with a mutagenic agent. Any suitable mutagenic agent can be used for embodiments of the present application. For example, mutagens creating point mutations, deletions, insertions, rearrangements, transversions, transitions, or any combination thereof may be used. Suitable radiation mutagens include, without limitation, ultraviolet light, x-rays, gamma rays, and fast neutrons. Suitable chemical mutagens include, but are not limited to, ethyl methanesulfonate (“EMS”), methylmethane sulfonate (“MMS”), N-ethyl-N-nitrosourea (“ENU”), triethylmelamine (“TEM”), N-methyl-N-nitrosourea (“MNU”), procarbazine, chlorambucil, cyclophosphamide, diethyl sulfate, acrylamide monomer, melphalan, nitrogen mustard, vincristine, dimethylnitrosamine, N-methyl-N′-nitro-nitrosoguanidine 25 (“MNNG”), nitrosoguanidine, 2-aminopurine, 7, 12 dimethyl-benz(a)anthracene (“DMBA”), ethylene oxide, hexamethylphosphoramide, bisulfan, diepoxyalkanes (diepoxyoctane (“DEO”), diepoxybutane (“DEB”), 2-methoxy-6-chloro-9[3-(ethyl-2-chloro-ethyl) aminopropylamino] acridine dihydrochloride (“ICR-170”), sodium azide, formaldehyde, or combinations thereof.

A mutation may be detected using a method such as “TILLING” or “Targeting Induced Local Lesions in Genomes” which is a general reverse genetic method providing an allelic series of induced mutation by random chemical or physical mutagenesis, that can be used to identify mutations in a gene or region of interest. In a common use of the TILLING methodology, plant material, such as seeds, are subjected to chemical mutagenesis, which creates a series of mutations within the genomes of the seeds' cells. The mutagenized seeds are grown into adult M1 plants and self-pollinated. DNA samples from the resulting M2 plants are pooled and are then screened for mutations in a gene of interest. Once a mutation is identified in a gene of interest, the seeds of the M2 plant carrying that mutation are grown into adult M3 plants and screened for the phenotypic characteristics associated with the gene of interest. See for example, Colbert et al., “High-Throughput Screening for Induced Point Mutations,” Plant Physiology 126:480-484 (2001) and Krasileva et al., “Uncovering Hidden Variation in Polyploid Wheat,” Proc. Nat. Acad. Sci. 114-E913-E921 (2017), each of which is hereby incorporated by reference in its entirety.

In some embodiments, a mutation may be induced by genome editing. Genome editing is a type of genetic engineering in which DNA is inserted, replaced, or removed, or any combination thereof, from a genome using artificially engineered nucleases, or “molecular scissors.” The nucleases typically create double-stranded breaks (“DSBs”) at desired locations in the genome, and harness the cell's endogenous mechanisms to repair the induced break by processes of homology dependent repair (“HDR”) or nonhomologous end-joining (“NHEJ”). Any method of genome editing may be used in the embodiments of the present application.

CRISPR/Cas type RNA-guided endonucleases provide an efficient system for inducing genetic modifications in genomes of many organisms. Non-limiting examples of genome editing nucleases include Cas1, Cas1B, Cas2, Cas3, Cas4, Cas5, Cash, Cas7, Cas8, Cas9 (also known as Csn1 and Csx12), Cas10, Cas12a (Cpf1), Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, CasX, CasY, Mad7, SynNuc1, or homologs, modified versions, and endonuclease inactive versions thereof. An example of a fusion protein to Cas9 is a cytidine deaminase-Cas9 fusion protein used in cytidine base editing to mutate nucleotides in target genes without generating double-strand breaks as described in Komor et al., “Programmable Editing of a Target Base in Genomic DNA without Double-Stranded DNA Cleavage,” Nature 533:420-424 (2016), which is hereby incorporated by reference in its entirety. The use of CRISPR guide RNA in conjunction with CRISPR/Cas technology to target RNA is also described in Wiedenheft et al., “RNA-Guided Genetic Silencing Systems in Bacteria and Archaea,” Nature 482:331-338 (2012); Zhang et al., “Multiplex Genome Engineering Using CRISPR/Cas Systems,” Science 339:819-23 (2013); and Gaj et al., “ZFN, TALEN, and CRISPR/Cas-based Methods for Genome Engineering,” Cell 31:397-405 (2013), each of which is hereby incorporated by reference in their entirety.

There are typically two distinct components to a CRISPR system, a guide RNA (“gRNA”) and a genome editing endonuclease. The gRNA uses a CRISPR RNA (“crRNA”) comprising a DNA targeting segment that can be engineered to contain a complementary stretch of nucleotide sequence (e.g., at least 10 nucleotides) to target a DNA site for binding and subsequent modification by CRISPR genome editing nuclease. The length of a crRNA may range from about 15 nucleotides to about 60 nucleotides. The crRNA can be chemically synthesized and can also be engineered to include a ribonucleotide analog or a modified form thereof, or an analog of a modified form, or non-natural nucleosides.

Depending on the genome editing nuclease used, the gRNA can also comprise a trans-activating crRNA (“tracrRNA”). Such is the case with Cas9, for example. The tracrRNA is a small RNA sequence that forms a binding handle used by the CRISPR protein. The tracrRNA can be chemically synthesized and can also be engineered to include a ribonucleotide analog or a modified form thereof, or an analog of a modified form, or non-natural nucleosides.

A single guide RNA″ (“sgRNA”) combines the targeting specificity of the crRNA with the scaffolding properties of the tracrRNA into a single transcript. In the sgRNA, crRNA and tracrRNA are present either in their native form, or a modified form. The sgRNA may be about 60 nucleotides to about 120 nucleotides long. The sgRNA can be chemically synthesized and can also be engineered to include a ribonucleotide analog or a modified form thereof, or an analog of a modified form, or non-natural nucleosides. In some embodiments, the sgRNA is SEQ ID NO:25.

When the gRNA and the gene editing endonuclease are introduced into the cell, the genomic target sequence can be modified or permanently disrupted to create a loss of function mutations. A complex of a genome editing nuclease with a gRNA is called a ribonucleotide particle or ribonucleoprotein (RNP) complex. The RNP complex is recruited to the target sequence by the base-pairing between the gRNA sequence, which has a region of complementarity to the target sequence in the genomic DNA. In some embodiments, the target sequence is SEQ ID NO:26 or SEQ ID NO:27.

For successful binding of Cas9, the genomic target sequence must also contain the correct Protospacer Adjacent Motif (“PAM”) sequence immediately following the target sequence. The binding of the RNP complex localizes the genome editing nuclease to the genomic target sequence so that the genome editing nuclease can cut both strands of DNA causing a DSB. Cas9 generates DSBs through the combined activity of two nuclease domains, RuvC and HNH. Cas9 will cut 3-4 nucleotides upstream of the PAM sequence. CRISPR specificity can be controlled by level of homology and binding strength of the specific gRNA for a given gene target, or by modification of the Cas endonuclease itself. For example, a D10A mutant of the RuvC domain, retains only the HNH domain and generates a DNA nick rather than a DSB.

A software tool can be used to optimize the choice of gRNA within a target sequence, and to minimize total off-target activity across the rest of the genome. The cleavage efficiency at each off-target sequence can be estimated, e.g., using an experimentally-derived weighting scheme. Each possible gRNA is then ranked according to its total predicted off-target cleavage; the top-ranked gRNAs represent those that are likely to have the greatest on-target and the least off-target cleavage. An exemplary software tool to use for estimating gRNA cleavage efficiency is Geneious software (Geneious, San Diego, Calif.).

Other nucleases can also be used for genome editing. ZFNs are artificial restriction enzymes generated by fusing a zinc finger DNA-binding domain to a DNA-cleavage domain. Zinc finger domains can be engineered to target specific desired DNA sequences and this enables zinc-finger nucleases to target unique sequences within complex genomes. By taking advantage of endogenous DNA repair machinery, these reagents can be used to precisely alter the genomes of higher organisms. ZFNs include an engineered zinc finger DNA-binding domain fused to the cleavage domain of the FokI restriction endonuclease. ZFNs can be used to induce double-stranded breaks (DSBs) in specific DNA sequences.

TALEN is a sequence-specific endonuclease that includes a transcription activator-like effector (“TALE”) and a FokI endonuclease. The transcription activator-like effector is a DNA binding protein that has a highly conserved central region with tandem repeat units of 34 amino acids. The base preference for each repeat unit is determined by two amino acid residues called the repeat-variable di-residue, which recognizes one specific nucleotide in the target DNA. Arrays of DNA-binding repeat units can be customized for targeting specific DNA sequences. As with ZFNs, dimerization of two TALENs on targeted specific sequences in a genome results in Fold-dependent introduction of double stranded breaks, stimulating homology directed repair (“HDR”) and non-homologous end joining (NHEJ) repair mechanism.

Meganucleases with re-engineered homing nucleases can also be used to effect genome modification in plants in the methods described herein. Meganucleases are endodeoxyribonucleases characterized by a large recognition site (double-stranded DNA sequences of 12 to 40 base pairs). This site generally occurs only once in any given genome. For example, the 18-base pair sequence recognized by the I-Scel meganuclease would on average require a genome twenty times the size of the human genome to be found once by chance. Meganucleases are considered to be the most specific naturally occurring restriction enzymes. Among meganucleases, the LAGLIDADG family of homing endonucleases has become a valuable tool for the study of genomes and genome engineering over the past fifteen years. By modifying their recognition sequence through protein engineering, the targeted sequence can be changed.

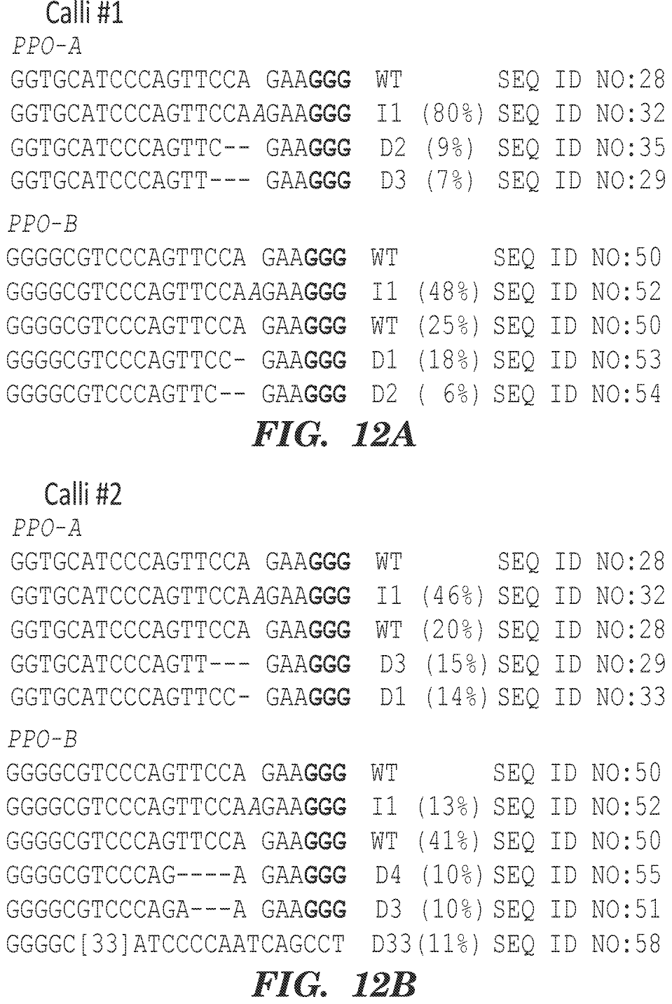

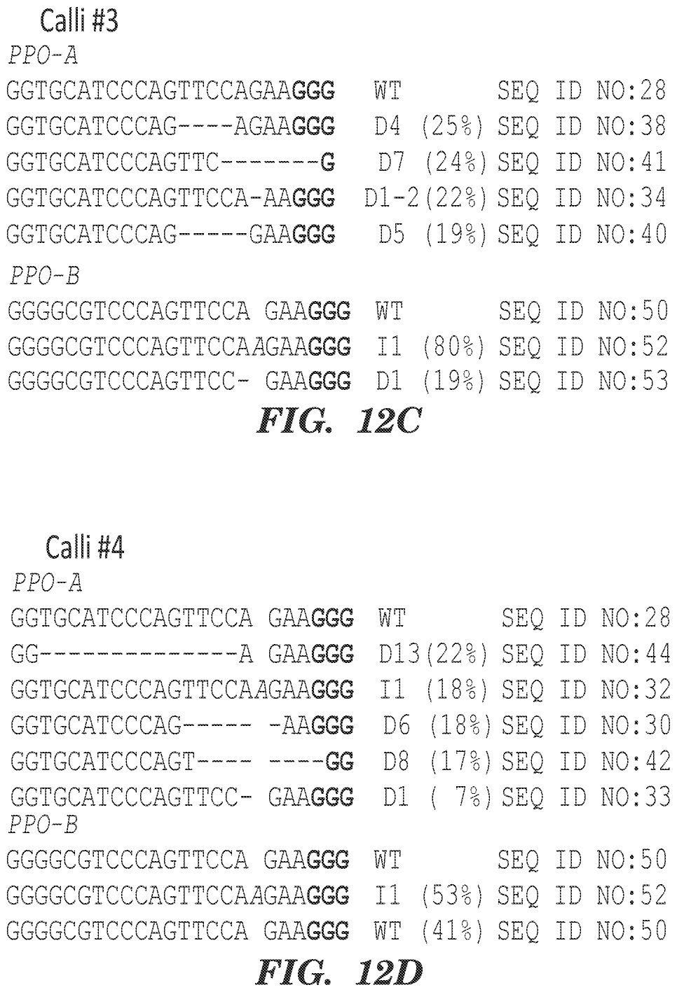

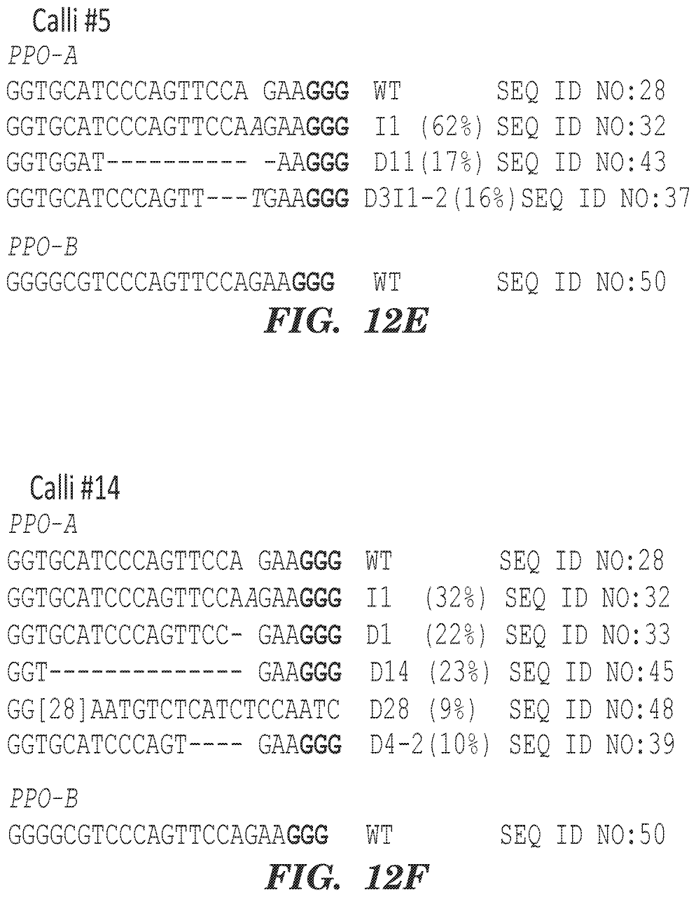

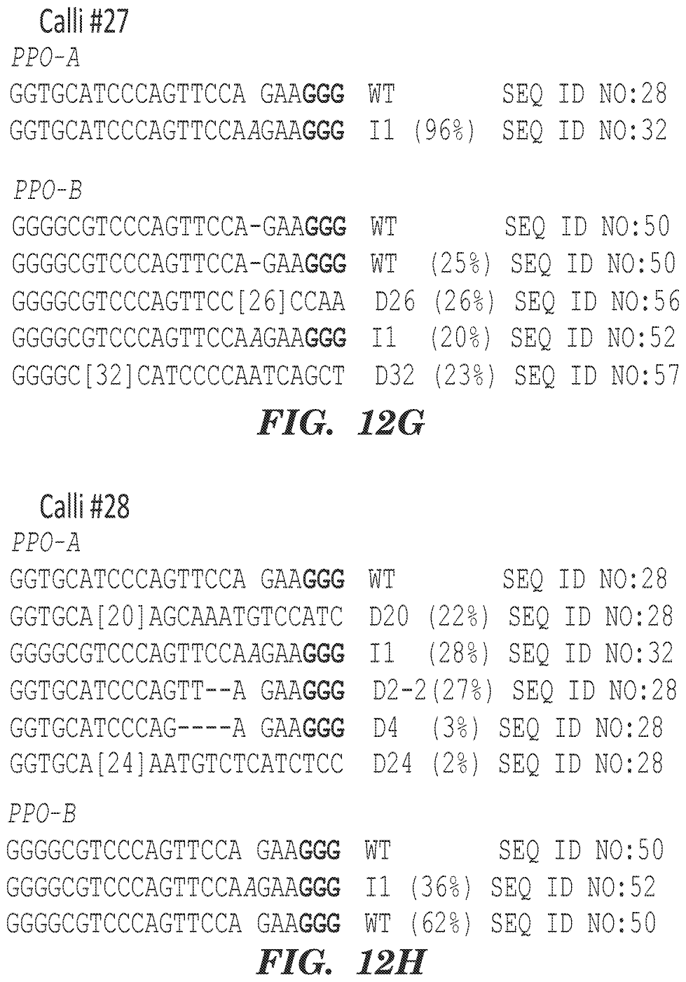

Exemplary genome edited mutations of avocado PPO-A and PPO-B genes are shown in Table 4 (infra) and FIGS. 12A-I and 13A-E.

In some embodiments, the loss of function mutation is selected from the group consisting of SEQ ID NO:32-48. In other embodiments, the loss of function mutation is selected from the group consisting of SEQ ID NO:52-60. In further embodiments, the first loss of function mutation is selected from the group consisting of SEQ ID NO:32-48, and the second loss of function mutation is selected from the group consisting of SEQ ID NO:52-60. Additional loss of function mutations are contemplated, especially if alternate sgRNAs or gRNAs were directed to different target sequences within the PPO genes, such as the PPO-A and PPO-B genes, and other avocado PPO genes, without limitation.

Avocado with Reduced PPO Activity

Additional embodiments of the present application are directed to avocado cells, plants, plant parts and fruits with reduced PPO activity. PPO protein “activity” or “PPO activity” refers to the enzymatic activity of the PPO protein(s). PPO protein activity may be measured biochemically by methods known in the art including, but not limited to, the detection of products formed by the enzyme in the presence of any number of heterologous substrates, for example, catechol and caffeic acid. PPO protein activity may also be measured functionally, for example, by assessing its effects on phenotypic traits of an avocado cell, plant, plant part, or fruit, such as fruit or leaf browning when cut or bruised.

In one embodiment, the avocado cell, plant, plant part, or fruit of the present application has a reduced activity of PPO that is 95% or less of the activity of PPO in wild type avocado cells, plants, plant parts, or fruits. In some embodiments, the avocado cell, plant, plant part, or fruit has a reduced activity of PPO that is 90% or less of the activity of PPO in wild type avocado cells, plants, plant parts, or fruits. In some embodiments, the avocado cell, plant, plant part, or fruit has a reduced activity of PPO that is 80% or less of the activity of PPO in wild type avocado cells, plants, plant parts, or fruits. In some embodiments, the avocado cell, plant, plant part, or fruit has a reduced activity of PPO that is 70% or less of the activity of PPO in wild type avocado cells, plants, plant parts, or fruits. In some embodiments, the avocado cell, plant, plant part, or fruit has a reduced activity of PPO that is 60% or less of the activity of PPO in wild type avocado cells, plants, plant parts, or fruits. In some embodiments, the avocado cell, plant, plant part, or fruit has a reduced activity of PPO that is 50% or less of the activity of PPO in wild type avocado cells, plants, plant parts, or fruits. In some embodiments, the avocado cell, plant, plant part, or fruit has a reduced activity of PPO that is 40% or less of the activity of PPO in wild type avocado cells, plants, plant parts, or fruits. In some embodiments, the avocado cell, plant, plant part, or fruit has a reduced activity of PPO that is 30% or less of the activity of PPO in wild type avocado cells, plants, plant parts, or fruits. In some embodiments, the avocado cell, plant, plant part, or fruit has a reduced activity of PPO that is 20% or less of the activity of PPO in wild type avocado cells, plants, plant parts, or fruits. In some embodiments, the avocado cell, plant, plant part, or fruit has a reduced activity of PPO that is 10% or less of the activity of PPO in wild type avocado cells, plants, plant parts, or fruits. In some embodiments, the avocado cell, plant, plant part, or fruit has a reduced activity of PPO that is 5% or less, 4% or less, 3% or less, 2% or less or 1% or less or 0% of the activity of PPO in wild type avocado cells, plants, plant parts, or fruits. In some embodiments, the avocado cell, plant, plant part, or fruit has undetectable PPO activity. The reduction in PPO activity may vary depending on a number of factors including, but not limited to the tissue type, the developmental stage of a plant or plant material, the method of cultivation, the harvesting conditions, the experimental conditions, and combinations and variations thereof.

In some embodiments, the avocado cell, plant, plant part, or fruit of the present application has a reduced activity of PPO-A that is 95% or less of the activity of PPO-A in wildtype avocado cells, plants, plant parts, or fruits. In some embodiment, the avocado cell, plant, plant part, or fruit of the present application has a reduced activity of PPO-A that is 90% or less, 80% or less, 70% or less, 60% or less, 50% or less, 40% or less, 30% or less, 20% or less, 10% or less, or 5% or less of the expression of a PPO-A gene in a wildtype avocado cell, plant part, plant or fruit of the activity of PPO-A in wild type avocado cells, plants, plant parts, or fruits. In some embodiments, the avocado cell, plant, plant part, or fruit has a reduced activity of PPO-A that is 5% or less, 4% or less, 3% or less, 2% or less or 1% or less or 0% of the activity of PPO-A in wildtype avocado cells, plants, plant parts, or fruits.

In some embodiment, the avocado cell, plant, plant part, or fruit of the present application has a reduced activity of PPO-B that is 95% or less of the activity of PPO-B in wildtype avocado cells, plants, plant parts, or fruits. In some embodiment, the avocado cell, plant, plant part, or fruit of the present application has a reduced activity of PPO-B that is 90% or less, 80% or less, 70% or less, 60% or less, 50% or less, 40% or less, 30% or less, 20% or less, 10% or less, or 5% or less of the expression of a PPO-B gene in a wildtype avocado cell, plant part, plant or fruit of the activity of PPO-A in wild type avocado cells, plants, plant parts, or fruits. In some embodiments, the avocado cell, plant, plant part, or fruit has a reduced activity of PPO-B that is 5% or less, 4% or less, 3% or less, 2% or less or 1% or less or 0% of the activity of PPO-B in wildtype avocado cells, plants, plant parts, or fruits.

In some embodiment, the avocado cell, plant, plant part, or fruit of the present application has a reduced activity of PPO-C that is 95% or less of the activity of PPO-C in wildtype avocado cells, plants, plant parts, or fruits. In some embodiment, the avocado cell, plant, plant part, or fruit of the present application has a reduced activity of PPO-C that is 90% or less, 80% or less, 70% or less, 60% or less, 50% or less, 40% or less, 30% or less, 20% or less, 10% or less, or 5% or less of the expression of a PPO-C gene in a wildtype avocado cell, plant part, plant or fruit of the activity of PPO-C in wildtype avocado cells, plants, plant parts, or fruits. In some embodiments, the avocado cell, plant, plant part, or fruit has a reduced activity of PPO-C that is 5% or less, 4% or less, 3% or less, 2% or less or 1% or less or 0% of the activity of PPO-C in wildtype avocado cells, plants, plant parts, or fruits.

In some embodiment, the avocado cell, plant, plant part, or fruit of the present application has a reduced activity of any one or more of PPO-D, PPO-E, PPO-F, PPO-G, or PPO-H that is 95% or less of the activity of any one or more of PPO-D, PPO-E, PPO-F, PPO-G, or PPO-H in wildtype avocado cells, plants, plant parts, or fruits. In some embodiment, the avocado cell, plant, plant part, or fruit of the present application has a reduced activity of PPO-D, PPO-E, PPO-F, PPO-G, and/or PPO-H that is 90% or less, 80% or less, 70% or less, 60% or less, 50% or less, 40% or less, 30% or less, 20% or less, 10% or less, or 5% or less of the expression of a PPO-D, PPO-E, PPO-F, PPO-G, and/or PPO-H gene in a wildtype avocado cell, plant part, plant or fruit of the activity of PPO-D, PPO-E, PPO-F, PPO-G, and/or PPO-H in wildtype avocado cells, plants, plant parts, or fruits. In some embodiments, the avocado cell, plant, plant part, or fruit has a reduced activity of PPO-D, PPO-E, PPO-F, PPO-G, and/or PPO-H that is 5% or less, 4% or less, 3% or less, 2% or less or 1% or less or 0% of the activity of PPO-D, PPO-E, PPO-F, PPO-G, and/or PPO-H in wildtype avocado cells, plants, plant parts, or fruits.

Avocado with Reduced PPO Expression

The “expression” of a PPO gene refers to the transcription of a PPO gene. PPO gene expression levels may be measured by any means known in the art such as, without limitation, qRT-PCR (quantitative real time PCR), semi-quantitative PCR, RNA-seq, and Northern blot analysis.

In some embodiments, the expression of a PPO gene is 90% or less, 80% or less, 70% or less, 60% or less, 50% or less, 40% or less, 30% or less, 20% or less, 10% or less, or 5% or less of the expression of a PPO gene in a wildtype avocado cell, plant part, plant, or fruit. In some embodiments, the expression of a PPO gene is undetectable. In some embodiments, the expression of a PPO-A gene is 90% or less, 80% or less, 70% or less, 60% or less, 50% or less, 40% or less, 30% or less, 20% or less, 10% or less, or 5% or less of the expression of a PPO-A gene in a wildtype avocado cell, plant part, plant or fruit. In some embodiments, the expression of a PPO-A gene is undetectable. In some embodiments, the expression of a PPO-B gene is 90% or less, 80% or less, 70% or less, 60% or less, 50% or less, 40% or less, 30% or less, 20% or less, 10% or less, or 5% or less of the expression of a PPO-B gene in a wildtype avocado cell, plant part, plant, or fruit. In some embodiments, the expression of a PPO-B gene is undetectable. In some embodiments, the expression of a PPO-C gene is 90% or less, 80% or less, 70% or less, 60% or less, 50% or less, 40% or less, 30% or less, 20% or less, 10% or less, or 5% or less of the expression of a PPO-C gene in a wildtype avocado cell, plant part, plant or fruit. In some embodiments, the expression of a PPO-C gene is undetectable. In some embodiments, the expression of any one or more of a PPO-D, PPO-E, PPO-F, PPO-G, and PPO-H gene is 90% or less, 80% or less, 70% or less, 60% or less, 50% or less, 40% or less, 30% or less, 20% or less, 10% or less, or 5% or less of the expression of a PPO-D, PPO-E, PPO-F, PPO-G, and PPO-H gene in a wild type avocado cell, plant part, plant, or fruit. In some embodiments, the expression of any one or more of a PPO-D, PPO-E, PPO-F, PPO-G, and PPO-H gene is undetectable.

The “amount” or “level” of a protein refers to the level of a particular protein, for example PPO-A, which may be measured by any means known in the art such as, without limitation, Western blot analysis, ELISA, other forms of immunological detection, or mass spectrometry.

In some embodiments, the amount of a PPO protein is 90% or less, 80% or less, 70% or less, 60% or less, 50% or less, 40% or less, 30% or less, 20% or less, 10% or less, or 5% or less of the amount of a PPO protein in a wildtype avocado plant cell, plant part, plant, or fruit. In some embodiments, the amount of a PPO protein is undetectable. In some embodiments, the amount of a PPO-A protein is 90% or less, 80% or less, 70% or less, 60% or less, 50% or less, 40% or less, 30% or less, 20% or less, 10% or less, or 5% or less of the amount of a PPO-A protein in a wildtype avocado plant cell, plant part, plant, or fruit. In some embodiments, the amount of a PPO-A protein is undetectable. In some embodiments, the amount of a PPO-B protein is 90% or less, 80% or less, 70% or less, 60% or less, 50% or less, 40% or less, 30% or less, 20% or less, 10% or less, or 5% or less of the amount of a PPO-B protein in a wildtype avocado plant cell, plant part, plant, or fruit. In some embodiments, the amount of a PPO-B protein is undetectable. In some embodiments, the amount of a PPO-C protein is 90% or less, 80% or less, 70% or less, 60% or less, 50% or less, 40% or less, 30% or less, 20% or less, 10% or less, or 5% or less of the amount of a PPO-C protein in a wildtype avocado plant cell, plant part, plant, or fruit. In some embodiments, the amount of any one or more of a PPO-D, PPO-E, PPO-F, PPO-G, and PPO-H protein is undetectable. In some embodiments, the amount of any one or more of a PPO-D, PPO-E, PPO-F, PPO-G, or PPO-H protein is 90% or less, 80% or less, 70% or less, 60% or less, 50% or less, 40% or less, 30% or less, 20% or less, 10% or less, or 5% or less of the amount of any one or more of a PPO-D, PPO-E, PPO-F, PPO-G, or PPO-H protein in a wildtype avocado plant cell, plant part, plant, or fruit. In some embodiments, the amount of any one or more of a PPO-D, PPO-E, PPO-F, PPO-G, and PPO-H protein is undetectable.

Introduction of Genome Editing Complexes into Plant Cells

Transient or stable insertion of recombinant DNA into the plant genome may be used to generate genome modifications using CRISPR or other forms of genome editing. In some embodiments, such genome modifications are achieved without inserting exogenous DNA into the plant cell. In some embodiments, a ribonucleotide particle or ribonucleoprotein (“RNP”) complex is preassembled and delivered to a target avocado plant cell. Since avocado is a clonally propagated woody perennial and exhibits outcrossing in nature, a mutation in both chromosomal alleles of one or more genes encoding traits of interest is advantageous. Use of ribonucleoprotein complexes (RNP) for genome editing can eliminate integration of nucleic acid into the plant genome and obviate the need for backcrossing and screening of progeny.

In some embodiments, the RNP complex is prepared using a molar ratio of genome editing nuclease to sgRNA of 1:10. In some embodiments, the molar ratio of genome editing nuclease to sgRNA ranges from 3:1, 1:1, 1:2, 1:3, or 1:6, as non-limiting examples.

In some embodiments, a plurality of RNP complexes is used to enable genome editing of multiple genes for traits of interest. In some embodiments, each RNP complex of the plurality of RNP complexes comprises a genome-editing nuclease and a gRNA sequence, where the plurality of RNP complexes comprise different gRNA sequences targeting at least two different genes. In some embodiments, the plurality of RNP complexes comprise different gRNA sequences targeting at least 3 or more different genes.

RNP complexes can be preassembled in vitro and introduced or delivered to an avocado plant cell. The methods of the present application are especially advantageous in crops such as avocado that are primarily clonally propagated since individual mutations are not able to be combined by traditional breeding methods. Instead, the methods of the present application allow the simultaneous editing of multiple different gene targets without the need to combine them by breeding.

In one embodiment, introduction of RNP complexes into plants may be performed by introducing the RNP complexes into protoplasts. Protoplasts may be made by any means known in the art such as, but not limited to, methods described in Engler & Grogan, “Isolation, Culture and Regeneration of Lettuce Leaf Mesophyll Protoplasts,” Plant Sci. Lett. 28:223-229 (1983); Nishio, “Simple and Efficient Protoplast Culture Procedure of Lettuce, Lactuca sativa L.,” Jap. J. Breeding 38(2):165-171 (1988), each of which is hereby incorporated by reference in its entirety. In some embodiments, equal ratio of each RNP complex is incubated with the protoplasts. In some embodiments, 1 nmol sgRNA is used per 10,000; 50,000; 100,000; 150,000; 200,000; 300,000 protoplasts; or any amount in between. In some embodiments, 1 nmol sgRNA is used per 200,000 protoplasts.

Methods of Generating Avocado Cells and Plants with PPO Mutations

Another aspect of the present application relates to a method of making an avocado plant cell comprising a loss of function mutation in polyphenol oxidase A (PPO-A). This method involves isolating nucellar tissue from an avocado plant, deriving a protoplast cell from the nucellar tissue, transfecting the protoplast cell with gene editing components, editing the protoplast cell genome to induce loss of function mutations in polyphenol oxidase A (PPO-A), and culturing the protoplast cell to make an avocado plant cell comprising a loss of function mutation in polyphenol oxidase A (PPO-A).

This aspect of the present application can be carried out with any of the embodiments disclosed herein.

In some embodiments, the methods of the present application involve isolating nucellar tissue from an avocado. In some embodiments, protoplast cells are derived from nucellar tissue. In some embodiments, the nucellar tissue is isolated from immature avocado fruits. In some embodiments, the immature avocado fruit is about 0.1 cm, 0.2 cm, 0.3 cm, 0.4 cm, 0.5 cm, 0.6 cm, 0.7 cm, 0.8 cm, 0.9 cm, 1.0 cm, 1.1 cm, 1.2 cm, 1.3 cm, 1.4 cm, 1.5 cm, or more than 1.5 cm in length. In some embodiments, the immature avocado fruit is about 0.2-1.0 cm in length. In some embodiments, pluripotent cells (“PC”) are derived from nucellar tissue. In some embodiments, protoplasts are derived from the nucellar tissue.

In some embodiments, protoplast cells are transfected with genome editing components. Plant protoplasts are enclosed only by a plasma membrane and will therefore take up macromolecules like RNP complexes. These protoplasts can be capable of regenerating whole plants. Transfection or transformation of protoplasts may be performed using any method known in the art including, but not limited to, polyethylene glycol treatment (Lelivelt et al., “Plastid Transformation in Lettuce (Lactuca sativa L.) by Polyethylene Glycol Treatment of Protoplasts,” Meth. Mol. Biol. 1132:317-330 (2014); Lelivelt et al., “Stable Plastid Transformation in Lettuce (Lactuca sativa L.),” Plant Mol. Biol. 58:763-774 (2005), each of which is hereby incorporated by reference in its entirety); using Sheen's protocol (Sheen, J. (2002) at URL genetics.mgh.harvard.edu/sheenweb/; Yoo & Sheen, “Arabidopsis Mesophyll Protoplasts: A Versatile Cell System for Transient Gene Expression Analysis,” Nat. Protocol. 2(7):1565-1572 (2007), each of which is hereby incorporated by reference in its entirety), microinjection, gene gun delivery (RNP biolistics or proteolistics), electroporation, gold nanoparticles, starch nanoparticles, silica nanoparticles, and the like.

In some embodiments, the protoplast cell genome is edited to induce loss of function mutations in polyphenol oxidase A (PPO-A) as described herein. In some embodiments, the protoplast cells are cultured to make an avocado cell with a loss of function mutation in polyphenol oxidase A (PPO-A). In some embodiments, the protoplast cell genome is further edited to induce loss of function mutations in polyphenol oxidase B (PPO-B). In other embodiments, the protoplast cell genome is further edited to induce loss of function mutations in polyphenol oxidase C (PPO-C). In some embodiments, the protoplast cell genome is further edited to induce loss of function mutations in additional avocado polyphenol oxidase genes as described herein.

In some embodiments, the genome edited protoplasts or plant cells of the present application may be regenerated and grown into plants. Methods of cultivating protoplasts into plants may be done by any means known in the art. See, for example, Witjaksono et al., Isolation, Culture and Regeneration of Avocado (Persea americana Mill.) protoplasts,” Plant Cell Reports 18:235-242 (1998), which is hereby incorporated by reference in its entirety. In some embodiments, the methods of the present application involve regenerating a plant from the plant cell having the genome edits in at least two different PPO genes. In other embodiments, a plant is regenerated from a plant cell having genome edits in at least 3, 4, 5, 6, 7, 8, 9, 10, or more than 10 different PPO genes.

The methods of the present application include formation of somatic embryos (“SE”) comprising genome edits. In some embodiments, proliferated calli from nucellar tissue are first subjected to a liquid pre-culture phase followed with a solid phase culture. In some embodiments, the liquid pre-culture calli are subjected to 2 weeks incubation in the dark in pre-culture media (see Shukla et al., “Nucellar Embryogenesis and Plantlet Regeneration in Monoembryonic and Polyembryonic Mango (Mangifera indica L.) Cultivars,” African Journal of Biotechnology 15:2814-2823 (2016), which is hereby incorporated by reference in its entirety). In some embodiments, the proliferated calli are placed under dark in pre-culture media for 1 week, 2 weeks, 3 weeks, 4 weeks, 5 weeks, 6 weeks, 7 weeks, or more than 7 weeks. In some embodiments, the calli are subcultured to fresh media. In some embodiments, the calli are then transferred to fresh SE maturation media in the dark at 25° C. for 4 weeks (see Shukla et al., “Nucellar Embryogenesis and Plantlet Regeneration in Monoembryonic and Polyembryonic Mango (Mangifera indica L.) Cultivars,” African Journal of Biotechnology 15:2814-2823 (2016), which is hereby incorporated by reference in its entirety). In some embodiments, the proliferated calli are placed under dark in SE maturation media for about 1 week, 2 weeks, 3 weeks, 4 weeks, 5 weeks, 6 weeks, 7 weeks, or more than 7 weeks in the dark. In some embodiments, the calli are transferred to fresh SE maturation media for an additional 3 weeks in the dark. In some embodiments, the proliferated calli are transferred to fresh SE maturation media and kept for about 1 week, 2 weeks, 3 weeks, 4 weeks, 5 weeks, 6 weeks, 7 weeks, or more than 7 weeks in the dark. In some embodiments, the proliferated calli are then cultured under the light for about 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks. In some embodiments, the proliferated calli are then cultured under the light for about 4 weeks to form SE. In some embodiments, the calli are sub-cultured to fresh media for about 1 week, 2 weeks, 3 weeks, 4 weeks, 5 weeks, 6 weeks, 7 weeks, 8 weeks, 9 weeks, 10 weeks, 11 weeks, 12 weeks, or more than 12 weeks to form SE.

An avocado cell, plant part, plant, or fruit comprising genome edits as described herein may be identified by comparing the sequence of the region of the gene targeted by the RNP complex with the sequence from a control plant. A control plant or plant cell may comprise a wildtype plant or cell, i.e., of the same genotype as the starting material for the genome editing. In some embodiments, DNA is extracted from an avocado cell, plant part, plant, or fruit, and the sequence around the target genome sites for the gRNA is evaluated. In some embodiments, Inference of CRISPR Edits (ICE) is used for analysis of genome edits (see Hsiau et al., “Inference of CRISPR Edits from Sanger Trace Data,” bioRxiv 251082 (2019), which is hereby incorporated by reference in its entirety). In some embodiments, the plant cell having genome edits is regenerated without the use of a selectable marker.

In some embodiments, the methods of the present application include elongating the shoot. In some embodiments, elongating the shoot includes incubating a plant part, e.g., isolated regenerable cell cluster, that has grown a shoot of at least 0.5 cm, e.g., at least 0.6 cm, at least 0.7 cm, at least 0.8 cm, at least 0.9 cm, at least 1 cm, at least 1.2 cm, at least 1.4 cm, at least 1.6 cm, at least 1.8, including at least 2 cm on elongation medium. In some embodiments, the plant part is incubated under light for 1-6 weeks, e.g., 1-4 weeks, 2-4 weeks, 3-4 weeks, 4-5 weeks, 5-6 weeks, or longer than 6 weeks on the shoot elongation medium.

In some embodiments, the methods of the present application include incubating a shoot on a suitable rooting medium. In some embodiments, vitrified shoot is incubated in the absence of any medium, e.g., in an empty petri dish, until vitrification is removed, before rooting.

In some embodiments, the method of mutating the PPO genes leaves no pest sequences in the genome of the avocado plant or plant cell. Through the use of RNP complexes for genome edits, the use of any exogenous DNA is avoided. In some embodiments, the genome editing components comprise ribonucleoprotein complexes (RNPs) without the use of plant pest sequences (such as Agrobacterium sequences, as one example). In some embodiments, the avocado plant, plant part, or fruit is free of exogenous DNA. In some embodiments, the avocado plant, plant part, or fruit is free of plant pest sequences.

A further aspect of the present application relates to a method of making an avocado plant cell comprising altered expression of a gene of choice. This method involves isolating nucellar tissue from an avocado plant, deriving a protoplast cell from the isolated nucellar tissue, transfecting the protoplast cell with gene editing components to edit the protoplast cell genome to alter the expression of a gene, and culturing the protoplast cell to make an avocado plant cell comprising altered expression of the gene.

This aspect of the present application can be carried out with any of the embodiments disclosed herein.

In some embodiments, the protoplast cell is transfected with gene editing components to edit the protoplast cell genome to alter the expression of a gene. In some embodiments, the protoplast cell is cultured to make an avocado cell with altered expression of the gene.

In some embodiments, this method allows altered expression of other genes in avocado through genomic editing of the plant genome to “knockout,” “knockin” or alter expression (increased or decreased) of genes related to a trait for which one desires an altered characteristic in the plant. The gene may be related to such traits as flavor, aroma, nutritional value, color, texture, allergen expression, pathogen resistance, abiotic stress tolerance, or any other desirable or useful trait.

Shelf Life of Avocados with Loss of Function PPO Mutations

In some embodiments, the avocado cell, plant, plant part, or fruit of the present application exhibits longer shelf life compared to a wildtype variety under the same conditions. Shelf life can be assessed by a number of factors including organoleptic scoring. For example and without limitation, organoleptic scores may be produced on a qualitative basis across several categories, including, for example and without limitation, color, off odor, aroma, moisture, texture, decay/mold, fruit discoloration, or taste. A total score combining values from each category provides an overall assessment of a plant, plant part, or fruit. In some embodiments, shelf life is scored in fresh avocado fruit by cutting the avocado fruit into pieces. In some embodiments, shelf life is scored after processing the avocado fruit. In some embodiments the fruit is cut and/or mashed, optionally mixed with other ingredients, and evaluated for organoleptic properties, especially color. In some embodiments, the shelf life is scored in an avocado plant, plant part, or fruit after storage under optimal conditions of light and temperature. In some embodiments, the shelf life is scored after storage under suboptimal conditions of light and temperature.

In some embodiments, the shelf life of the avocado fruit of the present application exhibits more than 1 hour, more than 2 hours, more than 3 hours, more than 4 hours, more than 5 hours, more than 6 hours, more than 7 hours, more than 8 hours, more than 9 hours, more than 10 hours, more than 11 hours, more than 12 hours, more than 13 hours, more than 14 hours, more than 15 hours, more than 16 hours, more than 17 hours, more than 18 hours, more than 19 hours, more than 20 hours, more than 21 hours, more than 22 hours, more than 23 hours, more than 24 hours, more than 25 hours, more than 26 hours, more than 27 hours, or more than 28 days of commercially-suitable shelf life, such as reduced browning, compared to a wildtype variety under the same conditions.

In some embodiments the harvested avocado fruit has reduced friction damage after transport. As used herein, “friction damage” is characterized by an oxidation of the tissue that later inclines downward and becomes necrotic. In some embodiments, the friction damage is reduced by 5%, 10%, 15%, 20%, 25%, 30%, 35%, 40%, 45%, 50%, 55%, 60%, 65%, 80%, 85%, 90%, 95%, or 100% compared to a wildtype avocado fruit after transport.

In some embodiments, the shelf life of the avocado cell, plant, plant part, or fruit of the present application exhibits reduced failure rate at days after harvest compared to a wildtype variety under the same conditions as assessed by organoleptic scoring. As used herein, the “failure rate” means the percentage of replicates at a particular time point of a shelf life study that, assessed by organoleptic scoring, is unsuitable for marketability.

In some embodiments, the shelf life of the avocado plant cell, plant, plant part, or fruit of the present application exhibits a reduced failure rate of 13 days after harvest as assessed by organoleptic scoring. In some embodiments, the avocado plant cell, plant, plant part, or fruit exhibits 0% failure rate 13 days post-harvest as assessed by organoleptic scoring. In some embodiments, the avocado plant cell, plant, plant part, or fruit exhibits less than less than 5%, less than 10%, less than 20%, less than 30%, less than 40%, or less than 50% failure rate 13 days post-harvest as assessed by organoleptic scoring. In some embodiments, the avocado plant cell, plant, plant part, or fruit exhibits less than 10% failure rate 13 days post-harvest as assessed by organoleptic scoring. In some embodiments, the shelf life of the avocado plant cell, plant, plant part, or fruit of the present application exhibits reduced failure rate of 21 days after harvest as assessed by organoleptic scoring. In some embodiments, the avocado plant cell, plant, plant part, or fruit exhibits 0% failure rate 21 days post-harvest as assessed by organoleptic scoring. In some embodiments, the avocado plant cell, plant, plant part, or fruit exhibits less than 5%, less than 10%, less than 20%, less than 30%, less than 40%, less than 50%, less than 60%, less than 70%, less than 80%, or less than 90% failure rate 21 days post-harvest as assessed by organoleptic scoring.

A recognized problem that is associated with harvested fruits is that the levels of plant phytochemicals, such as plant secondary metabolites, start to decrease almost immediately post-harvest. Such phytochemicals include vitamins, e.g., vitamins A, C, E, K, and/or folate, carotenoids such as beta-carotene, lycopene, the xanthophyll carotenoids such as lutein and zeaxanthin, phenolics comprising the flavonoids such as the flavonols (e.g., quercetin, rutin, caffeic acids), sugars, and other food products such as anthocyanins, among many others. In some embodiments the avocado cell, plant, plant part, or fruits of the present application also exhibit higher levels of plant phytochemicals compared to a wildtype variety.

In some embodiments, the avocado plant cell, plant, plant part, or fruits of the present application also exhibit higher levels of polyphenolics in comparison to a wildtype variety. In one embodiment, the level of polyphenolics is 5%, 10%, 15%, 20% or more than 20% higher than a wildtype variety. The avocado plant cell, plant, plant part, or fruits of the present application also retain higher levels of polyphenolics after harvest in comparison to a wildtype variety. In one embodiment, the level of polyphenolics is 5%, 10%, 15%, 20%, or greater than 20% higher than a wildtype variety at 7, 14, or 21 days after harvest.

One of skill in the art would readily understand that the methods described herein may be modified and optimized for particular embodiments of choice. The following examples are intended to illustrate but not limit the invention.

### EXAMPLES

### Example 1—Nucellar Callus Induction

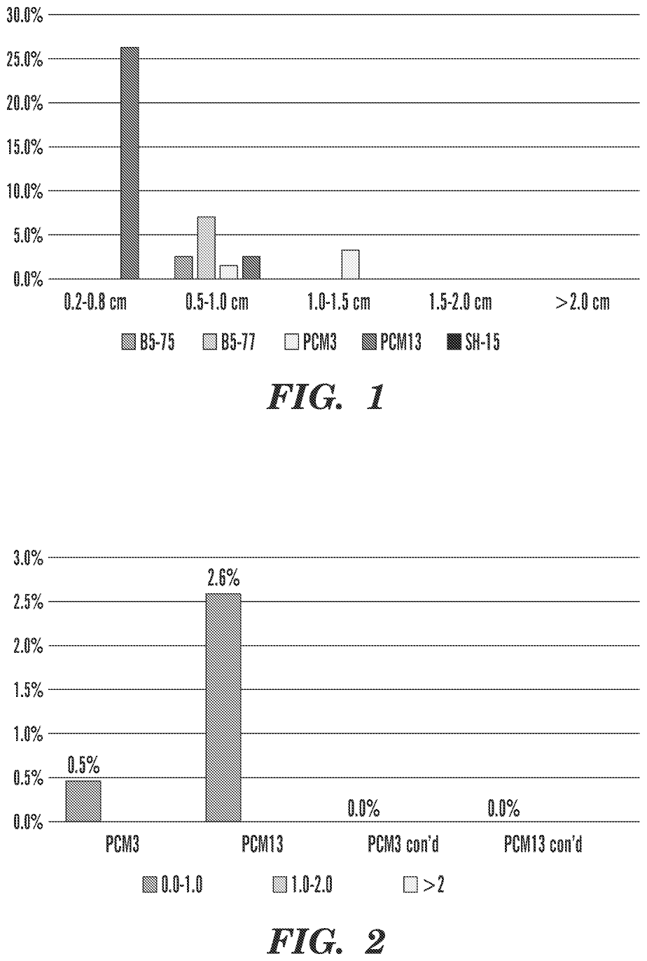

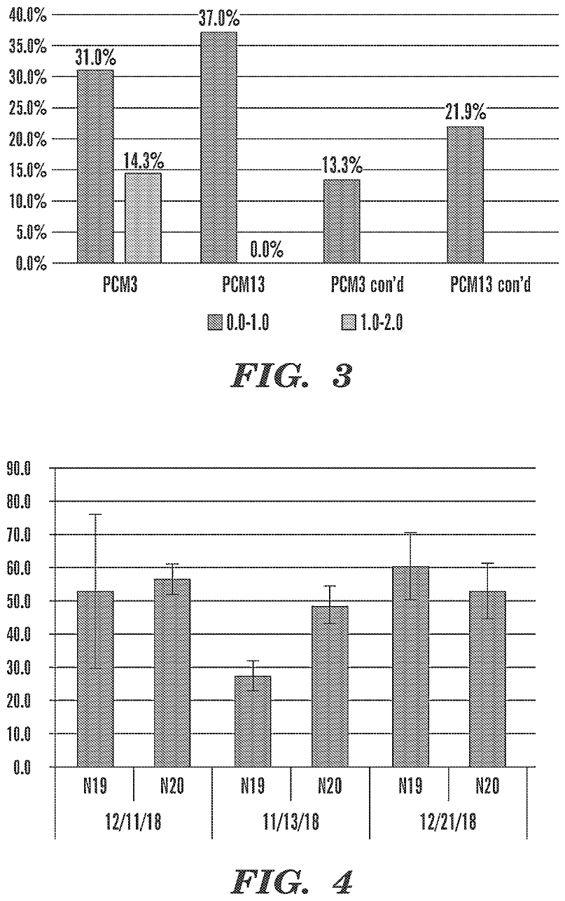

Immature avocado fruits are the only the source of nucellar (true-to-type) tissues. Experiments were conducted to optimize media and plant growth regulator (“PGR”) combinations for propagation of these tissues, and this involved testing fruits of different developmental sizes. The optimal fruit size to generate efficient callusing from nucellar tissue was obtained from fruits smaller than 1.0 cm in length. Callus induction and induction media were basically as described by Shukla et al., “Nucellar Embryogenesis and Plantlet Regeneration in Monoembryonic and Polyembryonic Mango (Mangifera indica L.) Cultivars,” African Journal of Biotechnology 15:2814-2823 (2016), which is hereby incorporated by reference in its entirety. The percentage of callus induction from nucellar tissue was 26.1% with fruit 0.2-0.8 cm using fruits obtained during the 2018 season (FIG. 1). In contrast, during 2019 the rate was 2.6% with fruits ˜1.0 cm as the fruits in 2019 were biased more toward 1.0 cm (FIG. 2). Although a reproducible method inducing true-to-type callus has been developed, the seasonal nature of the fruit availability makes it harder to keep a supply of young, regeneration competent callus. The fruit size <1.0 cm was also found suitable for inducing callus from zygotic tissues (FIG. 3).

Calli Proliferation/Maintenance

Maintenance of embryogenic calli was successfully obtained using a bi-weekly sub-culturing regiment as described in Shukla et al., “Nucellar Embryogenesis and Plantlet Regeneration in Monoembryonic and Polyembryonic Mango (Mangifera indica L.) cultivars,” African Journal of Biotechnology 15:2814-2823 (2016), which is hereby incorporated by reference in its entirety. Calli on the media are small early stage somatic embryos that do not mature past 1 mm length. The calli are also very friable and are suitable for liquid pre-culture treatment before somatic embryo maturation.

### Example 2—Somatic Embryo Maturation and Germination

Formation of somatic embryos (SE) is a key next step in the regeneration of avocado. A three-phase SE development protocol was optimized. Proliferated calli were first subjected to a liquid pre-culture phase followed with a solid phase culture. During the liquid pre-culture calli were subjected to 2 weeks incubation in the dark in pre-culture media (see Shukla et al., “Nucellar Embryogenesis and Plantlet Regeneration in Monoembryonic and Polyembryonic Mango (Mangifera indica L.) cultivars,” African Journal of Biotechnology 15:2814-2823 (2016), which is hereby incorporated by reference in its entirety). Then the calli were transferred to SE maturation media (Shukla et al., “Nucellar Embryogenesis and Plantlet Regeneration in Monoembryonic and Polyembryonic Mango (Mangifera indica L.) cultivars,” African Journal of Biotechnology 15:2814-2823 (2016), which is hereby incorporated by reference in its entirety) in the dark at 25° C. for 4 weeks. The calli were then transferred to fresh SE maturation media (Shukla et al., “Nucellar Embryogenesis and Plantlet Regeneration in Monoembryonic and Polyembryonic Mango (Mangifera indica L.) cultivars,” African Journal of Biotechnology 15:2814-2823 (2016), which is hereby incorporated by reference in its entirety) for an additional period of 3 weeks in the dark, followed by culturing under light (˜40 μmol m-2 s-1) at 25° C. for ˜4 weeks to initiate SE germination. Germination of SE is not a highly synchronous process. Therefore, additional sub-culturing of calli is often necessary until germination occurs.

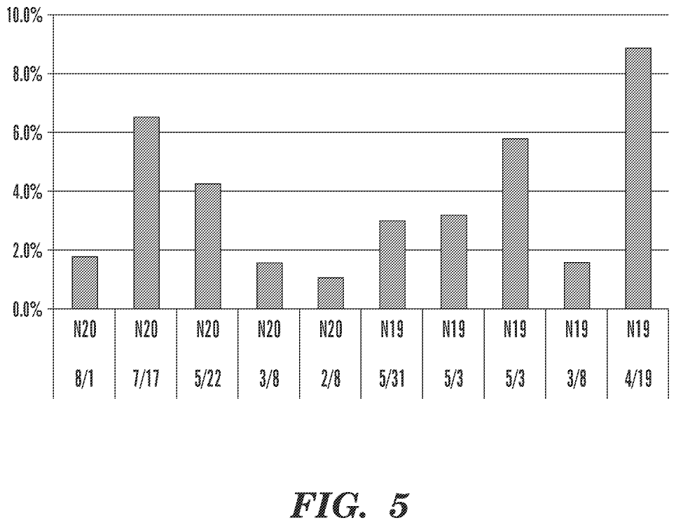

Initially, the somatic embryo (SE) regeneration method was able to generate only 3-4 SE per plate of ˜70 mg of small calli. Using the optimized method described above, 30-70 SE have been achieved per plate of ˜70 mg calli (FIG. 4), making it possible to generate 5000 SE per 1 gm of calli. It was observed that the SE regeneration rate was inversely proportional to the age of the calli line, making it important to induce a fresh culture of callus lines at least once every year. An average rate of 3.8% germination has been achieved (FIG. 5). The current system of regeneration is estimated to be capable of regenerating ˜200 germinated SE from 1 gram of starting callus.

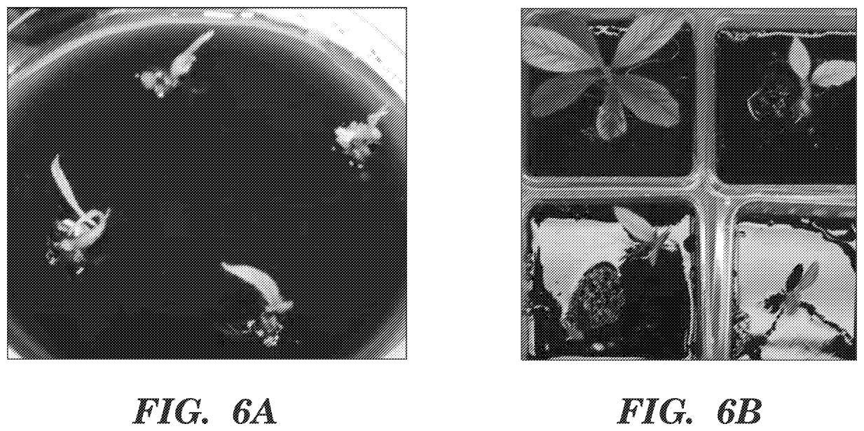

Shoot elongation of avocado plants was also achieved. Once shoots were rooted, they were tolerant of many different media. Production of a total of 149 shoots was achieved in initial experiments (see FIGS. 6A-B).

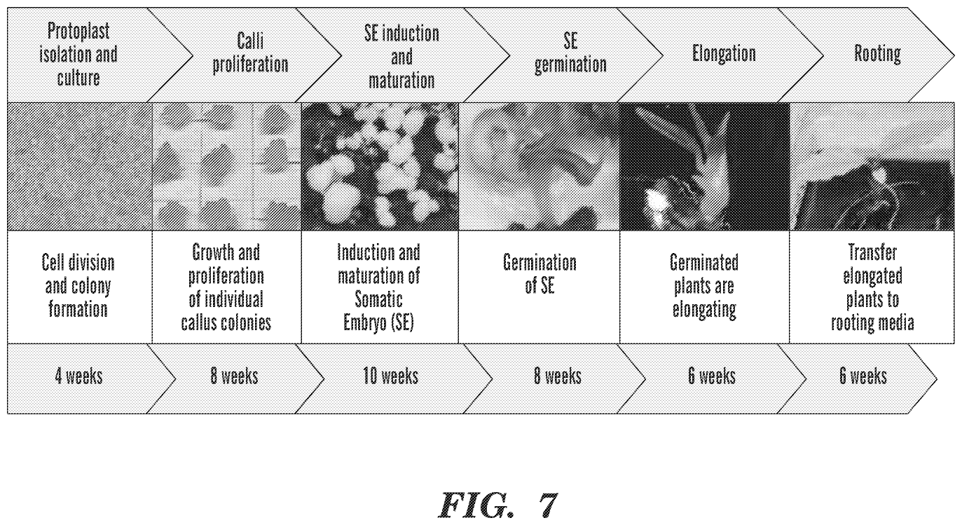

Suspension cell culture was established from the embryogenic calli described above to use as explant for protoplast isolation and regeneration following a published protocol (Witjaksono et al., “Isolation, Culture and Regeneration of Avocado (Persea americana Mill.) Protoplasts,” Plant Cell Reports. 18:235-242 (1998), which is hereby incorporated by reference in its entirety). The process and approximate timing of protoplast isolation and culture to rooting of plants in the procedure are shown in FIG. 7. Specifically, protoplasts were isolated and cultured for 4 weeks to allow cell division and colony formation. Next, growth and proliferation of individual callus colonies occurred during the next 8 weeks. After proliferation, the next 10 weeks involved the induction and maturation of somatic embryos. Germination of SE occurred over the next 8 weeks, followed by elongation of germinated plants over the next 6 weeks. After elongation, plants were transferred to rooting media for 6 weeks.

### Example 3—Genome Sequence of Avocado

Gene editing of PPO genes to create a non-browning trait in avocado requires knowledge of the avocado genome sequence. A limited amount of avocado genome sequence available publicly contained only 49% of the expected genes in avocado. An avocado genome sequencing project was implemented to generate a high-quality draft avocado DNA sequence rapidly and cheaply with “third-generation” sequencing technology. In brief, the avocado genomic sequences were assembled by using Verinomics (5 Science Park, New Haven, Conn.) pipeline. High molecular weight DNA was extracted and sequenced using Illumina NovaSeq 6000 S4 platform. The sequencing results were assembled and annotated by Verinomics and visualized through web-based JBrowse. The final assembly contains a 919 MB genome and is covered by 492 scaffolds.

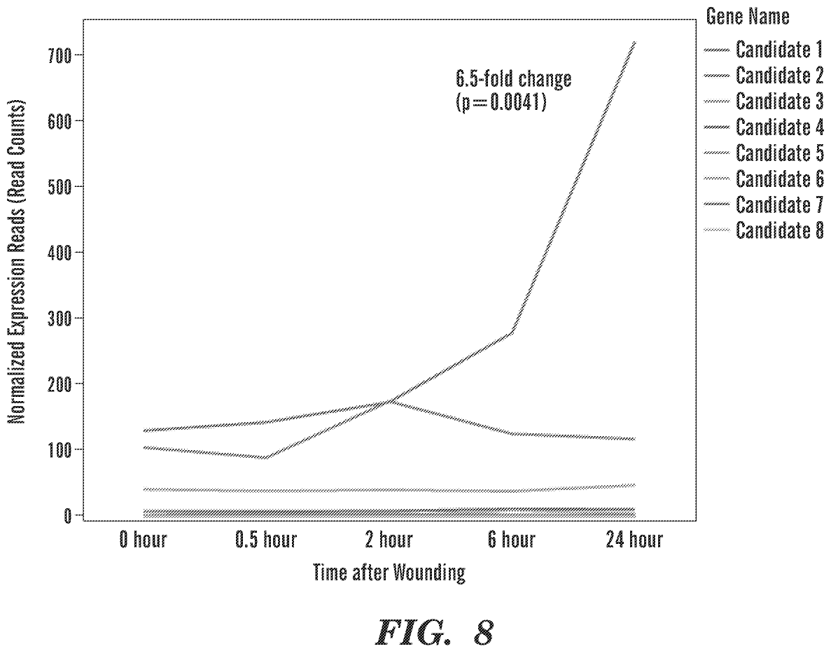

The DNA sequences of all PPO genes expressed in avocado fruit were obtained and was used to design highly specific CRISPR guide RNAs. Out of 8 PPO gene candidates (FIG. 8), the genomic location and structure of the two most highly expressed PPO genes in avocado fruit were identified. The sequences are provided in Table 1 below. The two most highly expressed PPO genes were named PPO-A (SEQ ID NO:1) and PPO-B (SEQ ID NO:4). The coding sequence of PPO-A is provided as SEQ ID NO:2, and the amino acid sequence of PPO-A is provided as SEQ ID NO:3. The coding sequence of PPO-B is provided as SEQ ID NO:5, and the deduced amino acid sequence of PPO-B is provided as SEQ ID NO:6. The genomic sequence, coding sequence, and amino acid sequence of PPO-C/PPO-C are SEQ ID NOs:7-9, respectively. The genomic sequence, coding sequence, and amino acid sequence of PPO-D/PPO-D are SEQ ID NOs:10-12, respectively. The genomic sequence, coding sequence, and amino acid sequence of PPO-E/PPO-E are SEQ ID NOs:13-15, respectively. The genomic sequence, coding sequence, and amino acid sequence of PPO-F/PPO-F are SEQ ID NOs:16-18, respectively. The genomic sequence, coding sequence, and amino acid sequence of PPO-G/PPO-G are SEQ ID NOs:19-21, respectively. The genomic sequence, coding sequence, and amino acid sequence of PPO-H/PPO-H are SEQ ID NOs:22-24, respectively.

The sequences of the avocado PPO-A-H genes, coding sequences, and amino acid sequences are provided in Table 1 (SEQ ID NOs:1-24). Exons in the genomic sequences are indicated in bold font.

TABLE 1
Exemplary Avocado PPO Sequences
Avocado PPO Sequences

PPO-A genomic sequence (5′ to 3′) (SEQ ID NO: 1)
ATGGCTTTCA CACTGAAAAG CACACCATCC CCGCTCTCTT CCTCCTCTTC AACCCCATTC
CATCAGCAAG CCAAGAAGAA ACCTCTTCTC CTTCCAAACC ATCGACCCCA CCACCTTCCT
CAACCAATCT CCTGCAATAG CAGCAACAAC AGTGAAAAAA ATGAAACCCA AAACCATGGT
AGAACTATCG ATAGAAGAAA TGTTCTTCTA GGCTTGGGAG GCCTCTATGG CGCTGCTACC
GCCTTCTCCA TCGACCCGAA GAAGGCCGCT GCGGCACCAG TTCTCCCACC TGATCTCTCC
CAATGTGGAG CAGCAGATCT CCCGGCTGGT GCCACCCCCA CCAACTGCTG CCCCCCCTTC
ACCCAAAAGA TTGTGGATTT CAAGCTCCCC TCCCCCTCCT CCCCCATGCG CGTTCGCCCT
GCCGCTCATC TTGCTGATAA AGAATACATA GCCAAGTATG AGAAGGCAAT TGCACTCATG
AAGGCTCTCC CAGCTGATGA CCCCAGGAAC TTCACTCAAC AGGTGAATAA ATTTTAAGCC
CTACATTTCA AAAAAAAAAA AAAAAATGGA CCCTATTCTG TCTTATTAAT TATAATAAGA
AGCATCACAT ACAATACATA ACAAAATTGA AGTCCTGCAT TTATGCAGCA GGAGGATTTT
ACTCTCCGCA ACTTTCATTT CAAAAGAGAG AGAGGAATAC TGCATTTCAA CTCTTAGTAT
ACGAAAGCAT GACCTTCTTT AGTCTCATTT TTTTTCTTAT GTTTTGCACC AAACGAACCA
AAACAGAGGT TGAATAGAAA ATGAGAACAA TTTTCATCTC GCATTAATCT AACTCAAATC
TATTCAAATT CTAATTCGAA CCTTTGATAT GGCAGGCCAA CGTCCACTGT GCCTACTGCG
ACGGCGCCTA CGACCAGGTT GGATTTCCTG ACTTGGAGCT CCAAGTCCAC AATGGGTGGC
TCTTCTTACC CTTCCATCGT TACTACCTCT ACTTCTATGA GAAGATCTTG GGCAAGTTGA
TTGGAGATGA GACATTTGCT CTCCCCTTCT GGAACTGGGA TGCACCGGGT GGAATGCCAA
TGCCGTCCAT GTACGCCAAA CCATCGTCGC CGCTCTACGA CGAGCTGAGA GACGCCAAGC
ACCAGCCGCC TACGCTGGTG GATCTGGACT ACAACTTCCA GGATCCCACC AACACCGACA
AGCAGCAGAT AGCCAGCAAC CTCTCCATCA TGTACCGGCA GGTGGTGTCG AATGGCAAGA
CGGCGCAGTT GTTCATGGGT GCGGCGTACC GGGCCGGCGG GGAGCCGGAC CCCGGTGCCG
GGTCGCTAGA GAACGTGCCG CATGGGCCGG TCCATATCTG GACCGGTGAC CGGACTCAGC
CCAACACGGA GAACATGGGG AACTTCTACT CGGCGGCAAG GGACCCGATC TTCTTCGCCC
ACCACTCGAA CGTCGACCGG ATGTGGAGCG TGTGGAAGAC CCTGGGAGGG AAGAGGAAGG
ACTTCACTGA CCCAGATTGG CTCAACTCGG GCTTCCTTTT CTATGATGAG AACAAGCAGC
TGGTCCGAGT CAAGGTCAAG GACTGCCTCG ACTCGGCTAA TCTTCGGTAA TTAAAGATCT
GTCTTTCTCA GTTTCTTATT CTAACTATAC TCTTCCAGAT GCCTGATCCC AACTCCTCTT
CCAAATTGGG TTTTAGGTCA GCAAATGGGA TAATGCTTTT AGTGTTAGTA ACCTAGTCTT
TAAGTTTCAA CTCTGAATTA ACTTGTGAGG TTCCTCAGTG GGGCTTACTT GAAAAATCTT
AAATCCTACC GGATGGATGT GCACGGGTTA CTCCAATAGA AGTTGACCTT ACCCTCAAAT
TGGTCACTGT AAAACATTTG AATATAGTGA TTAAGCCGAT GGTGGACCCA ACTCGTATTG
AAATTTGATG AGGACCAAAG CATTTATTTT GTGCCGTTTA AATATTCTTC CAAGTGATCC
AACTGAGGAG CCTTGTGAAT ATTGCACCAT GTCTGTCTGG CTTGAGAATC CTGACTTATA
AAATTCTCGG TCAACAAAAC TGAATAGGTG AACACAGCTG AATTACATTT TATGTTACAG
TTGAAAGTTT GATTTTACAA CGCTCCTATT TGATCTCACA TAAATATCTA AATTTTTCAA
AAATAAATAC CCTCTCTTGT ACAAATTTCA TTCAAATAAG AATTAATTAC ACATTCAGGT
TGATAAGTCA GAAAACATTT ATTGTAATCA ATTTGATTTT GACAAAGATA AAATCTTGGG
TTGGTTAGGT CTGAGTCAGT CAGTAAAGAT CCTTGGAAGA CTTTTCCTTG TACATTATTA
TTTCTTTGAA TTTGGCTTCC TAATCCCATT AATCTCCGGT CCACCAAACA CAGGTACACC
TACCAAGATG TTGAAATCCC ATGGCTCAAG TCCAGGCCAA CGCCTCTGAA GAAGAAGACC
GCCGCGAAGA AGGCCTTAAA AGGCAAGACC CCAACTGGGT TCCCTCGAGA CCTCGACACA
ATAGTGAAGG CTACGGTCAA GAGGCCAAAG AAGGGAAGGA GCAAGAAGCA GAAGGAGGAT
GAAGAAGAAG TGCTGGTGAT ACAAGGCATA GAGCTGGAGA GGGACGTCCG GGTGAAGTTC
GACGTGTTCT TAAACGTGGC CGAGGAAGAC GAGGGCTCGT GCGGTCCGAG CTCGACCGAG
TTTGTGGGGA GCTTCGTGAA CGTGCCCCAC AAGCATGGGA AGAAGACGAC CAAGTTGCAG
ACGTCCCTGA GGCTGGGGAT AACGGAGGTG TTGGAGGACC TGGAGGCTGA TGATGATGAT
GATGTGGTGG TGACTCTGGT CCCACGCCAA GGGAAGGATG TGGTGTCTGT TGGAGGGTTG
AAGATAGAAT TTAGTACCTG A
PPO-A coding sequence (5′ to 3′) (SEQ ID NO: 2)
ATGGCTTTCA CACTGAAAAG CACACCATCC CCGCTCTCTT CCTCCTCTTC AACCCCATTC
CATCAGCAAG CCAAGAAGAA ACCTCTTCTC CTTCCAAACC ATCGACCCCA CCACCTTCCT
CAACCAATCT CCTGCAATAG CAGCAACAAC AGTGAAAAAA ATGAAACCCA AAACCATGGT
AGAACTATCG ATAGAAGAAA TGTTCTTCTA GGCTTGGGAG GCCTCTATGG CGCTGCTACC
GCCTTCTCCA TCGACCCGAA GAAGGCCGCT GCGGCACCAG TTCTCCCACC TGATCTCTCC
CAATGTGGAG CAGCAGATCT CCCGGCTGGT GCCACCCCCA CCAACTGCTG CCCCCCCTTC
ACCCAAAAGA TTGTGGATTT CAAGCTCCCC TCCCCCTCCT CCCCCATGCG CGTTCGCCCT
GCCGCTCATC TTGCTGATAA AGAATACATA GCCAAGTATG AGAAGGCAAT TGCACTCATG
AAGGCTCTCC CAGCTGATGA CCCCAGGAAC TTCACTCAAC AGGCCAACGT CCACTGTGCC
TACTGCGACG GCGCCTACGA CCAGGTTGGA TTTCCTGACT TGGAGCTCCA AGTCCACAAT
GGGTGGCTCT TCTTACCCTT CCATCGTTAC TACCTCTACT TCTATGAGAA GATCTTGGGC
AAGTTGATTG GAGATGAGAC ATTTGCTCTC CCCTTCTGGA ACTGGGATGC ACCGGGTGGA
ATGCCAATGC CGTCCATGTA CGCCAAACCA TCGTCGCCGC TCTACGACGA GCTGAGAGAC
GCCAAGCACC AGCCGCCTAC GCTGGTGGAT CTGGACTACA ACTTCCAGGA TCCCACCAAC
ACCGACAAGC AGCAGATAGC CAGCAACCTC TCCATCATGT ACCGGCAGGT GGTGTCGAAT
GGCAAGACGG CGCAGTIGTT CATGGGTGCG GCGTACCGGG CCGGCGGGGA GCCGGACCCC
GGTGCCGGGT CGCTAGAGAA CGTGCCGCAT GGGCCGGTCC ATATCTGGAC CGGTGACCGG
ACTCAGCCCA ACACGGAGAA CATGGGGAAC TTCTACTCGG CGGCAAGGGA CCCGATCTTC
TTCGCCCACC ACTCGAACGT CGACCGGATG TGGAGCGTGT GGAAGACCCT GGGAGGGAAG
AGGAAGGACT TCACTGACCC AGATTGGCTC AACTCGGGCT TCCTTTTCTA TGATGAGAAC
AAGCAGCTGG TCCGAGTCAA GGTCAAGGAC TGCCTCGACT CGGCTAATCT TCGGTACACC
TACCAAGATG TTGAAATCCC ATGGCTCAAG TCCAGGCCAA CGCCTCTGAA GAAGAAGACC
GCCGCGAAGA AGGCCTTAAA AGGCAAGACC CCAACTGGGT TCCCTCGAGA CCTCGACACA
ATAGTGAAGG CTACGGTCAA GAGGCCAAAG AAGGGAAGGA GCAAGAAGCA GAAGGAGGAT
GAAGAAGAAG TGCTGGTGAT ACAAGGCATA GAGCTGGAGA GGGACGTCCG GGTGAAGTTC
GACGTGTTCT TAAACGTGGC CGAGGAAGAC GAGGGCTCGT GCGGTCCGAG CTCGACCGAG
TTTGTGGGGA GCTTCGTGAA CGTGCCCCAC AAGCATGGGA AGAAGACGAC CAAGTTGCAG
ACGTCCCTGA GGCTGGGGAT AACGGAGGTG TTGGAGGACC TGGAGGCTGA TGATGATGAT
GATGTGGTGG TGACTCTGGT CCCACGCCAA GGGAAGGATG TGGTGTCTGT TGGAGGGTTG
AAGATAGAAT TTAGTACCTG A
PPO-A amino acid sequence (SEQ ID NO: 3)
MAFTLKSTPS PLSSSSSTPF HQQAKKKPLL LPNHRPHHLP QPISCNSSNN SEKNETQNHG
RTIDRRNVLL GLGGLYGAAT AFSIDPKKAA AAPVLPPDLS QCGAADLPAG ATPTNCCPPF
TQKIVDFKLP SPSSPMRVRP AAHLADKEYI AKYEKAIALM KALPADDPRN FTQQANVHCA
YCDGAYDQVG FPDLELQVHN GWLFLPFHRY YLYFYEKILG KLIGDETFAL PFWNWDAPGG
MPMPSMYAKP SSPLYDELRD AKHQPPTLVD LDYNFQDPTN TDKQQIASNL SIMYRQVVSN
GKTAQLFMGA AYRAGGEPDP GAGSLENVPH GPVHIWTGDR TQPNTENMGN FYSAARDPIF
FAHHSNVDRM WSVWKTLGGK RKDFTDPDWL NSGFLFYDEN KQLVRVKVKD CLDSANLRYT
YQDVEIPWLK SRPTPLKKKT AAKKALKGKT PTGFPRDLDT IVKATVKRPK KGRSKKQKED
EEEVLVIQGI ELERDVRVKF DVFLNVAEED EGSCGPSSTE FVGSFVNVPH KHGKKTTKLQ
TSLRLGITEV LEDLEADDDD DVVVTLVPRQ GKDVVSVGGL KIEFST
PPO-B genomic sequence (5′ to 3′) (SEQ ID NO: 4)
ATGGCTATGG CATCCACATT TTTAAGCAAC AATAGCTTAG GGTCCGGTCT AAATACGAAG
GCCACCACCT CCTCTGCATG GCCTCTTCAC CAGCAAAGGA GTCAAGTTTC TGGTGGTGTA
CGTAGAAGGC ACAGCCGCCG TCAATCTCTT CTGATTTCAT GCAAAGGTGG ACATGATGCT
GATAATGCTG TCCCGTTTAT TGACCGTCGG AATATGCTTA TAGGCTTGGG AGGGCTGTAT
GGTGCAGCAA GTAGCATTGG TTTCGACGCC GTTGCCGCTC CGATTGCCCC ACCGGACTTA
TCCAAGTGCG GGCCGGCCGA TTTGCCGGCG GGTGCTATCC CAACAAACTG CTGCCCACCC
TTCAATGATA AGATTGTGGA CTTCAAGTTC CCATCTTTGA CCAAAATGAG GGTGCGGCCG
GCAGCTCACA GAGCGGCGGA CGACAAAGAG TACATGGAGA AGTTCACCAA GGCCGTAAAA
TTGATGAGGG AGCTTCCTAA GGACGACCCA AGGAACTTCA CGCAGCAGGC GAATGTGCAT
TGCGCCTACT GTGATGGTAC TGAGAAACTT TAAACATTAT CCACTTTTCA ATCAATTTAT
TTTTTCCGGA GATTTGACAC TTCAATGCCC TTTAAATATT TAATCTCCAA TTTTATTACT
AAGAATCTGT AAATGTCATC GGAGTCGTTT GACTCTGATA CACAATTTTT CTTTTTATTT
TTTACTTTTT TGCTCTCGAT CCCATGATAT CATGGGAGTT TAAAAACTCA TGATATAATG
CGATCCAAAT GTTAAAAGTT AAAAAGCTAC CTGTCATGGG ATTTAGAGCA AAAAGTAAAA
AAATAAAAAT ATACCAGAGT CAATCTGATG ACATTTAGAC ACTTTTAGTT ATGAAATTGG
AAATTTAGTG TTTAAGGGGC ATTGAAATGC CAAATCCTCT GTATTTATTG ATAGTTACTA
CAAGACTACA TACAATGAGA TGAATAAGAA AACTGGGTTT CATCTTAATA TCAATTTCAT
CCAATTATAA TAATGAGGGG TCATTTCTTA ATCTAATCTT GTTGCAATAA TATTAATGGT
CGCACTCTAA TATAACTACT AATCACAATT TTTCTAACCT GTATTCTCTA TAGGTGCATA
CGACCAGGTG GGCTTCCCTG ACCTGGAGTT GCAGGTGCAC AACTCATGGC TCTTTTTCCC
CTTCCACCGC TGCTACCTCT ACTTCTTCGA AAGGATCCTG GGCAAGCTGA TTGGGGATGA
GTCCTTCGCC ATCCCCTTCT GGAACTGGGA CGCCCCTAAA GGCATGATAA TGCCCCCCAT
ATACACGGAC CCATCATCGT CTCTCTACGA CAAGCTTCGC GATGCGGCCC ACCAGCCTCC
CAAGGTCATC GATCTCGACT ACAACGGCGT CGATCCCACC ACCACCGATC GTCAACAAAT
TATAGACAAT CTCACCATCA TGTACCGGCA AATGGTGTCC AACGCCAGGA CCCCTCAGCT
CTTCCTGGGC TCTCCATACC GGGCCGGGGA CAATCCTGAC CCAGGAGCCG GGTCGGTTGA
GAACGTTCCA CATGGGCCGG TCCATGTATG GACCGGGGAC CGGACACAGC CCAACGGTGA
GGACATGGGC AACTTCTACT CAGCTGCCCG CGACCCAATC TTCTATGCTC ACCATGCGAA
CGTGGACCGC ATGTGGACCC TGTGGAGGCA AATGGGGGGC ACACATAAGG ACTTCACGGA
CTCGGACTGG TTGGACGCTG GGTTCCTCTT TTATGATGAA AATGCCCAGC TGGTGAGAGT
GAAAGTTAGA GACTGCCTTG ACATTGCCAA GCTTGGATAC TCATACCAAC AAGTCGAGGT
CCCGTGGCTT AAGTCTCGCC CCACCACCAG ACGTGTGGCA GGTACCGCCT CGGTGGATTC
AGCCAAGAAG AAGGCGGATG CTACAGACGC AGAGGCCTAA TTCCCACGGA AGCAAGAAGG
TGTGTTGAAG GTGATCGTGA AGCATCCGTC AAAGTCAAGG AGCTCGACTC AGAAGGAGGA
AGAGGATGAG CTGCTTGTGA TAGACCAGAT TGAGGTGGGG CGTGATGTGC CTGCAAAGTT
CGATGTTTTC ATCAATGTGG AGGACCACAA GAAGCATGGG CCGGCCACGA GCGAGTTCGC
GGGCAGCTTT GTGAATGTGG CTCATAAGCA CAAGCATTCG AAGAAACCCA CGGTTCTCAA
GACGCGACTG AGGCTGGGGA TAACGGAGTT GCTGGAAGAC CTCGGAGCAG AGCAGGATGA
TGAAGTGGTG GTCACTTTGG TGCCGCGCTA TGGGAAGGAT GCAATCACTA TTGGAGAAGT
TCATATCGAA CACCATGCTG TTTCTTGA
PPO-B coding sequence (5′ to 3′) (SEQ ID NO: 5)
ATGGCTATGG CATCCACATT TTTAAGCAAC AATAGCTTAG GGTCCGGTCT AAATACGAAG
GCCACCACCT CCTCTGCATG GCCTCTTCAC CAGCAAAGGA GTCAAGTTTC TGGTGGTGTA
CGTAGAAGGC ACAGCCGCCG TCAATCTCTT CTGATTTCAT GCAAAGGTGG ACATGATGCT
GATAATGCTG TCCCGTTTAT TGACCGTCGG AATATGCTTA TAGGCTTGGG AGGGCTGTAT
GGTGCAGCAA GTAGCATTGG TTTCGACGCC GTTGCCGCTC CGATTGCCCC ACCGGACTTA
TCCAAGTGCG GGCCGGCCGA TTTGCCGGCG GGTGCTATCC CAACAAACTG CTGCCCACCC
TTCAATGATA AGATTGTGGA CTTCAAGTTC CCATCTTTGA CCAAAATGAG GGTGCGGCCG
GCAGCTCACA GAGCGGCGGA CGACAAAGAG TACATGGAGA AGTTCACCAA GGCCGTAAAA
TTGATGAGGG AGCTTCCTAA GGACGACCCA AGGAACTTCA CGCAGCAGGC GAATGTGCAT
TGCGCCTACT GTGATGGTAC TGAGAAACTT TAAACATTAT CCACTTTTCA ATCAATTTAT
TTTTTCCGGA GATTTGACAC TTCAATGCCC TTTAAATATT TAATCTCCAA TTTTATTACT
AAGAATCTGT AAATGTCATC GGAGTCGTTT GACTCTGATA CACAATTTTT CTTTTTATTT
TTTACTTTTT TGCTCTCGAT CCCATGATAT CATGGGAGTT TAAAAACTCA TGATATAATG
CGATCCAAAT GTTAAAAGTT AAAAAGCTAC CTGTCATGGG ATTTAGAGCA AAAAGTAAAA
AAATAAAAAT ATACCAGAGT CAATCTGATG ACATTTAGAC ACTTTTAGTT ATGAAATTGG
AAATTTAGTG TTTAAGGGGC ATTGAAATGC CAAATCCTCT GTATTTATTG ATAGTTACTA
CAAGACTACA TACAATGAGA TGAATAAGAA AACTGGGTTT CATCTTAATA TCAATTTCAT
CCAATTATAA TAATGAGGGG TCATTTCTTA ATCTAATCTT GTTGCAATAA TATTAATGGT
CGCACTCTAA TATAACTACT AATCACAATT TTTCTAACCT GTATTCTCTA TAGGTGCATA
CGACCAGGTG GGCTTCCCTG ACCTGGAGTT GCAGGTGCAC AACTCATGGC TCTTTTTCCC
CTTCCACCGC TGCTACCTCT ACTTCTTCGA AAGGATCCTG GGCAAGCTGA TTGGGGATGA
GTCCTTCGCC ATCCCCTTCT GGAACTGGGA CGCCCCTAAA GGCATGATAA TGCCCCCCAT
ATACACGGAC CCATCATCGT CTCTCTACGA CAAGCTTCGC GATGCGGCCC ACCAGCCTCC
CAAGGTCATC GATCTCGACT ACAACGGCGT CGATCCCACC ACCACCGATC GTCAACAAAT
TATAGACAAT CTCACCATCA TGTACCGGCA AATGGTGTCC AACGCCAGGA CCCCTCAGCT
CTTCCTGGGC TCTCCATACC GGGCCGGGGA CAATCCTGAC CCAGGAGCCG GGTCGGTTGA
GAACGTTCCA CATGGGCCGG TCCATGTATG GACCGGGGAC CGGACACAGC CCAACGGTGA
GGACATGGGC AACTTCTACT CAGCTGCCCG CGACCCAATC TTCTATGCTC ACCATGCGAA
CGTGGACCGC ATGTGGACCC TGTGGAGGCA AATGGGGGGC ACACATAAGG ACTTCACGGA
CTCGGACTGG TTGGACGCTG GGTTCCTCTT TTATGATGAA AATGCCCAGC TGGTGAGAGT
GAAAGTTAGA GACTGCCTTG ACATTGCCAA GCTTGGATAC TCATACCAAC AAGTCGAGGT
CCCGTGGCTT AAGTCTCGCC CCACCACCAG ACGTGTGGCA GGTACCGCCT CGGTGGATTC
AGCCAAGAAG AAGGCGGATG CTACAGACGC AGCATCCGTC TTCCCACGGA AGCTCGACTC
TGTGTTGAAG GTGATCGTGA AGAGGCCTAA AAAGTCAAGG AGCAAGAAGG AGAAGGAGGA
AGAGGATGAG CTGCTTGTGA TAGACCAGAT TGAGGTGGGG CGTGATGTGC CTGCAAAGTT
CGATGTTTTC ATCAATGTGG AGGACCACAA GAAGCATGGG CCGGCCACGA GCGAGTTCGC
GGGCAGCTTT GTGAATGTGG CTCATAAGCA CAAGCATTCG AAGAAACCCA CGGTTCTCAA
GACGCGACTG AGGCTGGGGA TAACGGAGTT GCTGGAAGAC CTCGGAGCAG AGCAGGATGA
TGAAGTGGTG GTCACTTTGG TGCCGCGCTA TGGGAAGGAT GCAATCACTA TTGGAGAAGT
TCATATCGAA CACCATGCTG TTTCTTGA
PPO-B amino acid sequence (SEQ ID NO: 6)
MAMASTFLSN NSLGSGLNTK ATTSSAWPLH QQRSQVSGGV RGRHSRRQSL LISCKGGHDA
DNAVPFIDRR NMLIGLGGLY GAASSIGFDA VAAPIAPPDL SKCGPADLPA GAIPTNCCPP
FNDKIVDFKF PSLTKMRVRP AAHRAADDKE YMEKFTKAVK LMRELPKDDP RNFTQQANVH
CAYCDGAYDQ VGFPDLELQV HNSWLFFPFH RCYLYFFERI LGKLIGDESF AIPFWNWDAP
KGMIMPPIYT DPSSSLYDKL RDAAHQPPKV IDLDYNGVDP TTTDRQQIID NLTIMYRQMV
SNARTPQLFL GSPYRAGDNP DPGAGSVENV PHGPVHVWTG DRTQPNGEDM GNFYSAARDP
IFYAHHANVD RMWTLWRQMG GTHKDFTDSD WLDAGFLFYD ENAQLVRVKV RDCLDIAKLG
YSYQQVEVPW LKSRPTTRRV AGTASVDSAK KKADATDAAS VFPRKLDSVL KVIVKRPKKS
RSKKEKEEED ELLVIDQIEV GRDVPAKFDV FINVEDHKKH GPATSEFAGS FVNVAHKHKH
SKKPTVLKTR LRLGITELLE DLGAEQDDEV VVTLVPRYGK DAITIGEVHI EHHAVS
PPO-C genomic sequence (5′ to 3′) (SEQ ID NO: 7)
ATGGAAGCAA AGCATTGGTT CTCTGTAGTA CTGCTGACTC TCCTTCTAGT TGGGCTGTCA
ATAAATCTTC TCCATGATTC AAACTCTTCT TTGAGGTATC GAAGTTTTTT CTATATTTGA
TTCTTTTAGA TCTTGTTTTT GGCTTTTGTC TTTTTTTTTT TTTTTTTTTT TTTTTTTTTG
GAAGTTGGGG TACAAGTGAT CTGTTTATAG AAATATGAAA AGGGTTTTTA TATAACTTGC
ATAGATTTTT GATGGATACA ATCAAACACA AATTGAAGTT TTCTAAATTG GTTTTTTGTA
ACAATTGTGT AGGGATCTAA GGGGATTGAA TGAGAAAAAC CCAGCGGCCT ACATCTCGAC
ATCATTTCAA TTGATCCAAA GCATGATCCC TTCAATCTGG GAAGGTCGGT CTTCAGATCC
TGAAGTAGCA AAGCAAACAG GTGGGAGGCC AATAGCTCCA AACCTTGCCA CATGCCACAA
ATCGCTCTCC GATGCGGGTC GTCCAGTCTT TTGCTGCCCA CCCAAACGCG AATCCGAAGA
GTCCGTCATC GACTTCAAAT TCCCAAGCCC TTCCACACCC AAACGGATCC GCCGACCCGC
CCACCTCGTA GACGACGACT ACCTCGCCAA GTACCAGAGA GGCGTGACCT TGATGAAGCA
ACTCGACACC AGCGACCCTC GCAACTTCAT GCGCCAGGCC AACATCCACT GCATCTTCTG
CACTGGAGCC TACACCCAAG TCAACTCCTC CCACCTCCTC AACATCCATA GATCATGGTT
CTTCTTCCCA TGGAGATGAC TGATGATCTA CTTCCATGAG AGGATCCTTG GAAAGCTGAT
TGGCACCGTT ACCTTCGCGC TCCCCTACTG GAACTGGGAC AACCCACCTG GCATGATCAT
CCCTCACTAC TACATGAATG GGTCTTTCGT CGACAAGGAT CGGGACCACG CCCATCTCCC
ACCCCAGGTT GCAGACATCA GCTTCGACTA CGTTGAGAGC GGACTCGGCC CTGAGGAGCA
GATAGAATCG AACCTCCACT TCATGTACCA TCAGATGGTG TCTGGTGCGA AGAAGGTCGA
GCTCTTCATG GGCTGCAAGC GAACCGCTGG GGAAGAGGGC GAGTGCGATG GTCCCGGCAC
GGTCGAGGTC GCACCCCACA ACGCTCTCCA CACGTGGGTG GGAAGCAATC TCCAGCCCGA
GAGGGAGAAC ATGGGTGCCT TCTACTCGGC TGCTCGTGAC CCCGTTTTCT ACGCCCACCA
TGCCAACATT GACCGGCTCT GGACGGTTTG GAGAAAGCTT AGGGGCAACG TGCCCGAGAT
TGTGGACCCG GCTTGGCTCG ACTCCTACTT TTACTTCCAC GACGAGAACG CTCAGCTCGT
TCGGATCAAG ATCCGAGATG CTCTTGACAT GGACAGGCTC GGTTATGGCT ACGAAGATAT
TGACCTCCCA TGGCTAAATG CCAGGCCCAA ACCCTCCGTC CCACCCAAAA TTGCGAAGGC
AGTGTTGAAG TTGAGAGAAC TAAACCAGAA CGGATTGCAG TCCCCAGCCC TCTTTAGCCC
TGACTTCGGA CCCGAGGGTC GGATCCTTGA CAGCACCATA AGAGCCAAGG TCCAGAGGCC
AAAGAGGTAC AGAAGCAAGA AGGAAAAAGA GGAAGAAGAG GAGGTTTTGG TTGTTTATGG
TATTGATATT AAAAGAGATA TGTATGTGAA GTTCGATGTC TACGTGAACG TGGTTGATGA
AAAGAATACG GGTCCTGAGG GTAGAGAGTT TGCGGGCACC TTCGTTAACG TGCGCCATGG
CGTGACAACA GTGTTGAACG AGGGTGATTC GAAGATGAAG ATGAAGAGCA CGCTCAAGTT
GGGGATTTCG GAGCTGTTGG AGGATTTGGA AGCTGATGAG GATGAGAGCG TTTGGGTTAC
ATTGTTGCCT AGAGGAGGGA CTGGTGTCAA TACTACTGTT GATGGAATAA GGATTGAGTA
CATGCGATGA
PPO-C coding sequence (5′ to 3′) (SEQ ID NO: 8)
ATGGAAGCAA AGCATTGGTT CTCTGTAGTA CTGCTGACTC TCCTTCTAGT TGGGCTGTCA
ATAAATCTTC TCCATGATTC AAACTCTTCT TTGAGGGATC TAAGGGGATT GAATGAGAAA
AACCCAGCGG CCTACATCTC GACATCATTT CAATTGATCC AAAGCATGAT CCCTTCAATC
TGGGAAGGTC GGTCTTCAGA TCCTGAAGTA GCAAAGCAAA CAGGTGGGAG GCCAATAGCT
CCAAACCTTG CCACATGCCA CAAATCGCTC TCCGATGCGG GTCGTCCAGT CTTTTGCTGC
CCACCCAAAC GCGAATCCGA AGAGTCCGTC ATCGACTTCA AATTCCCAAG CCCTTCCACA
CCCAAACGGA TCCGCCGACC CGCCCACCTC GTAGACGACG ACTACCTCGC CAAGTACCAG
AGAGGCGTGA CCTTGATGAA GCAACTCGAC ACCAGCGACC CTCGCAACTT CATGCGCCAG
GCCAACATCC ACTGCATCTT CTGCACTGGA GCCTACACCC AAGTCAACTC CTCCCACCTC
CTCAACATCC ATAGATCATG GTTCTTCTTC CCATGGCACC GTTTGATGAT CTACTTCCAT
GAGAGGATCC TTGGAAAGCT GATTGGAGAT GACACCTTCG CGCTCCCCTA CTGGAACTGG
GACAACCCAC CTGGCATGAT CATCCCTCAC TACTACATGA ATGGGTCTTT CGTCGACAAG
GATCGGGACC ACGCCCATCT CCCACCCCAG GTTGCAGACA TCAGCTTCGA CTACGTTGAG
AGCGGACTCG GCCCTGAGGA GCAGATAGAA TCGAACCTCC ACTTCATGTA CCATCAGATG
GTGTCTGGTG CGAAGAAGGT CGAGCTCTTC ATGGGCTGCA AGCGAACCGC TGGGGAAGAG
GGCGAGTGCG ATGGTCCCGG CACGGTCGAG GTCGCACCCC ACAACGCTCT CCACACGTGG
GTGGGAAGCA ATCTCCAGCC CGAGAGGGAG AACATGGGTG CCTTCTACTC GGCTGCTCGT
GACCCCGTTT TCTACGCCCA CCATGCCAAC ATTGACCGGC TCTGGACGGT TTGGAGAAAG
CTTAGGGGCA ACGTGCCCGA GATTGTGGAC CCGGCTTGGC TCGACTCCTA CTTTTACTTC
CACGACGAGA ACGCTCAGCT CGTTCGGATC AAGATCCGAG ATGCTCTTGA CATGGACAGG
CTCGGTTATG GCTACGAAGA TATTGACCTC CCATGGCTAA ATGCCAGGCC CAAACCCTCC
GTCCCACCCA AAATTGCGAA GGCAGTGTTG AAGTTGAGAG AACTAAACCA GAACGGATTG
CAGTCCCCAG CCCTCTTTAG CCCTGACTTC GGACCCGAGG GTCGGATCCT TGACAGCACC
ATAAGAGCCA AGGTCCAGAG GCCAAAGAGG TACAGAAGCA AGAAGGAAAA AGAGGAAGAA
GAGGAGGTTT TGGTTGTTTA TGGTATTGAT ATTAAAAGAG ATATGTATGT GAAGTTCGAT
GTCTACGTGA ACGTGGTTGA TGAAAAGAAT ACGGGTCCTG AGGGTAGAGA GTTTGCGGGC
ACCTTCGTTA ACGTGCGCCA TGGCGTGACA ACAGTGTTGA ACGAGGGTGA TTCGAAGATG
AAGATGAAGA GCACGCTCAA GTTGGGGATT TCGGAGCTGT TGGAGGATTT GGAAGCTGAT
GAGGATGAGA GCGTTTGGGT TACATTGTTG CCTAGAGGAG GGACTGGTGT CAATACTACT
GTTGATGGAA TAAGGATTGA GTACATGCGA TGA
PPO-C amino acid sequence (SEQ ID NO: 9)
MEAKHWFSVV LLTLLLVGLS INLLHDSNSS LRDLRGLNEK NPAAYISTSF QLIQSMIPSI
WEGRSSDPEV AKQTGGRPIA PNLATCHKSL SDAGRPVFCC PPKRESEESV IDFKFPSPST
PKRIRRPAHL VDDDYLAKYQ RGVTLMKQLD TSDPRNFMRQ ANIHCIFCTG AYTQVNSSHL
LNIHRSWFFF PWHRLMIYFH ERILGKLIGD DTFALPYWNW DNPPGMIIPH YYMNGSFVDK
DRDHAHLPPQ VADISFDYVE SGLGPEEQIE SNLHFMYHQM VSGAKKVELF MGCKRTAGEE
GECDGPGTVE VAPHNALHTW VGSNLQPERE NMGAFYSAAR DPVFYAHHAN IDRLWTVWRK
LRGNVPEIVD PAWLDSYFYF HDENAQLVRI KIRDALDMDR LGYGYEDIDL PWLNARPKPS
VPPKIAKAVL KLRELNQNGL QSPALFSPDF GPEGRILDST IRAKVQRPKR YRSKKEKEEE
EEVLVVYGID IKRDMYVKFD VYVNVVDEKN TGPEGREFAG TFVNVRHGVT TVLNEGDSKM
KMKSTLKLGI SELLEDLEAD EDESVWVTLL PRGGTGVNTT VDGIRIEYMR
PPO-D genomic sequence (5′ to 3′) (SEQ ID NO: 10)
ATGGAAAGTG GCGTCTGCTA CAGAGGAGGA ATTCCTGCCT TTGAAGCTTT GCCCTCTGGG
GTGAAGTCCA AAGGAGTTCT TACTGTTTCC GCCTCAGCCA TCCGATCTCG CTACGTTGCT
AGTATCTCCA TCGTTCGTTC TCAGGTTTGA GCTTCTGATC TGTGTAATCT AGTCTCTTTT
TAATAAAATC CATTTGAATG TTTTGTTGGG TTTTTAGTGA TTGTTAGGAT CTGGTGAGTG
ATGGGGTGGG ATTTGTTGTT TGTATTTCTT TTGATCGAGT TACGAGAAAT AGAACCGTTT
GTTGATAAAC AATGCAATTT TAAGATCTTC AACATGGGGT TCGGCTGGAA TTGGAAGATG
AAGACAGATG TTATAACCAA CGGTCTTGAT TTAGTAGTAG TAGGACACAC AAGTCTGTTT
CTGGGGGTGT TTGATTCGTG TCTTTTTTGG GCTTTGGGCC GTCTATATTG GGATCCAAGA
TATAAAGGGA CCAGATCGAT TGGTGGGGTT GCCCAGTGTT TTGTGGGAGC ACATGGTAGA
TTGTGCGATG GCACTGAAAG CTTGAGACAC AACCACTCTA GTGATGTTGA TGTTCCTTGA
ATGGAAAAGA TGAGACATTT CTAATGTTTT TATTTCTATT TCAAAGCCAT GAAATAGAAG
AAATTTATGT TCTTTTTTAA GTTGGAATAG ACCCCCACAA CCCCTGCATA GCAGGATGAT
GATAACAAAG CCATCTTCCT TTGGGCACAC TTAGTGGGCC CCACAAACTC TCCCATTTTC
ATAGAATTGG TTGTGGGTTC CTGCTTCTAA ATCTGTAGAG AGGATCCCTG TCCTTTTCTA
AAAGATCCTG AAATGAATGT ACAATTTTTT TTTTTGACAC ATTGGTTGTC TAGTATTGGT
GGCTTTTGAT CCCAAAGGAG AAACATTTCA CCATCGTCTT CATTTTAGTC TTATCCTATG
AAGGGGAGTC TTATTTTAGT CTTTAATTTG CCTTTTTTTC ATAATGTAGG GTATGGTCAA
CTTCTGTGGA AGCCGGCAAT TTGGAGAGAA GGTTAAGTGT GGCACTCTGA GAAGCCCGGC
TACATTTGTC ACAGTCGCCA GTGCAGAGTC TAGTAAGTGT GATTTTAGGA GTGTGGCGAC
ACCCCTCGAA CCACAATCAT CGGCTGGAAA GTTTTTGAGT GATATATTGA AGAATCACCC
TCACATTTTC CATGTGGCTG CTGCGGAGCA GCTGGAGCAT TTGGCTGCAG ATAGGGATGA
TGCTGTCGCT CGGCGGGAGC AGAGCTTGGG TTCACCTGAA TCATGCCTTC ATAGGTTAGT
ATTATTAACT CTTGATTTCT TCAATCATCA AATGTGGATT TAAACTGACA AATGTTCTTT
TTGGTTCCCG TGATCAACTG CTCTTTCTCG TTTGATCATG ATTTTCTGTC ACTGGTAGTA
TTGCCTATTA AAGGGTGCAA TTTTTTAAAC ATGTTATGTT CATTGTTTCA ATTGACCGGA
CTGCAGTGTC TAAATAGTTT TTCATCTGCT GGTCCATCAT CTTCTGTTAC AATTATTAAG
ATGTGCATTA CATCCTATAT TTATGCTTGC ATGTATAGAC CTGTGTTACC TATAACTGTT
GAAGTATGAA AAATGTTAGT GCCAAATGCA GTAGACACAT TGCTCAAGAT ATGTGAAAGC
ATAACAACTA TTAATGATTT CAGTCACTAT TTGATATGGT ATGTTTTGCC AATTTCTACG
ATTTCCAACC TAGATCTTGG GCAATCACCA AGATTGGAAC ACCCTAGGCA GCATCTCAAC
CATATTTTAT GGATTGTACA CATTGTTGCA GAAGTTACAA ATTAAAGCTC TTGAAATGAT
TGTGTTGTTT TGGATGCGTG ATTAAGGAAT GGTATGATTC ACATAACCAA TTCTCATGCA
AGAGTGTGTG TGCATGTGGA ACATATGGCT GGTGGTTCAA ACCATAGGTT CTGTTGGGCT
TGAGTTCCTT CAGGCACTGA ATTTTCAATT GGTCAGGCGA GGCCTATTCA AATTGACAGT
TCTAGAGCCT CGTCCATTGA CAGCCCTACC CAGTATAAAT AATCTTTAAC TCAAAATTGC
CTAGATCGGA CAATTGTATT TCACGTGTGT CACCAGTTAC CCTGTTAACA AATGTGTGAT
ATTGGATGCC AGGAGAATTG CAGAAATGAA GGAGGGTGAG TGCCAAATTG CGATCGAAGA
GGTCATGTAC ATGCTAGTTG TTCGAAAGTT CTCTGAGATT GATGTCCCAA TGGTTCCAAG
ATTATCCAAA TGCATCAACA ATGGGAGACT AGATATATGG ACGACCAAGG ACAGAGAACT
AGAGTCCATT CATAGCTTAG ATGTTCTGGA ATTGATTAGG GAACATCTCT CAACCATTCT
AGGCTGGAGA GGAAAATCCG ATGTCACAGA TAACTGGACA ACAACTCAGA TTTGCAGGCT
GCAGCTTGGC CGAATTTATG CTGCTTCCAT CATGTATGGG TACTTTCTGA AATCTGCCTG
CCTGCGGCAC CGCCTGGAGC TAAATCTCAG TCTGACTCAT GTAGACCTCC CTCCTGGACA
TGAGATTGAA CACCCACTTG CAGAAAGGCG GCCTTGTTCA CTTGGAAATC TTGCTGTTGC
TGGCTGTCCA AATGATACAA TATCTTCATT GTATCAAGGA TCAGGAAGGG ACAGGAGAAC
TGAGAAGCTG AAGAGGTATT TGATGGGATT TGATCCCGAG ACTTTACAGA GATGTGCAAA
GTTGAAATCG CAGGAAGCAG TAAACCTTAT TGAGAAGCAC AGTTGGGCTC TGTTTGGAGA
GGACAATGAG TCAGGTTCTA TAGACAGCGA CGAGGCGATT GCTGTCACAT TTTCAAGCCT
GAAGAGGTTG GTTTTGGAGG CTGTTGCATT TGGGTCTTTC CTTTGGGATG TGGAAAGGTA
TGTTGGTTCC TTATACAGGT TAAAGATGAC CTAA
PPO-D coding sequence (5′ to 3′) (SEQ ID NO: 11)
ATGGAAAGTG GCGTCTGCTA CAGAGGAGGA ATTCCTGCCT TTGAAGCTTT GCCCTCTGGG
GTGAAGTCCA AAGGAGTTCT TACTGTTTCC GCCTCAGCCA TCCGATCTCG CTACGTTGCT
AGTATCTCCA TCGTTCGTTC TCAGGGTATG GTCAACTTCT GTGGAAGCCG GCAATTTGGA
GAGAAGGTTA AGTGTGGCAC TCTGAGAAGC CCGGCTACAT TTGTCACAGT CGCCAGTGCA
GAGTCTAGTA AGTGTGATTT TAGGAGTGTG GCGACACCCC TCGAACCACA ATCATCGGCT
GGAAAGTTTT TGAGTGATAT ATTGAAGAAT CACCCTCACA TTTTCCATGT GGCTGCTGCG
GAGCAGCIGG AGCATTTGGC TGCAGATAGG GATGATGCTG TCGCTCGGCG GGAGCAGAGC
TTGGGTTCAC CTGAATCATG CCTTCATAGG AGAATTGCAG AAATGAAGGA GGGTGAGTGC
CAAATTGCGA TCGAAGAGGT CATGTACATG CTAGTTGTTC GAAAGTTCTC TGAGATTGAT
GTCCCAATGG TTCCAAGATT ATCCAAATGC ATCAACAATG GGAGACTAGA TATATGGACG
ACCAAGGACA GAGAACTAGA GTCCATTCAT AGCTTAGATG TTCTGGAATT GATTAGGGAA
CATCTCTCAA CCATTCTAGG CTGGAGAGGA AAATCCGATG TCACAGATAA CTGGACAACA
ACTCAGATTT GCAGGCTGCA GCTTGGCCGA ATTTATGCTG CTTCCATCAT GTATGGGTAC
TTTCTGAAAT CTGCCTGCCT GCGGCACCGC CTGGAGCTAA ATCTCAGTCT GACTCATGTA
GACCTCCCTC CTGGACATGA GATTGAACAC CCACTTGCAG AAAGGCGGCC TTGTTCACTT
GGAAATCTTG CTGTTGCTGG CTGTCCAAAT GATACAATAT CTTCATTGTA TCAAGGATCA
GGAAGGGACA GGAGAACTGA GAAGCTGAAG AGGTATTTGA TGGGATTTGA TCCCGAGACT
TTACAGAGAT GTGCAAAGTT GAAATCGCAG GAGGTTGGTT ACCTTATTGA TTGCATTTGG
TGGGCTCTGT TTGGAGAGGA GAAGCAGTAA TTGGAGGCTG GAAGCACAGT GTCTTTCCTT
GTCACATTTT CAAGCCTGAA CAATGAGTCA TGGTTCCTTA ACAGCGACGA AGATGACCTA
TGGGATGTGG AAAGGTATGT GGTTCTATAG TACAGGTTAA GGCGATTGCT A
PPO-D amino acid sequence (SEQ ID NO: 12)
MESGVCYRGG IPAFEALPSG VKSKGVLTVS ESSKCDFRSV SISIVRSQGM GKFLSDILKN
EKVKCGTLRS PATFVTVASA ASAIRSRYVA ATPLEPQSSA VNFCGSRQFG HPHIFHVAAA
EQLEHLAADR DDAVARREQS LGSPESCLHR RIAEMKEGEC QIAIEEVMYM LVVRKFSEID
VPMVPRLSKC INNGRLDIWT TKDRELESIH LELNLSLTHV HLSTILGWRG PLAERRPCSL
TQICRLQLGR IYAASIMYGY SLDVLELIRE GRDRRTEKLK KSDVTDNWTT LQRCAKLKSQ
GNLAVAGCPN DTISSLYQGS FLKSACLRHR RYLMGFDPET DLPPGHEIEH EAVNLIEKHS
WALFGEDNES GSIDSDEAIA VTFSSLKRLV LEAVAFGSFL WDVERYVGSL YRLKMT
PPO-E partial genomic sequence (5′ to 3′) (SEQ ID NO: 13)
ATGGAAAGTG GCGTTTGCTG TGGTAGAATC CCTGCCGTTG AAGCTTTGCC CGCTGTGGTG
AAGTCAGAAG GCGGTTCTAG GATTTTCTCA GCTGTGATTG GGATTGGGGC CCTCAGATCT
CACAATTTCG CTCGCATCTC CTTCGCTTCT CAGGTCTCTT CTTCTTCACC CACCCTCTTA
TCACTTCTCA GGCCTTTGCA TATTTGGTGG GGCTGTGTTA CAAGATCTGG TCCGAAAAGA
ATTCATAGCC AAAGGTTTTG ATTCACCAAG TGGCTGATGA GITTGTAGCC AAAGGTTTTG
ATTCACCAAG TGCCTGATGA GTTCTTAGCC AAAGGTTTTG ATTCACCAAC TACCTGAATC
TTGGGCATGC TTAAAAACGT GGGGTGCCCA CCATAATGAA AGGCCAGATC TCTGGTGGGG
TACTCCCAGA TTTTGTTGGG AGCACATGAA CAATGAACCC TTACCCACCC TTATATCTGA
CCCAGATGTT CTTTGACAGG AATATGAATG AGTTATTTTC TAAATCTATG GATGCAAAGA
GAACATGTTA GGAACATTGA TATTATCATC ATCATTATCA CCAATACCCC AAGACCCCTT
ACCACCTAGG AATTAGCTCC CACCAAACTT ATTTGATTAT TTTAAGCCCT TGGGAGTTGA
TCATGTAAAT TTGGCACCCA TCTGATTATG AACTGACTAG AATTTTGACA GCCTTAGAGC
CCTCAAAAGT TTACCTCTTT TGTTCAAATG GACATTGATT GTTGTATAGA GGTGGGTCAT
GAGGGCATGA GGAGACCTTT CATCCCTATT GTGGGAGCCA CAGGCCAGGC TGTTCCATGC
CAAGTGAAAG CCTACTGTTC CTGAAAATGA CTGGTGGGGA TTACAGTATG TGTATATATT
TTAATTCTTA CACGATCAGC TAAGGAGAAC TTGACTAGGT CTTGTGACTT TTGGTTAGGA
AGGAGGCCGT TGGATATCTC TAATTGTTTC TTCTTTATTA CTAATTATAT AGGGTTTGGT
CAACTTCTGT GGAAGCCAAT GTTTTGGTGG GAAGGTTGGG TGTGGTAGTT GGAGGAGTCC
ATTTGTTACC TTTGCCAGCG CGGACTATAG TAAATGCTAT TCTAAAAGTG TGGAAACGCC
CCTTGAGCCA AGGTCATCAG CTGGAAAATT CCTGAGTGGT ATATTGAAGA ACCATCCACA
CATTTTCAAT GTGGCTGCTG CAGAACAACT AGAGGAATTG GTTGCAGAGA GGAATGGTGC
ATTCGCTCGA CGTGAGCAAA GCTTGGGTTC AACTGAATTA TGCCTTCATG GGTTAGTTGC
CCGATAA
PPO-E partial coding sequence (5′ to 3′) (SEQ ID NO: 14)
ATGGAAAGTG GCGTTTGCTG TGGTAGAATC CCTGCCGTTG AAGCTTTGCC CGCTGTGGTG
AAGTCAGAAG GCGGTTCTAG GATTTTCTCA GCTGTGATTG GGATTGGGGC CCTCAGATCT
CACAATTTCG CTCGCATCTC CTTCGCTTCT CAGGGTTTGG TCAACTTCTG TGGAAGCCAA
TGTTTTGGTG GGAAGGTTGG GTGTGGTAGT TGGAGGAGTC CATTTGTTAC CTTTGCCAGC
GCGGACTATA GTAAATGCTA TTCTAAAAGT GTGGAAACGC CCCTTGAGCC AAGGTCATCA
GCTGGAAAAT TCCTGAGTGG TATATTGAAG AACCATCCAC ACATTTTCAA TGTGGCTGCT
GCAGAACAAC TAGAGGAATT GGTTGCAGAG AGGAATGGTG CATTCGCTCG ACGTGAGCAA
AGCTTGGGTT CAACTGAATT ATGCCTTCAT GGGTTAGTTG CCCGATAA
PPO-E partial amino acid sequence (SEQ ID NO: 15)
MESGVCCGRI PAVEALPAVV KSEGGSRIFS AVIGIGALRS HNFARISFAS QGLVNFCGSQ
CFGGKVGCGS WRSPFVTFAS ADYSKCYSKS VETPLEPRSS AGKFLSGILK NHPHIFNVAA
AEQLEELVAE RNGAFARREQ SLGSTELCLH GLVAR
PPO-F partial genomic sequence (5′ to 3′) (SEQ ID NO: 16)
CTCCAACCCA TACATTATGT GGACCCTACT TCTAATTAAT TAAGATATAC ACCTCATCTT
CTTCATCCTT CACATGAATC ACCGTCAATG GCATCTTTCC TAAATCCCCA ATTCCTCACC
CACACCATCT CCTCCAACAG ACCCTTCCTC CATCGCTCTC TCATCTGCGC TCACAAACCC
GATTCCCAAT CCTCTCCAAC CCACAGGCGT CGGATCCTAA TCGGATTAGG AGGATCGCTC
CTCCTATCTG CTGCTGCTTC TTCTTCTTTA TTCTCCCGAC CCAGAACCGA TCCACTCCAT
CCAACCGTCC AATCTCAACA TCCCAACTCG TGGCCCACCA TCTTCCAAAT GGACGCGGCA
GAAGCTTCCA CGATCGACGG AGAATTCCCA TGCGTGTTGG ACTCGGTGGT CAAAGCCACG
GTCAAGAGGC CGAAGAAGGC GAAGAGCGGG GAGGAAGAGG TGCTGGTGGT GGACGGGATC
GAGGTGTATA ACAACGTGCC CGTGAAGTTC GACGTGCTGA TCAACGTGGC GGATTGGCGC
ACGTGCGGGC CCGGGTCCAG CGAGTTCGCG GGGAGCTTCG TGCACGTGCC GAGGAAGCCG
TGGGACCCGG AGGGGAAGGT GAAGACGCGC CTTAGGCTGG GGATAACGGA CCTGCTGGAA
CAGATTGGAG CTGATAGGGA TGATGAGTTC ACAGTCACTT TTGTGCCCAG GGCTGGAAAT
TATGTCAGAG TTGGAGGGGT TAGGATCGAA TACAGTTCTT GATTGGGTCA GTTCTATCTA
TGCTCAGTAT GAAAACACTT TATGTTTTTT TTCCAAATCT TGCACCATGT ATGGGCCCCA
CAAGAAATAG TGCTCACCTG AGGTGGGGCC GGTCTGCGTG AATAGACTTT GCTGTATGTG
TGTTTGAAGA TGATGGTCTT CTTTCTTGGA ATGTCTGTAT ATTGTATCTA TTGACAGTCT
TTAACAATTC CTCAGTGTTA TTTGTTTTTA ATTTTGATGA CATCTTGATC TGGCCCAATT
GGCATTAGCA TATCCTTTTA GGTGGAAGTT AGAGATGGTC GAATGAAAAG AGCCTCTGTC
AAAATCTCCA CATAAGTAGT GAAGGATCTG ATTGAACCAC AGTAAGATGA GGTTGTTGTG
GATCAAACAT AAATTGGCTC ATTATTTGAA CAACATGAGC AATGTCTGGA CGGGTGATGA
TGAAGTAAAT GAGGCTTCTA AGTGACATAT AGACCATCAT CAGCGATATG GTAGTTAGTT
TGTTAGGCGA GAAAGTATAA CCAAACCCTC CACATAATTT CTTCAATCAA TGTGAGGCCC
ATGAGCAGTC ATCTTAATCT CCAGGCCTAG AAAATATGTT AGATGTCTCA AATCTTTAAT
GTGGAAATGT TTGGCGAGGT GAGCTTTTAG ATTAGTAATT CCAGTAGAAT AAGTACCAGT
GATTACCGTG TCATCCACAT TTAGGAGAAG AATTGGAATA CCTTACCTTT TGAAGTTCTT
AGTGAGAAAA GAATGGTTAT GTGCACTCTG ATGGAAAGCA ACTTTTATAA CAGCAGTTTG
GAATTTCTCA AACTAGACAC ACTGGACTTG CTTAAGGCCA TCAGACTTAT TCAGATGGCC
TGAGGGGCAA TAATGCCAAG GAGGAGGAGA CTTTTGTACA ACCTCGTGAA GGTCCCCAAT
CCTGAGTGTC TTCCCGGTTG CCTGATCCTG CAGCACAACT AGAAGAGAAA GCAACTTTTA
AAAATCGTCC ACAAGTTGAC CAACAGAGAT AAGATTAGCA GATAAATCGT GGACATAACA
GGTATCAAGA GATGCAAAGG TTGGCTGTGA CAATTGGATA ACACAATCGT ACCAGTACTA
GCGATGGGCA AATGGTTACC ATTGGCATTT ATAGTATGCC CTTTTCTAGT GTAGGGTTCC
ATATGATGAA ATGTAGATTT GACTGAAGTC ATTTGGTTGG GTAATCCTGA ATCTGTGTAG
CATGATGACA TGGGATGAAA TGTAGGCTAG CAGGGTAAGA AAGAGTTACT GGATATTCCC
ATAGCAGAGA ATGCACTTTG TGTGCATAGG GCAATAGAGC TTTCAATGAG TTCTTTAATC
TGCTCTGGAG TCAGGGAAGG AGATTCAGAG GCAGAGACGG GCACAACATC AGTACTAGCT
TGGGCTTTCA ACGTTGTTGG GCCTGGACCA TTACTTGGAG GGGTAGATGT GTCTTGCATA
CTAGACGTTA GGAGTGGGGA ATCTTGGCTG AGAGAGGACA GGGATTGCAA GCTATGATGC
ACAGATACCA CAAAATGGGC AGCGTATCGT GTCGTATCGT ATCCGATGCT TCACTTTTTA
TGTGTAAAAT CTATGTCCAT GTCATGTCTA CGGCGTGTCC TTGCCGTATC C
PPO-F partial coding sequence (5′ to 3′) (SEQ ID NO: 17)
ATGGCATCTT TCCTAAATCC CCAATTCCTC ACCCACACCA TCTCCTCCAA CAGACCCTTC
CTCCATCGCT CTCTCATCTG CGCTCACAAA CCCGATTCCC AATCCTCTCC AACCCACAGG
CGTCGGATCC TAATCGGATT AGGAGGATCG CTCCTCCTAT CTGCTGCTGC TTCTTCTTCT
TTATTCTCCC GACCCAGAAC CGATCCACTC CATCCAACCG TCCAATCTCA ACATCCCAAC
TCGTGGCCCA CCATCTTCCA AATGGACGCG GCAGAAGCTT CCACGATCGA CGGAGAATTC
CCATGCGTGT TGGACTCGGT GGTCAAAGCC ACGGTCAAGA GGCCGAAGAA GGCGAAGAGC
GGGGAGGAAG AGGTGCTGGT GGTGGACGGG ATCGAGGTGT ATAACAACGT GCCCGTGAAG
TTCGACGTGC TGATCAACGT GGCGGATTGG CGCACGTGCG GGCCCGGGTC CAGCGAGTTC
GCGGGGAGCT TCGTGCACGT GCCGAGGAAG CCGTGGGACC CGGAGGGGAA GGTGAAGACG
CGCCTTAGGC TGGGGATAAC GGACCTGCTG GAACAGATTG GAGCTGATAG GGATGATGAG
TTCACAGTCA CTTTTGTGCC CAGGGCTGGA AATTATGTCA GAGTTGGAGG GGTTAGGATC
GAATACAGTT CTTGA
PPO-F partial amino acid sequence (SEQ ID NO: 18)
MASFLNPQFL TPTISSNRPF LHRSIICAHK PDSQSSPTHR RRILIGLGGS LLLSAAASSS
LESRPRTDPL HPTVQSQHPN SWPTIFQMDA AEASTIDGEF PCVLDSVVKA TVKRPKKAKS
GEEEVLVVDG IEVENNVPVK FDVLINVADW RTCGPGSSEF AGSFVHVPRK PWDPEGKVKT
RLRLGITDLL EQIGADRDDE FTVTFVPRAG NYVRVGGVRI EYSS
PPO-G genomic sequence (5′ to 3′) (SEQ ID NO: 19)
ATGGCATCTC TTTCCTTCCT TTCCACCACC CAAACCGCTC CCCTCCACCA CCCCAGAAAG
CCCCATCAAC CATCTCCAAC CTGCATTGCC CGCACAAGGC GTTTCCACAC TTCTTGCAAT
AGTACCAGCA GTAACAACAC CACTCCTAAT ACTACTAATA CTACTGATAC TTCCACCAAG
AGAGGAGTAC CCGGATCCGG ATCCGGATCC GGATCTCCTC TCCGGTTGGA CAGGCGCAAT
GTTCTCTTAG GCCTAGGAGG CCTCTATGGT GCAACCAGCC TCCCAGGCCG GGAGAAAATT
GCGCTTGGAG CGCCAATATC TCCACCAGAC CTCTCCAAAT GCCACCTTGC CGACGGCGGC
ACCGGCGTCG GCAATGTTCA ATGCTGCCCT CCTTACTCTA GTGACACTGT ACCTATTGAC
TATCAGTTCC CGGCATCATC AAAGCCGCTG CGGATTCGCC GCCCAGCTCA TTTGCTAGAG
AAGGAGGAGA TTGAGAAGTA TAAGGAGGCA ATAGCCAAGA TGCGGGAACT GACTACCACG
GATCCGAGTG ACCCGAGAGG GTGGATGCAG CAGGCCAATG TCCATTGCCA GTACTGCAAT
GGCGCCTATG ACCAGGTTGG CTACGACAAT GTCCGGCTGC AGGTGCACTT CAGCTGGCTG
TTCCTGCCAT GGCACCGCTG GTACCTCCAT TTCTATGAGA GAATTCTGGG GAACCTCATC
GGCGACGATA GCTTCGCGCT CCCTTACTGG AATTGGGACA CCCCACTTGG GATGTACGCA
CCAAGTATAT TCGTCGACAC CACCTCGTCG CTCTACGATG AGAATCGTAA TCTTAGCCAC
TACCCGCCGG CGGTGCTCGA CTACAAGTAT GCCTATGGTG ACGCCGTCCC ATCCACAGAG
GAAGCAGTGC AAGAGGTGAG CTTTGACACT ATCTTAATTT GGAGCTTTTG AAAACAAAAA
TCAATAGACA TGGAAGTATA GATAATCCAT GCCTCATAAG AAGTATAGAT GATCCATGTC
TATAGAGTTT TCTTTTTCTA AAGAAGTGAT GTGTGCTAAC ATGGTCTCTT GTGAGGTTAG
GTGTAGGACT AAGACATTCT TCTTTAAAAA AAATGTGGTT AATTAAGGAG TGTGAGTGCT
TTATGTGACC CTTTAATATT AACTATGACT TTTGGTTAAA TGCATGTGTA AAAAGTATTA
GAACACCTTT TCATTTCTTC GTCTTTTGGT GTTGTGTATC ATGGGGACGA CTTTTTGTAC
AACAAGTGGG ATATTGTAAT GAGTGAAAAA TCTAATGATT TTCCATTTAG CATACTTTTC
TCTTATTTGA TGCCACACTT TCTCATATGG AAATGAGAAA AAATGAACTA ATAGGCCTTG
GTATAGAAAA GATTTCAATT TCCTATCAAA GAAGTAGATA AACAGCAGGT TTTCTGTGAT
TTTTTTTTAA ATTTTTTTGC TACAGGTTGT TAATCAAAAC CTATTGGAGC TGAGCAAGAC
GTACAAGGAG AGCTTGACCC TGCCCGAGCT GTTCATGGGG GACCCAATAA GGGCCGGGGA
GGCAACTGAG ACAGACACGG AAGCCTCATC AGGAAGGCTT GAGATCATAC ACAATGGGGT
GCACCAGTGG ACCGGGCCAG ACACGGTCCC TTACATGGAC ATGGGCAACT TCTACTCGGC
GGGCCGAGAC CCTATCTTCT ACTGCCACCA CTCGAACGTG GATCGGATGT GGCAGATTTA
CAGGTCCATG AGGGGCAACA AGACCGAGTT CAAGGATGAT GACTGGCTCA ATTCCTCCTT
CCTCTTTGTA GATGAGAACA AACAACTAGT GAAAGTGAAG GTAGCACTGC AATGACATTA
TATTAAAGCA CTCTCTACAT CATAGTTTTG GTTTTGCTTT TTTCTCTTTC CACACCCAAG
CCATACATTG CATCTGTCCT TTTTTGGTTT TTCTAGTGGA TCACTGAAGA AGATGCCTAG
TCTATGCCAC CAAGCCCACT ATAGAAGACA GAGACCTCAC CCACCCAAGC AATAGCTGGT
CAAGAAAGTT CCTCTTTTTA ATTCATAAAG AAAAAAAACA GATGGTCCCA ACGAATCTAA
AATTATAATT AAAATACAAG TGGATGATTA ATTTTCATGA TATTCGCCTA GGATCCTTTT
GCACATGGAC AGTGATTTCA GACTTTTTAT ACTTTTCAAT AGAAGTAGTT AGATGAACAA
ATTAAACAAT TAAGGATGTA TAATTTGTTG TTGATGATAT TTGAACATTT GTGGGTGGCA
CAGGTGCAGG ACTGCTTCAA CCCCCTCAAA CTAAAATACT CCTACGAAGA AGTGGAGCTA
CCATGGGCCG AGGTGGGTAT CCGCAAGAAA CTGACCAAGG TCACGGCCAA GGCCAAGACA
CTGTCCTTGA TCAAAGTAAG CGAGTTCGGG TCCGATCCGA AGACCCTTGA CAAGGCCACC
ATCCGGGTCC TGGTGACCCG GCCCAAGAAG TCAAGATCCA AGACCGAGAA GGAGGGTGCT
GTGGAGGTTC TTATCATCAA GGGCATCCAG GCACCCATCT TCGAGCCATC TAGGTTCGAC
GTCTACATCA CTACCCCCTA TGAGGGTGAC CTAGTAGCCC CGAGCCTTGG TGAGTTTGCA
GGCAGCTTCA CAAAGCTGCC CCACCATGGC ATACAGGTGC AGTGGGAAGG GACCAAGACC
AAGAAGTCTA AGCTCAAGCT TGGTATCAAC AACTTGCTGG AGGATATTGA TGCTGAGGGG
GCTGAGAAGC TGGTGGTGTC CTTGGTCCCA CGTTTGGGGC AGGTTACTGT TGGTGGTGTA
AGCATTGACC TCCTGAACAC TTGA
PPO-G coding sequence (5′ to 3′) (SEQ ID NO: 20)
ATGGCATCTC TTTCCTTCCT TTCCACCACC CAAACCGCTC CCCTCCACCA CCCCAGAAAG
CCCCATCAAC CATCTCCAAC CTGCATTGCC CGCACAAGGC GTTTCCACAC TTCTTGCAAT
AGTACCAGCA GTAACAACAC CACTCCTAAT ACTACTAATA CTACTGATAC TTCCACCAAG
AGAGGAGTAC CCGGATCCGG ATCCGGATCC GGATCTCCTC TCCGGTTGGA CAGGCGCAAT
GTTCTCTTAG GCCTAGGAGG CCTCTATGGT GCAACCAGCC TCCCAGGCCG GGAGAAAATT
GCGCTTGGAG CGCCAATATC TCCACCAGAC CTCTCCAAAT GCCACCTTGC CGACGGCGGC
ACCGGCGTCG GCAATGTTCA ATGCTGCCCT CCTTACTCTA GTGACACTGT ACCTATTGAC
TATCAGTTCC CGGCATCATC AAAGCCGCTG CGGATTCGCC GCCCAGCTCA TTTGCTAGAG
AAGGAGGAGA TTGAGAAGTA TAAGGAGGCA ATAGCCAAGA TGCGGGAACT GACTACCACG
GATCCGAGTG ACCCGAGAGG GTGGATGCAG CAGGCCAATG TCCATTGCCA GTACTGCAAT
GGCGCCTATG ACCAGGTTGG CTACGACAAT GTCCGGCTGC AGGTGCACTT CAGCTGGCTG
TTCCTGCCAT GGCACCGCTG GTACCTCCAT TTCTATGAGA GAATTCTGGG GAACCTCATC
GGCGACGATA GCTTCGCGCT CCCTTACTGG AATTGGGACA CCCCACTTGG GATGTACGCA
CCAAGTATAT TCGTCGACAC CACCTCGTCG CTCTACGATG AGAATCGTAA TCTTAGCCAC
TACCCGCCGG CGGTGCTCGA CTACAAGTAT GCCTATGGTG ACGCCGTCCC ATCCACAGAG
GAAGCAGTGC AAGAGGTTGT TAATCAAAAC CTATTGGAGC TGAGCAAGAC GTACAAGGAG
AGCTTGACCC TGCCCGAGCT GTTCATGGGG GACCCAATAA GGGCCGGGGA GGCAACTGAG
ACAGACACGG AAGCCTCATC AGGAAGGCTT GAGATCATAC ACAATGGGGT GCACCAGTGG
ACCGGGCCAG ACACGGTCCC TTACATGGAC ATGGGCAACT TCTACTCGGC GGGCCGAGAC
CCTATCTTCT ACTGCCACCA CTCGAACGTG GATCGGATGT GGCAGATTTA CAGGTCCATG
AGGGGCAACA AGACCGAGTT CAAGGATGAT GACTGGCTCA ATTCCTCCTT CCTCTTTGTA
GATGAGAACA AACAACTAGT GAAAGTGAAG GTGCAGGACT GCTTCAACCC CCTCAAACTA
AAATACTCCT ACGAAGAAGT GGAGCTACCA TGGGCCGAGG TGGGTATCCG CAAGAAACTG
ACCAAGGTCA CGGCCAAGGC CAAGACACTG TCCTTGATCA AAGTAAGCGA GTTCGGGTCC
GATCCGAAGA CCCTTGACAA GGCCACCATC CGGGTCCTGG TGACCCGGCC CAAGAAGTCA
AGATCCAAGA CCGAGAAGGA GGGTGCTGTG GAGGTTCTTA TCATCAAGGG CATCCAGGCA
CCCATCTTCG AGCCATCTAG GTTCGACGTC TACATCACTA CCCCCTATGA GGGTGACCTA
GTAGCCCCGA GCCTTGGTGA GTTTGCAGGC AGCTTCACAA AGCTGCCCCA CCATGGCAGT
GGGAAGGATA CAGGTGCGAC CAAGACCAAG AAGTCTAAGC TCAAGCTTGG TATCAACAAC
TTGCTGGAGG ATATTGATGC TGAGGGGGCT GAGAAGCTGG TGGTGTCCTT GGTCCCACGT
TTGGGGCAGG TTACTGTTGG TGGTGTAAGC ATTGACCTCC TGAACACTTG A
PPO-G amino acid sequence (SEQ ID NO: 21)
MASLSFLSTT QTAPLHHPRK PHQPSPTCIA RTRRFHTSCN STSSNNTTPN TTNTTDTSTK
RGVPGSGSGS GSPLRLDRRN VLLGLGGLYG ATSLPGREKI ALGAPISPPD LSKCHLADGG
TGVGNVQCCP PYSSDTVPID YQFPASSKPL RIRRPAHLLE KEEIEKYKEA IAKMRELTTT
DPSDPRGWMQ QANVHCQYCN GAYDQVGYDN VRLQVHFSWL FLPWHRWYLH FYERILGNLI
GDDSFALPYW NWDTPLGMYA PSIFVDTTSS LYDENRNLSH YPPAVLDYKY AYGDAVPSTE
EAVQEVVNQN LLELSKTYKE SLTLPELFMG DPIRAGEATE TDTEASSGRL EIIHNGVHQW
TGPDTVPYMD MGNFYSAGRD PIFYCHHSNV DRMWQIYRSM RGNKTEFKDD DWLNSSFLFV
DENKQLVKVK VQDCFNPLKL KYSYEEVELP WAEVGIRKKL TKVTAKAKTL SLIKVSEFGS
DPKTLDKATI RVLVTRPKKS RSKTEKEGAV EVLIIKGIQA PIFEPSRFDV YITTPYEGDL
VAPSLGEFAG SFTKLPHHGS GKDTGATKTK KSKLKLGINN LLEDIDAEGA EKLVVSLVPR
LGQVTVGGVS IDLLNT
PPO-H genomic sequence (5′ to 3′) (SEQ ID NO: 22)
ATGTCTCTTC ATCATCTAAC GACCACCACC CCACTTTCAA CTTCATCCCC CCACCAAAAA
ACTCAATTCC AAAAGCTTGA CAAAAAGCAC TTATCTGTTT ATACAAGCAG AAGGACAACA
GGGTGGCCAA GTAGTATTAG AAGTAGTAGT ACTAACAGCA ATGGTGATGA GACTATTGCT
GGTGAAGAGC AATCTGCTTC TTCGAAACGG GTCGACCGGC GAGACGTCCT ACTCGGCCTG
GGAGGGCTGT ATGGTGCAGC TGGTCTCGCC GGCCAGGCCC TGGCGTCGCC GGTGACCATC
CCCGACCGGA ATGCCTGCGG CATTGCAACG TCCCCAGTAC TCCCCGGGCC AATTTATTGC
TGCCCACCAG AGAAAGTAAG GACTGCTCCT ATCGTCCAGT GGCAGTCTTC CAACAAGGGT
CCACTCCGGG TCAGGAAACC GGCCCAGGAG ATGAACAAGG ACGAGGTGGC CAGGTTCAAA
GCGGCAGTGC AGGCTATGAA AGATCTGGAT CCGGAGGACC CATGGCACTT CGACCAGCAG
GCGAAAATCC ACTGCGCCTA CTGTAACGGG GCTTACAAGC AGGTGGGCTT CGACGTCCCT
CTTCAGGTCC ACTTCAGCTG GCTCTTCCTC CCTTGGCACC GCTGGTACCT CTACTTCTTT
GAGAGGATAC TTGGAAAGCT GATCCAGGAC GAGAGCTTTG CTCTCCCATT CTGGAACTAT
GACAGGCCGG AGGGGATGTA TATGCCGAGC ATCTACGTCG ACCCATCCTC GTCCCTCTAC
AACTCCAAAA GGAACCCGAA ACATCTGGAA TCGCTCCTGG ATTTCAATTA CAGCTACGAT
GCAGATGGCT TAACGGGGAC GGAGAAGGAG GTCATTCAGG CAAACCTAGT GGAACTGCGG
ACCATGTACG ACAGTGGCAT TCCCACGCCA GAGCTGTTCA TGGGTGACCC GGTTTCCGCA
GGTGAGCTGA CAGACTGATA TTGAGACCCG ATTTTAATTA AAACTTTACA ACGGATTAAG
TAATTTCAAC TTGCATCATC TATGCGGATC AACGTGGGTT GACTTGCATA AGTTGCACTC
AATGACGGTC CATTTGGTTT AATTCAATCT CCCTGTATTA CACGGGTCAA AAAGGTTTTT
ATCCCCATGG CATACAGATT CAATCCGAAT TGGTAGCATG GGATTATACA TTGGTATGTA
TTGTGTGGTT GACTGTGCAT TGGATGAAAA ACTTCCAAAT CACGTTAAAA GGTGAGACCC
AACTGGATCT TTTCATATGG GACCTTCAGT GCGCTTACTG TGTGAAATCT TTGCACCCGA
ACAAATTCAT TTGTATATTA AAACTATATT TAGGTTAAGT TATAGGGAAA ATTTTAAAAC
CCAACTATAA TTCAAGCCTT CGTTTCAAAT ATGTTGGAGT CTGTCTACAA AAATTCTGTT
TATTTATACC AATAATCGAC AGTCAAATAT CTCCGTAAGT TGCACTTTTT TACCTCTATT
GCTACCACTT CCATAAACAT ATAACCACAG AGTTATTTAA TATCTTTTCA TTGGCGACCT
GGTTTGTGCA GGAGAGGAGA CCGCGGAGGA CAACTCGTCC GGCTCGCTCG AGAGGTTCCA
CAACACGGTG CACATGTGGG TTGGGAGACA CAAGAATGAT GCGACCGAGC CCTACGTGGA
CATGGGTGAC TTCTCCACCG CGGCTAAGGA CATGCTCTTC TACGCGCACC ATTCCAATGT
GGACCGCTTG TGGGAAATCT ACAGGACGCG CCGAGGGAAG AAGTTGGAGT TCAAAAGCAA
CGACTGGCTC AATGCCGAGT TCATCTTCTT CGACGAGAAT AGACAGGTGG TCAAAGTAAA
CGTGAACGAT TCTCTAAGTA CACTCGATCT GGGGTACACC TATAAGGATA GTGTTCCCAC
TCCATGGTTG GAACCTGCAC GTCCTAAAAG ACCAGCAGCT AAGCCTAGGT CCGGGTCCTT
CTCTATGGTC CCTGTGACCG AGTTTGGGAC TGAACCCAGA GCTCTCGTGG ACGCGCCTGT
CCGGGTCTTG GTCTCCAGAC CGAAGACTAG CCGTAGCCAG GATGAGAAGG AGGACGAGAA
CGAAGTCCTC GTTGTCGATG GAATTGAAGT GGTAGAGGAA GGGGCTGTCA GGTTTGATGT
CTTTCTCACC TCCCCGTTTG GGAACTTTGC AGGACCCGAC TATGGGTTGC CTGCAGGGAG
CTTCGTGAAG CTGCCCCATA AACATAAGGC AGGGAGCAAG CAGAGGAAGG CGAAGCTGAA
GCTGGGGATT ACGAAGCTGT TGGAGGACCT CAAAGCTGAT AACGCGCAGA AGCTGGTTGT
GACCTTTGTG CCCCGGACTG GGAGTGTGAA CATTGGAGGA GTACATGTGG AACTGTTCAA
GACTGATAAT TAGATGTCTG GTTTGGGTCT CGGTCGTTTT AGTATGGATG GTCAGTTGTT
GTTGCCGTCG CTTGCTTTGC TTTGAGTTAA TAAGATGTTG AATCTAATTT CAACCCAATC
GAGTATATGG TATGCATGTG CTGTACGTAT CTTTAATCAA GTCTTATCAA TGCTGTTTCT
TGTCAGCTTC CATCAAGTAG TTGATTCCTT TTCTTTCTTT TTCATTTTGA AAAGTAGAAC
ACATTGCAGA CAAAATAAAA TTAGAGGCCC TCTTTGGATC TTAGGACTGG TGATGGTATA
GCACAAAACA ACTTGCAGTT GTTCTTGGTG GCCTATTCAA ATTCATGAAC CCTCATTTAT
GCAAAAACTT AAATACAGTC AGGTTTAAGA GGACTCCAAA GGAATATGGA AACAGAAACA
ATTACACTAA TAAAAAAAGG GTATTTAGAA TTGAGAAATA TTCAAGTGAG ATCTGCCGTG
GGAACTAAGG GTCGAAGTGG GACCCACCGA TGAGGCTATC ACCGAATCTA AATTTTGCAA
TAGATGGGGG TCCCCCTCAT TGTCACTCAC CCTTAGGTTT GGCACTCAAG CAAATTTACT
ATTCAAACTT GATATTTGAG GTGGCCATAC TGGAAAATTT TCATATCCCG TGATCTTAAT
CTTGTTGAGA GAGAAGGAAA GAAAGAAACT ATAAATACAA CGGAAAACCA ATTTATTGCA
TTCTACTAAA CTGCATTGCA TCAACCAATA TAAATAGATA AGCCCCAAGC AGCCCTACTA
AATAAAGTCC GAACAAAGAT AGGAAACACA AGCACACTAA CAACAGTATA TTCCTAGAAA
TATTCCTAAA GTACATATAT AGCTTCACTG GCCCAATCCT TAGTGTGCCT GTTGAATCAA
CAATGACTTC AACAATCTGC ATATGGAACC CACACAGGGT CCCACAGAAA ATTTACATGA
AACTTCAAAA GTGGGTCTAA GTGGGCCAGA AGGGACTAAG TGGGTAAATA AGGGTCATAT
TAAATATGCT GACTTAAATA AATAAGATTA TTGATGATAA ATGAAGTTCA TGATCGTGAC
AATTACCCTA TTAAACAAAT AAACTAAATC ATGAGGATGA ATTAATTTGA ACAGGCTGAA
CTATGTGATG ATGGATTTAG TTAAAATGAA CTAAGTGATG ATGAATTAAA TTAAAATGGC
CTAAGTGATG ATGAATTAAA TTAAGATAAA AGAAAAACGG AAGAATCTCA CGGGTATTTT
AATAAAAATC AGGGTCGAAT AATTGGAGAT TTGCCCGTTG GTTTATTGGC TTGAGTCCAA
AGTGGGTATA GTCTGGGCTG ATGCTAATGA CTGGGCTAGG TATAGGTCCG ACTGGGTTGC
GAATTGAGTG GGTATGGTTT GAGTAGGATG TTGGTTTGAT GGGCTCGGGT TGGTGGACCA
GATTTGAGTG GATCGAGGCA GGTGAGGCTC AGGTTGAGTG GGTACGTCCA GTGGCTGGAC
TCGAGATATG TGCTGGTGGG TTTGGTATCA GACTTGACTG GGCCCAGTCA AGTGGGTTGT
ATATAGGTAA AAGGGCCAGT GAGAGTGGTG AGTTGGTTCG GAAGGGTTAT GAGAAGGATA
AGGTTGGCTC GGTTGCAGTT GGGAAATGGG CGTAGGTTCA GTTGTTTGAG AAAGATAATG
AAGGAAGGGG GAGATGGAGA AGGTGTTTGG TGCAAATGGG TAGTGGGTGC AGAGTTTTTC
GACTAGGTGG CTGAGATGAG TGAGGATGAT CATGAGTTAC CGAACTAAAT AACCAAGTTC
AAATTTAAGA TGAATAAACG TAAACTTCAA ACTAATGCGA TATTAATGTG ATGCAAACAT
GACAAAGATG TAGGCCACGA TCAAAATCAA TAAACTTGGA TGCCAACTGC ATGCAAGTTG
ACCGATCCAC CAGATGTATG TAAAACGCAT GGTGAAAATC CGGATAAACT ACCGTGTAAA
ACAAGITGGA CAATCAAAAG GATAGAATTA TATGTCAAGA AAAGATTACC CGGTTTGCAT
ATAGTTTTGT GTGTAAGGAT AAGGTGTAGA AATATTTCTC ACGAAGAGAT CCTCCCCACT
TATGCTTTTA CCGCATGTTA CAATGTCAAG TTCGTTGTGT TTGGTTGGAG GTGCAACAGT
GTTTGGCTGG ATGGATTCGG GGTGATGTTG GGGTTGTAGC TGTGTTGGAG TTGAGTGAGG
ATTGTGGATG GAGAAGGTGA TGAAGGTTTT TGTCTCTATG AGAGGTGAGG TTAGTGGTGT
GTTGTTGTGA TTGTGGTTCG TTGATGAGTG GAGATGAGAA AGGTCGCGAG GGGGTTGTAG
AAAGAGAGTG AGAAGAGAGA GTAAATAATG GAGAGAGGTT GGCCAAGAAA AAGAGGGAGT
CAGCCATGGG AGAAAAGAAA GAAGAAAAGT GATGGCTCAT GTGGGCGTTT CTTTTTATAA
GAACAGGGCA ACAGGGCGCG CCCGAGAAAG AGAGACAAAT GGGCTGAGCG CACTCAAATG
GGTTGTTGGA TTTTGTGGGC TACTCAACAT TGCGGACCCA AAAATTTTAT ACCAGTTTTT
TTGGGGTGGG GGAAATCCTT ATATCTGGCT ACGCCTTGCA CAACTATTTC GGCCCTTTCT
ACGAAAATTT TAAGATTTAG TGGCCCAATC ATCATCTTAA CCCCATGGAG GATTGTATTT
ATCAATCTAT TGGTGGGCCT GACCTTCTCG AGAGTCCAAC ACATACTAAT TTTTAAAGTC
TGTATCTTGA GATCTGACCG TTTGGTTTTT TGAGATCTTC ATATATATAT TATTGGAGAT
TTCTCAACTG AGTGGGCATG AGAGTATATG CATATGACCC ATTTGAAGAC AAGAAAAAGG
ACTTGAGTTT TCACAGTGAC CATGGTTAGA AATTGATATC TTCCCACTGG ATTTACGAGG
CCAATATACC ACTGGAAAAC TCACAATGAA AAGATGATCA TTGAACTGAG ATTCGACCTA
AAAATGAGAC CTAGGGCTAT CCAACTGAAA ATGAAATCTA GATGATGTAA TTATCCCTTA
GATATGCATG ATGATGAATT GGCCTTAGGA TTTTAAACTA TTCATCTAAA ATATTTGAAT
GGGGCAAAAT CGAGCTGTCA CTATTATGGA TATCAGAATT GTGTATGGGG TGCTAAAATG
AGGTGCCATA ACAGGAATCA TTCTTTGGGG TGTGTGTGTA ATATTGAGGT AAGAACACCA
CCCAACCATG TGTTGGCCAA ATCGGAGCTT TGGGTAGGCT TTTAATCGAG AGTCCGTATC
CCTGAGTGGT CCCTCACACG CTTCTTGTGA GCAGGATGTG TGGAACTCTA CTTTGTGACC
CACCAATTGA TGATCCAAAC AGCTCATATT GAATGTCACT CACTTCAATG ATATATGAAT
GCATATAGTT AATAATATGT AAGATTGTGA TAAAAATCTA TTACATTAAA ATTTGAGGAT
CAGCTGTGGA GGTATGTGTT TTGTATGGCT CCAAGCATCC CACCCACGAG GAGCATGTGG
GGAGACGGTT CATAGGAGTC GGCCACGGTT ACCGAAGGTC TCTCTGTTTT TAATTGATTT
ATAGGAACTT CTCGCACACT AAGAAAAGAT TCTCCCCACC CAGTCACCCA CCTTCTAAAT
AAAACGGTCA TTTCATCCTC TCTCCATGTG CCACATGCAA CACGTGCAAT TAAAAAATGC
AGGAAAGAAG AATACATGTC CACCATTAAC TCCTGTATAT GATGATCAGA AACCAATGCA
TGTTGATGCA GGTCCCAACC TGATAGGAAT ATTGCTTAGT CTTACAATAG CTAAAGATGT
AATTAGCGGC CCTCCACCTA ACAATGCAGT CAAAAGGAGC GAGAACTAGT GATGGACCAA
GAATTITTAT TTAGGTGGGC TAAACTTTTA TTCAAGTCTG CTAACTTTAT TCACATTAAA
ATACCAAATG ATTGTGCACT TTTCCATCTC AAATCTTTTG CTTAATTGAA GGGTCTTCCC
ATTGCCTCTT CCAAATTTAG GTAATCATCA ACTCATCATC ATCAAAACAA CTAAGATCAC
ATTTCTTATA ATCCTAGTTG TTTGGGTGAG CATCTAGTTA CCCAATTTGG GTTCCCAAAG
GGATTCTATT TCAATCTAAG AAACTTGGTT GCTTGTACTT ACATAACTAT AAAAATAATT
TTCTATAGTT GGTCATGGGC TTATGGCAAA GAAGAAAAAT CGTCACTTAA ATATAACTTA
AATTATCCAA ACATATGAAC CCTAAGGCGT GGTTTATTTG TGAATTGAAA ATTAAAATGT
GCCACAATGG TGATTTGGTA ATCTTCGGAC TTGTAAATAT GCTTAGACTA GGCTCCACCG
CCCACCTTCA TGTGTATATA TATTTATTGG GGGTCTTCTC ATGTATAGTT CCCTCATCAT
ATGGCAAGGA ACACTTCCTC TTTTGGAGAT TTTCAAGTAC ATAGGGTGAG GTACACTTGT
ACTTGAAAAT CTCCAAAATG AGAAGTGTCC CTCTCCATAC CCCCTATTTA TCTATACCAT
CAATATACGT AACAACTAGG CATATACGGA TGAGGAAGGA TATGGCATTA ATAAATAAAA
GTTACGTTTT ATTTATTTTA ATGCAACGTC AATAAATGAG GGAGACTGCT ATTAGTCTTC
CATCACATGG TATAAAAAAT TCCAACCCTT TGTCACTCTT GGAGCCTTGG TATGCTGTAT
GATTGTCATC TTTTGCACAT GACCACCGTC TATCTTGTAA AGTCTATCCA CCATTGCAAT
CCACTCTATG TCTTTTTATC CTACTGTGCG GGTGATCTAG TAAATCACCT GTCATGATTC
TTTCATAAGC GATCATTAAA GTGGCTTAAT CAAGAGGTTG AGATCATGAG GTTTGAATGA
GTGTTCATGA ATGAGGAAAA GAGGATGTTC TGGTTTTATT CAAGAGAAAG AAAGTAATAG
TGATAGTGGA TCGTTGCAAA TGCATAAAAG ATGGGGGCAA TGGTTGAATT TTGATATCAT
AAAAACTTTT CACATTGGAA TAAAACCACT AATCTTTTAT TAACACAAAC ATCCTAATTT
ATATGATAGA AGAGGAATAA CATGAAAGTA ATTTAAAATT CATACTCGTG ATTACATTCA
TAACGTCTTT GACAAACTAA TCCAAATATC TTGTAAGGCC CTTCCATATT GCTACTCTCC
TATCTATCAT CGGCACAATC TCAACCAAAG TTGGCTCATT GATGGCTATA AATCTACTCC
TGAATCAATG TTAGCCCATT GTTTGATTTT GATGAATGTT TTTTCCATAC AACATCATAA
AACAATTCTT TAACGGGGAT TATTAATGAA TAATGACACC GTTTTTAAAC TGGCCAGAAA
AGAAAAGATA AAAATAAGCC CACCAGCATG TATTTAACAA AACACATGTT ATAATAACAG
TCAACATGCC CTTCGAGTCT GATGCATTTT CCCGCAGCCC ATCGTTTCTA TATATATATT
ATATCAATAC GCCATATGCA CGTGAAGACA TAGAGATGTT TTTATTTCAC CCTTAAAAAC
AACAACCCGA CAGTAATCAA TGAGACAGGC CACGCCACAT GGTGTCATCC ATCACGTGGT
CGAAAAAAGA TATTCATAAA AAAATCCATC CAGATCCGAA CACTGGCCTA TTTAAACTCG
ATCTTTTCTC CCCATCTCAA TCACCCAATA CCAACACAAG AAAACAACAC ACAAAGTCTC
TGCTCCCTTT CCCTGTTCCT CGGATTCCGA AAGATGATGT CTGTTCATCA TCCCACAACA
ACCCCACTTT CAACTTCATC CCCCCACAAA AAACACCAAT CCAAAAGACT CCACCAAAAG
CACCCAGCTG TTTATACAAG CAGAAGAACA GCAGGGTGGT GCACTGGTAT TAGAAGTAGT
AGTAACAACA AGGGTGAGAA TGCTGGTGAA GAGAAGTCTG CTTCTTCAAA AAGGATCGAC
CGGCGAGAAG TGCTCCTCGG CCTGGGAGGA CTTTATGGGG CAGCTGGTCT CGCCGGCCAG
GCCCTTGCTT CGCCGGTGGG GATCCCAGAC CGGACTGCCT GCGGCGATGC CAGCTCCGCA
AACATCTCGG GGCCACTGAA GTGCTGCCCC CCAGAGAAAG TAACTACTGC TCCGATTGTC
CAGTGGAAGG CTCCCAGTCC GGGTCCTCTC CGGGTCAGGA AACCGGCACA TGAGATGAAC
AAGGACGAGG TGGCCAAGTT CAAAAAGGCA GTGCAGGCGA TGAAAGATCT GGATCCGGAG
GACCCATGGC ACTATGACCA GCAGGCGAAA ATTCACTGCA CCTACTGCAA CGGGGCTTAC
AAGCAGGCTG GCTTCGACGT CCCTCTCCAG GTCCACTTCA GCTGGCTCTT CCTCCCTTGG
CACCGTTGGT ACCTCTACTT CTTTGAGAGG ATACTGGGGA AGCTGATCAA CGACGATAGC
TTCGCTCTCC CATTCTGGAA CTATGACAGG CCAGAGGGGA TGTTTATGCC CAGCATATAC
GTCGATCCCT CCTCGTCTCT CTACAACCCC AGACGGAACC TGGATCATCT CGAAATGCTG
CTGGATTACA ACTTCAGCTA CGACGTGAAA GGCTTGACGG GGACGGAGAA GGAGGTCATT
CAGGCCAACC TGGTCGACCT GCGGACCATG TACGACAGTG GCATTCCCAC GCCAGAGCTG
TTCATGGGTG ACCCGCTATC CGCAGGTGAG CTAACCGCAG AGGACAACTC GTCTGGTGCG
CTCGAGAGGT TCCACAACAC GGTGCACATG TGGGTTGGGA GACACAAGGA CGCGACCCCC
GACCCCTACA TCGACATGGG AGACTTCTCC ACCGCGGCTA AGGACATGCT CTTCTACGGT
CACCATGCCA ATGTGGATCG CTTGTGGGAT ATCTACCGGA CGGCCAGAGG AAAGAAGGTG
GAATTCAACA ATAGCGACTG GCTCAATGCG GAGTTCATCT TCTACGACGA GAATAAACAG
GTGGTCAAAG TCAACGTGAA GGACACTCTA AGTACACAAG ATCTGGGGTA CACCTATAAG
GATGTTCCTA TTCCATGGAT GCAACGTGCA CCTCCTAAAA GACCAGCGGC TAAGCCCAGG
TCCGGGTCCT TCTCTATGGT CCCTGTGACC GAGTTTGGGA CCGAACCCAA ATCGCTCGTT
GAAGGGCCCG TCCGGGTCTT GGTCACGAGG CCGAAGACCG GCCGTAGCCA GGAGGAGAAG
GAGGATGAGA ACGAAGTCCT CGTCGTTGAT GGAATTGAAG TCTTAGATGA AGGGCCAGTC
AGGTTTGATG TGTTTATTAC CACCCCGTTT GGGACGTTTG CAGGACCCGA CTATGGGTTG
CCTGCAGGGA GCTTCGTGAA GCTGCCTCAT AGACACAAGG AAGGGCACAA GCATAGGAAG
GCGAAGCTGA AGCTGGGGAT TACGAGGCTG TTGGAGGACC TCAAAGCTGA GAATGCGCAG
AAGCTGGTTG TGACCCTGGT TCCTCGAACT GGGAAAGTGA ACGTTGGAGG GATTCATGTG
GAGCACTTCA AGACTGATAA TTAG
PPO-H coding sequence (5′ to 3′) (SEQ ID NO: 23)
ATGTCTCTTC ATCATCTAAC GACCACCACC CCACTTTCAA CTTCATCCCC CCACCAAAAA
ACTCAATTCC AAAAGCTTGA CAAAAAGCAC TTATCTGTTT ATACAAGCAG AAGGACAACA
GGGTGGCCAA GTAGTATTAG AAGTAGTAGT ACTAACAGCA ATGGTGATGA GACTATTGCT
GGTGAAGAGC AATCTGCTTC TTCGAAACGG GTCGACCGGC GAGACGTCCT ACTCGGCCTG
GGAGGGCTGT ATGGTGCAGC TGGTCTCGCC GGCCAGGCCC TGGCGTCGCC GGTGACCATC
CCCGACCGGA ATGCCTGCGG CATTGCAACG TCCCCAGTAC TCCCCGGGCC AATTTATTGC
TGCCCACCAG AGAAAGTAAG GACTGCTCCT ATCGTCCAGT GGCAGTCTTC CAACAAGGGT
CCACTCCGGG TCAGGAAACC GGCCCAGGAG ATGAACAAGG ACGAGGTGGC CAGGTTCAAA
GCGGCAGTGC AGGCTATGAA AGATCTGGAT CCGGAGGACC CATGGCACTT CGACCAGCAG
GCGAAAATCC ACTGCGCCTA CTGTAACGGG GCTTACAAGC AGGIGGGCTT CGACGTCCCT
CTTCAGGTCC ACTTCAGCTG GCTCTTCCTC CCTTGGCACC GCTGGTACCT CTACTTCTTT
GAGAGGATAC TTGGAAAGCT GATCCAGGAC GAGAGCTTTG CTCTCCCATT CTGGAACTAT
GACAGGCCGG AGGGGATGTA TATGCCGAGC ATCTACGTCG ACCCATCCTC GTCCCTCTAC
AACTCCAAAA GGAACCCGAA ACATCTGGAA TCGCTCCTGG ATTTCAATTA CAGCTACGAT
GCAGATGGCT TAACGGGGAC GGAGAAGGAG GTCATTCAGG CAAACCTAGT GGAACTGCGG
ACCATGTACG ACAGTGGCAT TCCCACGCCA GAGCTGTTCA TGGGTGACCC GGTTTCCGCA
GGAGAGGAGA CCGCGGAGGA CAACTCGTCC GGCTCGCTCG AGAGGTTCCA CAACACGGTG
CACATGTGGG TTGGGAGACA CAAGAATGAT GCGACCGAGC CCTACGTGGA CATGGGTGAC
TTCTCCACCG CGGCTAAGGA CATGCTCTTC TACGCGCACC ATTCCAATGT GGACCGCTTG
TGGGAAATCT ACAGGACGCG CCGAGGGAAG AAGTTGGAGT TCAAAAGCAA CGACTGGCTC
AATGCCGAGT TCATCTTCTT CGACGAGAAT AGACAGGTGG TCAAAGTAAA CGTGAACGAT
TCTCTAAGTA CACTCGATCT GGGGTACACC TATAAGGATA GTGTTCCCAC TCCATGGTTG
GAACCTGCAC GTCCTAAAAG ACCAGCAGCT AAGCCTAGGT CCGGGTCCTT CTCTATGGTC
CCTGTGACCG AGTTTGGGAC TGAACCCAGA GCTCTCGTGG ACGCGCCTGT CCGGGTCTTG
GTCTCCAGAC CGAAGACTAG CCGTAGCCAG GATGAGAAGG AGGACGAGAA CGAAGTCCTC
GTTGTCGATG GAATTGAAGT GGTAGAGGAA GGGGCTGTCA GGTTTGATGT CTTTCTCACC
TCCCCGTTTG GGAACTTTGC AGGACCCGAC TATGGGTTGC CTGCAGGGAG CTTCGTGAAG
CTGCCCCATA AACATAAGGC AGGGAGCAAG CAGAGGAAGG CGAAGCTGAA GCTGGGGATT
ACGAAGCTGT TGGAGGACCT CAAAGCTGAT AACGCGCAGA AGCTGGTTGT GACCTTTGTG
CCCCGGACTG GGAGTGTGAA CATTGGAGGA GTACATCACC CAGCTGTTTA TACAAGCAGA
AGAACAGCAG GGTGGTGCAC TGGTATTAGA AGTAGTAGTA ACAACAAGGG TGAGAATGCT
GGTGAAGAGA AGTCTGCTTC TTCAAAAAGG TGGTCTCGCC GAGAAGTGCT CCTCGGCCTG
GGAGGACTTT ATGGGGCAGC ATCGACCGGC GGCCAGGCCC TTGCTTCGCCGGTGGGGATC
CCAGACCGGA CTGCCTGCGG CGATGCCAGC TCCGCAAACA TCTCGGGGCC ACTGAAGTGC
TGCCCCCCAG AGAAAGTAAC TACTGCTCCG ATTGTCCAGT GGAAGGCTCC CAGTCCGGGT
CCTCTCCGGG TCAGGAAACC GGCACATGAG ATGAACAAGG ACGAGGTGGC CAAGTTCAAA
AAGGCAGTGC AGGCGATGAA AGATCTGGAT CCGGAGGACC CATGGCACTA TGACCAGCAG
GCGAAAATTC ACTGCACCTA CTGCAACGGG GCTTACAAGC AGGCTGGCTT CGACGTCCCT
CTCCAGGTCC ACTTCAGCTG GCTCTTCCTC CCTTGGCACC GTTGGTACCT CTACTTCTTT
GAGAGGATAC TGGGGAAGCT GATCAACGAC GATAGCTTCG CTCTCCCATT CTGGAACTAT
GACAGGCCAG AGGGGATGTT TATGCCCAGC ATATACGTCG ATCCCTCCTC GTCTCTCTAC
AACCCCAGAC GGAACCTGGA TCATCTCGAA ATGCTGCTGG ATTACAACTT CAGCTACGAC
GTGAAAGGCT TGACGGGGAC GGAGAAGGAG GTCATTCAGG CCAACCTGGT CGACCTGCGG
ACCATGTACG ACAGTGGCAT TCCCACGCCA GAGCTGTTCA TGGGTGACCC GCTATCCGCA
GGTGAGCTAA CCGCAGAGGA CAACTCGTCT GGTGCGCTCG AGAGGTTCCA CAACACGGTG
CACATGTGGG TTGGGAGACA CAAGGACGCG ACCCCCGACC CCTACATCGA CATGGGAGAC
TTCTCCACCG CGGCTAAGGA CATGCTCTTC TACGGTCACC ATGCCAATGT GGATCGCTTG
TGGGATATCT ACCGGACGGC CAGAGGAAAG AAGGTGGAAT TCAACAATAG CGACTGGCTC
AATGCGGAGT TCATCTTCTA CGACGAGAAT AAACAGGTGG TCAAAGTCAA CGTGAAGGAC
ACTCTAAGTA CACAAGATCT GGGGTACACC TATAAGGATG TTCCTATTCC ATGGATGCAA
CGTGCACCTC CTAAAAGACC AGCGGCTAAG CCCAGGTCCG GGTCCTTCTC TATGGTCCCT
GTGACCGAGT TTGGGACCGA ACCCAAATCG CTCGTTGAAG GGCCCGTCCG GGTCTTGGTC
ACGAGGCCGA AGACCGGCCG TAGCCAGGAG GAGAAGGAGG ATGAGAACGA AGTCCTCGTC
GTTGATGGAA TTGAAGTCTT AGATGAAGGG CCAGTCAGGT TTGATGTGTT TATTACCACC
CCGTTTGGGA CGTTTGCAGG ACCCGACTAT GGGTTGCCTG CAGGGAGCTT CGTGAAGCTG
CCTCATAGAC ACAAGGAAGG GCACAAGCAT AGGAAGGCGA AGCTGAAGCT GGGGATTACG
AGGCTGTTGG AGGACCTCAA AGCTGAGAAT GCGCAGAAGC TGGTTGTGAC CCTGGTTCCT
CGAACTGGGA AAGTGAACGT TGGAGGGATT CATGTGGAGC ACTTCAAGAC TGATAATTAG
PPO-H amino acid sequence (SEQ ID NO: 24)
MSLHHLTTTT PLSTSSPHQK TQFQKLDKKH LSVYTSRRTT GWPSSIRSSS TNSNGDETIA
GEEQSASSKR VDRRDVLLGL GGLYGAAGLA GQALASPVTI PDRNACGIAT SPVLPGPIYC
CPPEKVRTAP IVQWQSSNKG PLRVRKPAQE MNKDEVARFK AAVQAMKDLD PEDPWHFDQQ
AKIHCAYCNG AYKQVGFDVP LQVHFSWLFL PWHRWYLYFF ERILGKLIQD ESFALPEWNY
DRPEGMYMPS IYVDPSSSLY NSKRNPKHLE SLLDFNYSYD ADGLTGTEKE VIQANLVELR
TMYDSGIPTP ELFMGDPVSA GEETAEDNSS GSLERFHNTV HMWVGRHKND ATEPYVDMGD
FSTAAKDMLF YAHHSNVDRL WEIYRTRRGK KLEFKSNDWL NAEFIFFDEN RQVVKVNVND
SLSTLDLGYT YKDSVPTPWL EPARPKRPAA KPRSGSFSMV PVTEFGTEPR ALVDAPVRVL
VSRPKTSRSQ DEKEDENEVL VVDGIEVVEE GAVRFDVFLT SPFGNFAGPD YGLPAGSFVK
LPHKHKAGSK QRKAKLKLGI TKLLEDLKAD NAQKLVVTFV PRTGSVNIGG VHHPAVYTSR
RTAGWCTGIR SSSNNKGENA GEEKSASSKR IDRREVLLGL GGLYGAAGLA GQALASPVGI
PDRTACGDAS SANISGPLKC CPPEKVTTAP IVQWKAPSPG PLRVRKPAHE MNKDEVAKFK
KAVQAMKDLD PEDPWHYDQQ AKIHCTYCNG AYKQAGFDVP LQVHFSWLFL PWHRWYLYFF
ERILGKLIND DSFALPFWNY DRPEGMFMPS IYVDPSSSLY NPRRNLDHLE MLLDYNFSYD
VKGLTGTEKE VIQANLVDLR TMYDSGIPTP ELFMGDPLSA GELTAEDNSS GALERFHNTV
HMWVGRHKDA TPDPYIDMGD FSTAAKDMLF YGHHANVDRL WDIYRTARGK KVEFNNSDWL
NAEFIFYDEN KQVVKVNVKD TLSTQDLGYT YKDVPIPWMQ RAPPKRPAAK PRSGSFSMVP
VTEFGTEPKS LVEGPVRVLV TRPKTGRSQE EKEDENEVLV VDGIEVLDEG PVRFDVFITT
PFGTFAGPDY GLPAGSFVKL PHRHKEGHKH RKAKLKLGIT RLLEDLKAEN AQKLVVTLVP
RTGKVNVGGI HVEHFKTDN

### Example 4—RNA Sequencing

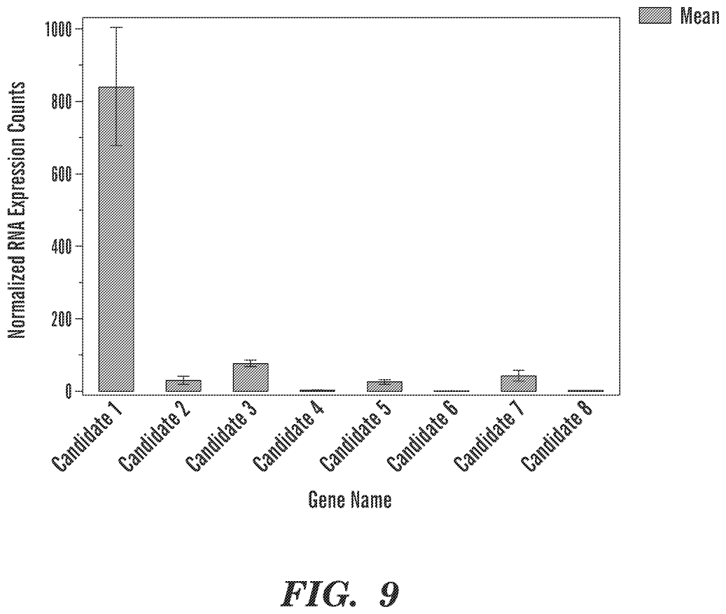

In parallel with the sequencing of the avocado genome, a detailed analysis of RNAs expressed in avocado fruit was performed using RNA-Seq to identify the PPO genes most likely associated with fruit browning. In this analysis, the total RNAs over a time course of an interrogated tissue were isolated and a polyA-enriched library sequenced by Illumina paired end next-generation sequencing (NGS) to reveal the presence and quantity of mRNA species in fruits versus leaf in response to wounding. This analysis identified 8 PPO genes with varied expression in avocado fruit (FIG. 8). The most highly expressed PPO gene identified, called “Candidate 1” in FIG. 8, was subsequently named “PPO-A”. PPO-A showed a statistically significant increase of 6.5× in expression during a timed fruit wounding experiment. Another highly expressed gene candidate was also identified in this experiment and called “Candidate 2” in FIG. 8. It was subsequently named PPO-B. Together, PPO-A and PPO-B represented 85% of PPO gene expression in whole avocado fruits and were selected as targets for genome editing and the generation of loss of function mutations. Notably, both genes were also expressed in leaf tissue (FIG. 9), which allowed the study of the effect of different PPO loss of function mutations without waiting for the gene edited avocado trees to set fruits (a process that can take 3-5 years). PPO candidates 3-8 were named PPO-C through PPO-H.

### Example 5—Generation of Avocado Calli with Stable PPO Knockouts

Since avocado is a clonally propagated woody perennial and exhibits outcrossing in nature, homozygous knockout in the Ro generation is advantageous. Ribonucleoprotein (RNP) complexes can eliminate integration of nucleic acid into the plant genome and obviate the need for backcrossing and screening of progeny. Further, the editing machinery can be controlled experimentally.

An RNP complex was delivered by polyethyleneglycol (“PEG”)-mediated transfection of avocado pluripotent cells (“PC”). To generate the RNP complex, purified Cas9 protein was combined with an avocado PPO sgRNA (single guide RNA) with the following sequence: GGUGCAUCCCAGUUCCAGAAGUUUUAGAGCUAGAAAUAGCAAGUUAAAAUAAGG CUAGUCCGUUAUCAACUUGAAAAAGUGGCACCGAGUCGGUGCUUUU (SEQ ID NO:25).

The combination of the Cas9 protein and gRNA form a ribonucleoprotein (RNP) complex targeting both PPO-A at target site GGTGCATCCCAGTTCCAGAA (SEQ ID NO:26) and PPO-B at target site GGGGCGTCCCAGTTCCAGAA (SEQ ID NO:27). Twenty nmol individual sgRNA was pre-assembled with 2 nmol Cas9 protein and incubated at room temperature for at least 10 minutes. This complex was introduced into avocado protoplasts via polyethylene glycol mediated transfection following a published procedure with modification (Woo et al, “DNA-free Genome Editing in Plants with Preassembled CRISPR-Cas9 Ribonucleoproteins,” Nat. Biotech. 33:1162-1164 (2015), which is hereby incorporated by reference in its entirety).

Briefly, protoplasts were separated from the undigested cells by filtration through a 40 μm nylon filter then centrifugated at 125×g for 5 min. Protoplasts were harvested by washing with W5 (2 mM MES, pH 5.7, 154 mM NaCl, 125 mM CaCl₂, and 5 mM KCl) twice and resuspended with MMG (0.4 M mannitol, 15 mM MgCl₂, 4 mM MES, pH 5.7) to a final concentration of 2 million cells per ml. A mixture of 1×10⁶ protoplasts resuspended in 0.5 ml MMG solution was gently mixed with RNP complex mixture and equal volume of freshly prepared PEG solution (40% w/v PEG 4000 (Sigma No. 95904), 0.2 M mannitol and 0.1 M CaCl₂)), and then incubated at 30° C. for 10 minutes in darkness. After incubation, 950 μL W5 solution was added slowly, mixed by gentle inverting, centrifuged at 120×g for 5 minutes, and the pellet was resuspended in 1 ml WI solution (0.5 M mannitol, 20 mM KCl, and 4 mM MES (pH 5.7)). RNP-transfected protoplasts were regenerated by following the procedure of Witjaksono et al., “Isolation, Culture and Regeneration of Avocado (Persea americana Mill.) Protoplasts,” Plant Cell Reports. 18:235-242 (1998), which is hereby incorporated by reference in its entirety.

Four rounds of transfection were performed. Each round of transfection had 4 technical replications with 1 million pluripotent cells per replication. Following transfection, about half of the cells were lost. Transfected PCs were embedded and cultured in liquid media (see Witjaksono et al., “Isolation, Culture and Regeneration of Avocado (Persea americana Mill.) Protoplasts,” Plant Cell Reports. 18:235-242 (1998), which is hereby incorporated by reference in its entirety) for 25 days. Microcalli were placed in solid media (see Witjaksono et al., “Isolation, Culture and Regeneration of Avocado (Persea americana Mill.) Protoplasts,” Plant Cell Reports. 18:235-242 (1998), which is hereby incorporated by reference in its entirety) to grow and proliferate. After one week in media, microcalli were counted and separated using a microscope. This early separation step was performed to prevent merging of microcalli. All individual calli were placed on solid media and in square petri dishes with 36 grids for 2 weeks before harvesting for DNA samples and sequencing.

A total of 15,000 calli were produced from four rounds of gene KO experiments using PC transfections. About 815 calli from the first three rounds of transfection and across different replications were screened for their PPO editing pattern as well as indel frequency using Sanger-based ICE (Interference of CRISPR Edits) analysis. Calli with no detection of wildtype PO allele were further confirmed by next generation sequencing.

PPO-A and PPO-B exhibited different efficiency of editing in individual calli (Table 2). Within the sequenced population, about 93% of the calli contain some level of edited PPO-A allele and about 81% of calli had more than 50% of PPO-A allele edited.

TABLE 2
Genome Editing Efficiencies of PPO-A and PPO-B

| | | PPO-A | | | PPO-B | | |
|---|---|---|---|---|---|---|---|
| | | | # of calli | # of Calli | | # of calli | # of calli |
| | Total | # of calli | with ≥50% | without | # of calli | with ≥50% | without |
| | analyzed | with edited | edited | detectable WT | with edited | edited | detectable WT |
| plate # | calli/plate | allele/plate | allele/plate | allele/plate | allele/plate | allele/plate | allele/plate |
| 2 | 93 | 79 | 70 | 35 | 65 | 1 | |
| 3 | 93 | 86 | 75 | 49 | 31 | 2 | 1 |
| 4 | 93 | 91 | 74 | 44 | 33 | 0 | |
| 5 | 93 | 88 | 76 | 42 | 31 | 2 | |
| 6 | 93 | 88 | 86 | 53 | 46 | 9 | 2 |
| 7 | 93 | 80 | 74 | 50 | 47 | 5 | 1 |
| 8 | 93 | 89 | 74 | 35 | 24 | 2 | |
| 9 | 93 | 88 | 74 | 40 | 25 | 2 | |
| Total | 744 | 689 | 603 | 348 | 302 | 23 | 4 |
| Calli | | | | | | | |
| Avg % | | 93% | 81% | 47% | 41% | 3% | 0.5% |

Further analysis indicated that about 50% of calli contain no detectable wildtype (“WT”) allele. Several individual calli showed single base pair indel edits, leading to predicted protein truncation. Compared to PPO-A, PPO-B editing efficiency was much lower, likely due to mismatches in two nucleotides in the PAM-distal region of the sgRNA for PPO-B. Even though 41% of total population of protoplasts contain at least one edited PPO-B allele, only three percent of the 744 calli analyzed contained more than 50% edited PPO-B alleles (most likely to be heterozygotes). Four calli were identified that contained no detectable wildtype PPO-B allele in addition to efficient PPO-A knockout. Three of these calli contained a 1-nt insertion and the fourth one contained a 24-nt deletion. A total of 50 individual calli were chosen, including highly homozygous lines (Table 2) as well as calli that contain higher editing frequencies of both PPO-A and PPO-B. One heterozygous PPO-A line was also included for potential control when phenotyping the regenerated plants. These lines were further characterized and regenerated into avocado plants. FIG. 7 shows the timeline and the steps to regenerate these selected calli.

Protoplasts were induced to regenerate into embryogenic calli as described in Example 1 and sequenced to verify editing of the target gene sequences. PPO edited avocado callus was screened by sequencing to identify lines with gene edits in PPO-A, PPO-B, or PPO-A+PPO-B. No lines were identified with edits only in PPO-B. Twenty PPO edited avocado lines were identified with useful and interesting knockout phenotypes as shown in Table 3.

TABLE 3
Genotype of Twenty Avocado Calli Lines Edited at the PPO-A and PPO-B Genes

| | | | PPO-A | | | PPO-B |
|---|---|---|---|---|---|---|
| | PPO-A | | Editing | PPO-B | | Editing |
| Line No. | KO* | PPO-A Editing Pattern | Score | KO* | PPOB Editing Pattern | Score |
| 1 | x | I1/D2/D3 | 96% | x | I1/WT/D1/D2 | 72% |
| 2 | x | I1/D1/WT/D3 | 75% | x | I1/WT/D33/D4/D3 | 59% |
| 3 | x | D4/D7/D1/D5 | 90% | x | I1/D1 | 99% |
| 4 | x | D13/I1/D6/D5/D8/D1 | 99% | x | I1/WT | 53% |
| 5 | x | I1/D11/D2 | 95% | | WT | NA |
| 14 | x | I1/D1/D14/D28/D4 | 96% | | WT | NA |
| 27 | x | I1 | 96% | x | WT/D26/I1/D32 | 75% |
| 28 | x | D20/I1/D2/D4/D24 | 82% | | WT/I1 | 36% |
| 29 | x | I1/D2/D20 | 97% | | WT | NA |
| 34 | x | I1/D5/D4/D12 | 91% | x | I1/D4/D5/D26/I1D10 | 97% |
| 67 | x | I1/D1/D2/I2 | 94% | | WT | NA |
| 44 | x | I1/D8/D1/D14 | 97% | | WT | NA |
| 48 | x | D24 | 100% | | WT | NA |
| 46 | x | I1/D1/D14/D28/D6 | 90% | x | I1/WT | 71% |
| 95 | x | I1/D16/D1/D24/D17 | 93% | | I1/WT | 22% |
| 100 | x | I1/D21/D1 | 98% | | I1/WT | 25% |
| 76 | x | I1/D2/D4/D2/D10/D4 | 95% | | I1/WT | 17% |
| 64 | x | D1/D3/I1/D2 | 91% | x | D26/WT | 69% |
| 52 | x | I1/D2/D3 | 95% | | WT/I1/I11/I14/D28 | 26% |
| 15 | x | I1/D5/D2/D4 | 95% | | WT/I1/D9/D25 | 39% |

“I”—insertion;
“D”—deletion;
“wt”—wild type;
Number following notation is base pairs;
“x” denotes lines with an editing score of 50% or higher

As PPO edited avocado lines were produced by DNA-free mediated transfection, there was no plant pest DNA (i.e., selectable markers, heterologous promoters and terminators, T-DNA sequences, etc.) present to screen for.

### Example 5—PPO Activity in Genome Edited Avocado Calli

Calli from 50 PPO gene edited avocado lines were sequenced at both the PPO-A and PPO-B gene locus to determine editing patterns. Knockout scores for seven such lines are shown in Table 5. Note that the editing patterns can sometimes suggest a chimeric nature of editing that is typical in gene editing experiments.

Mutations from calli with genome edits identified in PPO-A and PPO-B are shown in Table 4 and also in FIGS. 12A-I where they are aligned with the wildtype (WT) sequences for PPO-A (SEQ ID NO:28) or PPO-B (SEQ ID NO:50). Dashes in Table 4 indicate deletions and are named according to the number of deleted nucleotides (e.g., D4 indicates a deletion of 4 nucleotides). Inserted nucleotides are indicated in italics and named according to the number of inserted nucleotides (e.g., I1 indicates an insertion of 1 nucleotide).

TABLE 4
Genome Edited Mutations in PPO-A and PPO-B

| Gene | ID | Sequence (5'-3') | SEQ ID NO: |
|---|---|---|---|
| PPO-A | WT | GGTGCATCCCAGTTCCAGAAGGG | 28 |
| PPO-A | I1 | GGTGCATCCCAGTTCCAAGAAGGG | 32 |
| PPO-A | D1 | GGTGCATCCCAGTTCC-GAAGGG | 33 |
| PPO-A | D1-2 | GGTGCATCCCAGTTCCA-AAGGG | 34 |
| PPO-A | D2 | GGTGCATCCCAGTTC--GAAGGG | 35 |
| PPO-A | D2-2 | GGTGCATCCCAGTT--AGAAGGG | 36 |
| PPO-A | D3I1-2 | GGTGCATCCCAGTT---TGAAGGG | 37 |
| PPO-A | D3 | GGTGCATCCCAGTT---GAAGGG | 29 |
| PPO-A | D4 | GGTGCATCCCAG----AGAAGGG | 38 |
| PPO-A | D4-2 | GGTGCATCCCAGT-----GAAGGG | 39 |
| PPO-A | D5 | GGTGCATCCCAG-----GAAGGG | 40 |
| PPO-A | D7 | GGTGCATCCCAGTTC-------G | 41 |
| PPO-A | D6 | GGTGCATCCCAG------AAGGG | 30 |
| PPO-A | D8 | GGTGCATCCCAGT--GG | 42 |
| PPO-A | D11 | GGTGGAT------------AAGGG | 43 |
| PPO-A | D12 | GGTGC-------------GAAGGG | 31 |
| PPO-A | D13 | GG--------------AGAAGGG | 44 |
| PPO-A | D14 | GGT--------------GAAGGG | 45 |
| PPO-A | D20 | GGTGCA--------------------AGCAAATGTCTCATCTCC | 46 |
| PPO-A | D24 | GGTGCA------------------------AATGTCTCATCTCC | 47 |
| PPO-A | D28 | GG----------------------------AATGTCTCATCTCC | 48 |
| PPO-A | WT | GGTGCATCCCAGTTCCAGAAGGGGAGAGCAAATGTCTCATCTCC | 49 |
| PPO-B | WT | GGGGCGTCCCAGTTCCAGAAGGG | 50 |
| PPO-B | I1 | GGGGCGTCCCAGTTCCAAGAAGGG | 52 |
| PPO-B | D1 | GGGGCGTCCCAGTTCC-GAAGGG | 53 |
| PPO-B | D2 | GGGGCGTCCCAGTTC--GAAGGG | 54 |
| PPO-B | D3 | GGGGCGTCCCAGA---AGAAGGG | 51 |
| PPO-B | D4 | GGGGCGTCCCAG----AGAAGGG | 55 |
| PPO-B | D5 | GGGGCGTCCCA-----AGAAGGG | 60 |
| PPO-B | D26 | GGGGCGTCCCAGTTCC--------------------------CCAA | 56 |
| PPO-B | D32 | GGGGC--------------------------------CATCCCCAA | 57 |
| PPO-B | D33 | GGGGC---------------------------------ATCCCCAA | 58 |
| PPO-B | WT | GGGGCGTCCCAGTTCCAGAAGGGGATGGCGAAGGACTCATCCCCAA | 61 |
| PPO-B | I1-2D10 | GGGGCGTCCCAGTTCA----------TGGCGAAGGACTCATCCCCAA | 59 |

To determine the level of enzymatic activity in knockout (KO) mutants, 0.2 mg of calli material was sampled from lines that included a range of PPO-A KO scores (51%-100%) and PPO-B KO scores (0%-99%) (Table 5). A KO score indicates the proportion of cells that have either a frameshift or at least a 21 nt indel when being in-frame. This score is a useful measure to estimate how many of the contributing indels are likely to result in a nonfunctional KO mutant of the targeted gene. A KO score higher than 90% is considered a homozygous mutant. Scores below 90% suggest either heterozygous or partial KO chimerism in the calli. D=deletion followed by the number of deleted nucleotides. I=insertion followed by the number of inserted nucleotides. WT=wild type or unedited sequence pattern.

TABLE 5
PPO-A and PPO-B Editing Pattern and Knockout
Score for Selected Edited Lines

| | PPO-A | | PPO-B | |
|---|---|---|---|---|
| | Editing | KO | | KO |
| Calli Line # | Pattern | score | Editing Pattern | score |
| 33 | D2/D3/D4/I1 | 82% | WT/I1 | 29% |
| 34 | I1/D5/D4/D12 | 91% | I1/D4/D5/D26/I1D10 | 97% |
| 10 | I1/WT/D20 | 59% | WT | 0% |
| 36 | I1/D1 | 98% | WT/I1 | 25% |

| | | |
|---|---|---|
| Calli Line # | PPO-A | PPO-B |

| | | | | |
|---|---|---|---|---|
| 42 | I1/D5/D4/D12 | 64% | I1 | 99% |
| 48 | D24 | 100% | WT | 0% |
| 50 | I1/WT | 51% | WT | 0% |

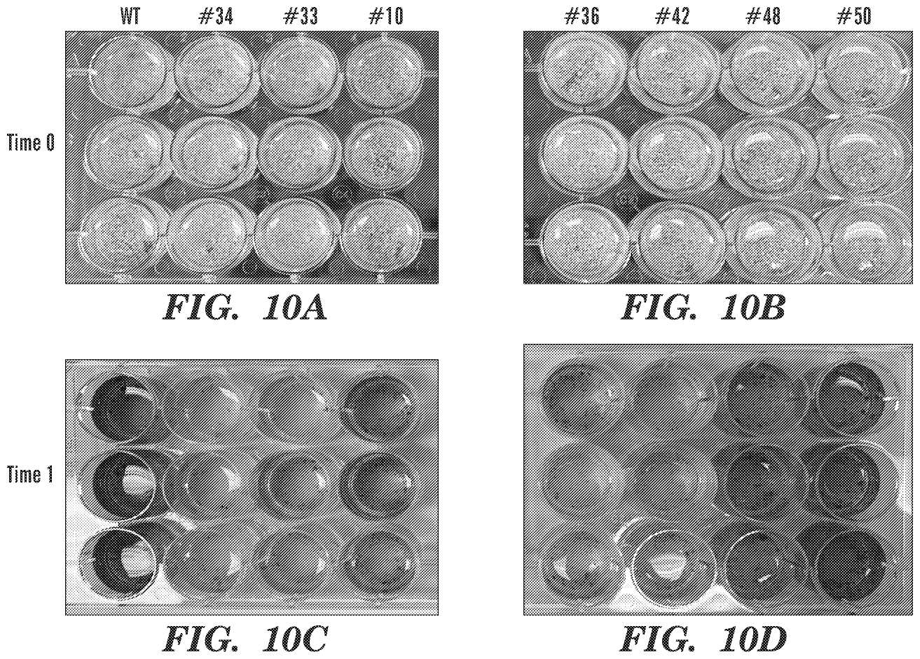

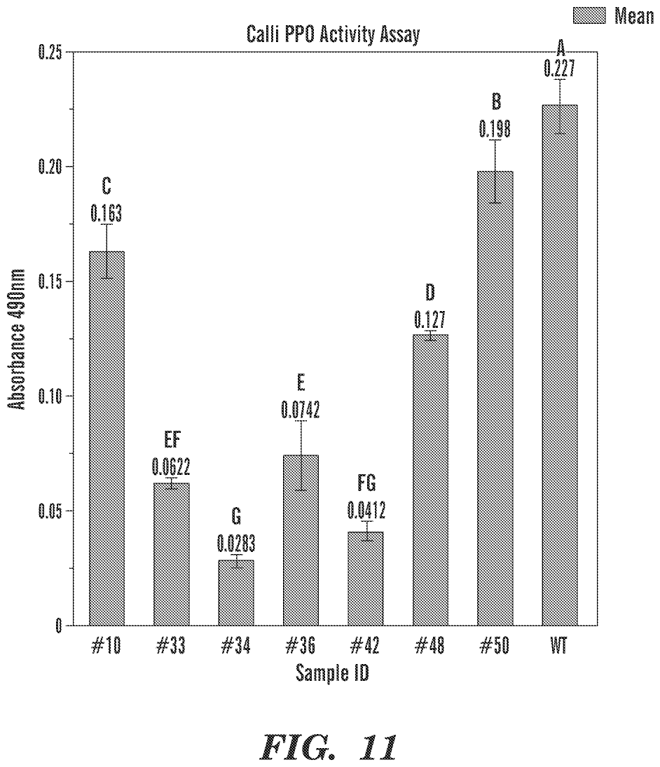

Caffeic acid was used as a substrate to test for PPO activity in the individual callus lines described in Table 5. In brief, equal amounts of calli material (three biological replicates from each line) were submerged in freshly prepared substrate consisting of 6 mM caffeic acid and 25 mM phosphate buffer, pH 7.0, and incubated in the dark at room temperature overnight. Varied discoloration among PPO knockout lines compared to wildtype (non-edited calli) was readily visible (FIGS. 10A-D). Absorbance at 490 nm (A490 nm) was measured with 300 μl of the supernatant in a 96 well plate using a SPECTROstar Nano microplate reader (BMG Labtech) with two technical replicates. The absorbance was measured at both Time 0 and the end Time point (Time 1 as indicated in FIGS. 10C-D). PPO activity was calculated using the absorbance from the end point (Time 1) subtracting the absorbance at time 0 (Jockusch, “The Role of Host Genes, Temperature and Polyphenol Oxidase in the Necrotization of TMV Infected Tobacco Tissue,” J. Phytopathol. Z. 55:185-192 (1966), which is hereby incorporated by reference in its entirety). The PPO activity of each line was graphed and a test for statistical difference was performed with a student t-test (FIG. 11). Lines labeled with different letters were statistically significantly different from each other.

All lines shown in FIG. 11 had statistically significantly less PPO activity than the wildtype avocado. Even though Line 50 had a KO score of only 51%, a significant reduction in PPO was measurable. This result indicated that a heterozygous mutation in PPO-A could affect PPO activity levels. Editing of PPO-A showed a decrease in PPO activity of up to 44% (Line 48). Lines 33, 34, 36, and 42 had editing of both PPO-A and PPO-B genes and showed a decrease in PPO activity of up to 87.5% (Line 34).

### Example 6—Molecular Characterization of Avocado Plants Regenerated from Selected PPO-A and PPO-B Edited Callus Lines

A total of 16 avocado seedlings were germinated from embryos regenerated from the selected edited callus lines using the methods described in Example 2. Plants were amplicon sequenced to confirm the editing genotype. In brief, plant genomic DNA was extracted and targeted regions were amplified using primers listed in Table 5. The PCR products were sequenced using the primers in Table 5 labeled as sequencing primer. The sequencing results were analyzed using ICE (ice.synthego.com) that calculates overall editing efficiency and determines the profiles of all the different types of edits that are present with their relative abundance.

TABLE 5
Exemplary Primer Sequences

| Candidate | | SEQ | | SEQ |
|---|---|---|---|---|
| Gene | Primer for ICE Analysis | ID NO: | Sequencing Primer | ID NO: |
| PPO-A | F: TTCGAACCTTTGATATGGCA | 62 | TTCGAACCTTTGATATGGCA | 64 |
| | R: ATTACCGAAGATTAGCCGAG | 63 | | |
| PPO-B | F: AGAGGCACTGAAAAGTCAAA | 65 | AGAGGCACTGAAAAGTCAAA | 67 |
| | R: TAACTTTCACTCTCACCAGC | 66 | | |

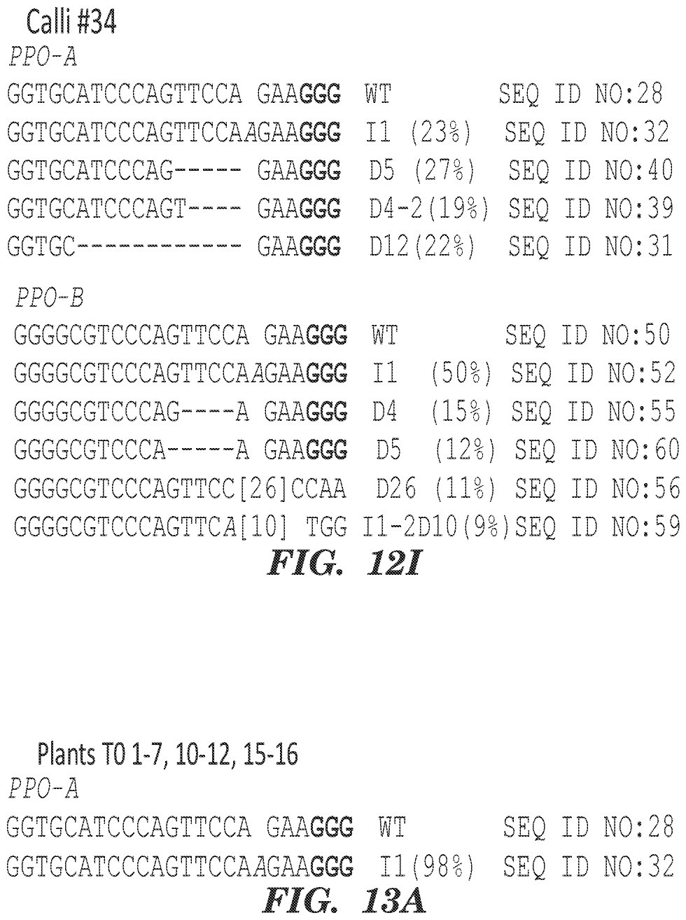

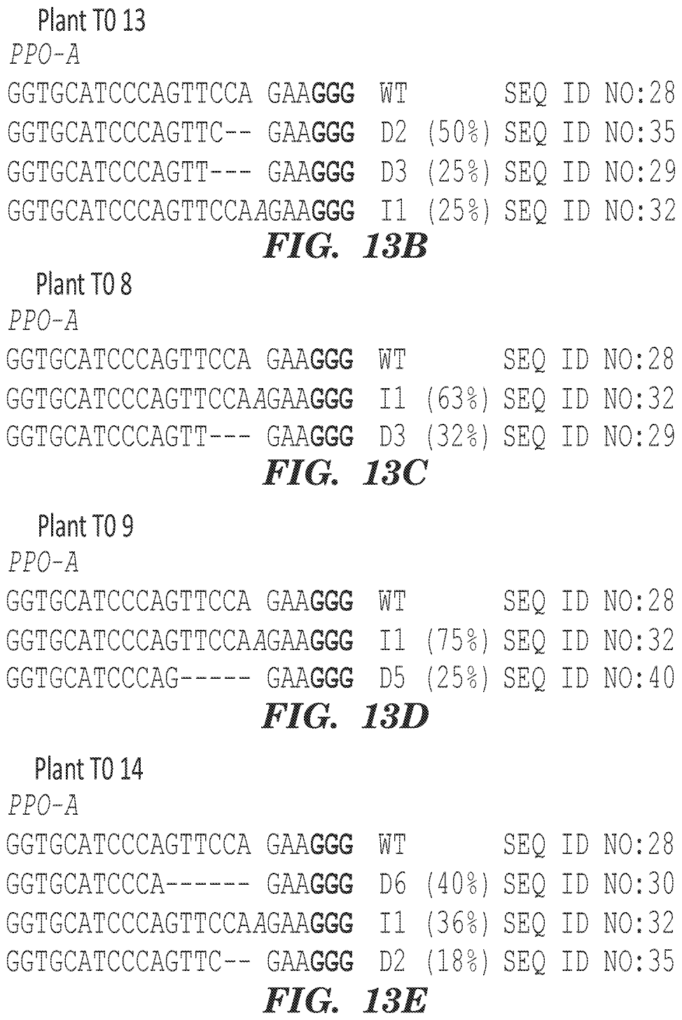

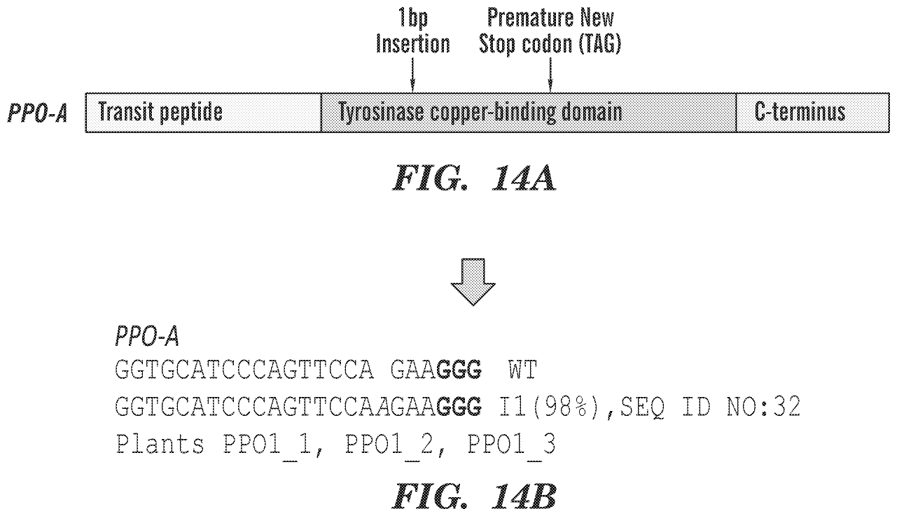

The genome edits of PPO-A from individual plants are described in FIGS. 13A-E. These include the formerly identified mutations in PPO-A called I1 (SEQ ID NO:32), D2 (SEQ ID NO:35), D3 (SEQ ID NO:29), D5 (SEQ ID NO:40), and D6 (SEQ ID NO:30) (Table 4). The PAM site for individual sgRNAs is highlighted in bold font in FIGS. 13A-E. The unedited WT sequence is placed on the top for reference. If the edited allele had more than a 90% frequency in the amplicon sequence, it was considered a homozygous mutant. Twelve of the sixteen plants analyzed (FIG. 13A; TO plants 1, 2, 3, 4, 5, 6, 7, 10, 11, 12, 15, and 16) had the same 1 nt insertion in PPO-A at the same location (SEQ ID NO:32), which resulted in a premature stop codon (FIGS. 14A-B). Considering that the exact same 1 nt insertion was observed in four additional independent events (FIGS. 13A-E), all these 12 plants may not be siblings arising from the same genome editing event. Some plants such as TO-13, TO-14 are chimeric and contain more than two alleles (FIGS. 13A-E). The most frequent mutation of PPO-A was a single nt insertion near the PAM site.

### Example 7—PPO-A Homozygous Knockout Reduces PPO Activity in Leaf Tissue by Up to 68%

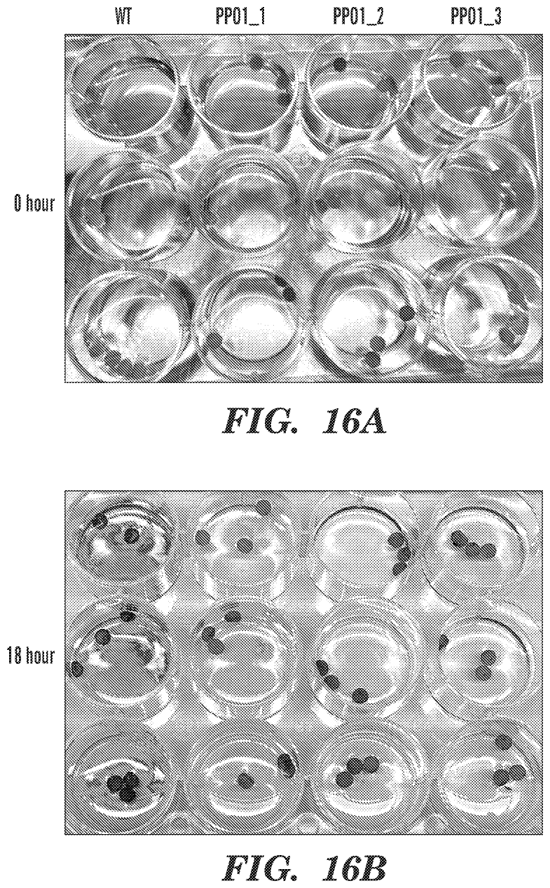

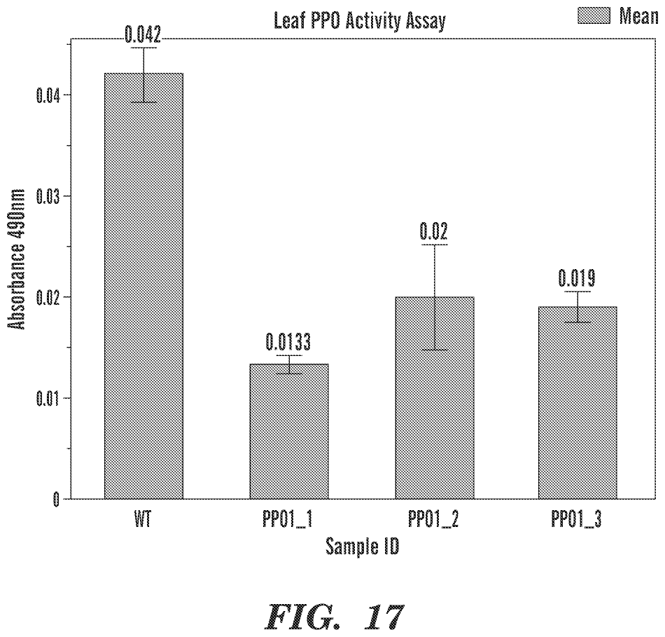

To measure the PPO activity in the regenerated plants from selected callus lines, three avocado plants derived from the same callus with 1 nt insertion of PPO-A were grown to the three-leaf stage to provide enough material for an enzymatic activity assay. These plants were genotyped and confirmed that their PPO-A has 1 nucleotide insertion resulting in a premature stop codon in its tyrosinase copper binding domain (FIGS. 14A-B). Leaves from wild type avocado grown in vitro were sampled as control. Three leaf discs of 3.5 mm in diameter from individual plant were used for the PPO activity assay using caffeic acid as substrate. In brief, 2 ml of freshly prepared substrate (6 mM caffeic acid, 25 mM phosphate buffer, pH 7.0) was added into a 16-well cell culture plate. The leaf discs were added into the solution and incubated in the dark at room temperature overnight. Visible color difference among PPO mutant lines as well as the wildtype (unedited) line was observed (FIGS. 16A-B). Absorbance at 490 nm (A490 nm) was measured with 300 ul of the supernatant in a 96-well plate using a SPECTROstar Nano microplate reader (BMG Labtech) with two technical replicates. The absorbance was measured at both time 0 and the end time point (18 hour as indicated in FIG. 16B). PPO activity was calculated using the absorbance from the end point minus the absorbance at time 0. A test for statistical difference was performed with a student t-test (FIG. 17).

Although preferred embodiments have been depicted and described in detail herein, it will be apparent to those skilled in the relevant art that various modifications, additions, substitutions, and the like can be made without departing from the spirit of the invention and these are therefore considered to be within the scope of the invention as defined in the claims which follow.

What is claimed:

1. An avocado plant cell comprising a loss of function mutation of a nucleic acid sequence encoding a polyphenol oxidase of PPO-A, PPO-B, or PPO-C, wherein the avocado plant cell has reduced polyphenol oxidase activity.

2. The avocado plant cell of claim 1, wherein the cell has a loss of function mutation in both chromosomal alleles of the nucleic acid sequence encoding the polyphenol oxidase.

3. The avocado plant cell of claim 1 or claim 2, wherein the loss of function mutation is in the nucleic acid sequence encoding the polyphenol oxidase of PPO-A.

4. The avocado plant cell of any one of claims 1-3, wherein the loss of function mutation is selected from the mutation contained in the nucleotide sequences selected from the group consisting of SEQ ID NOs:32-48.

5. The avocado plant cell of claim 1 or claim 2, wherein the loss of function mutation is in the nucleic acid sequence encoding the polyphenol oxidase of PPO-B.

6. The avocado plant cell of any one of claim 1, 2, or 5, wherein the loss of function mutation is selected from the mutation contained in the nucleotide sequences selected from the group consisting of SEQ ID NOs:52-60.

7. The avocado plant cell of claim 1 or claim 2 comprising a first loss of function mutation in the nucleic acid sequence encoding the polyphenol oxidase of PPO-A and a second loss of function mutation in the nucleic acid sequence encoding the polyphenol oxidase of PPO-B.

8. The avocado plant cell of claim 7, wherein the first loss of function mutation is selected from the mutation contained in the nucleotide sequences selected from the group consisting of SEQ ID NOs:32-48, and the second loss of function mutation is selected from the mutation contained in the nucleotide sequences selected from the group consisting of SEQ ID NOs:52-60.

9. The avocado plant cell of claim 7 or claim 8, wherein the first loss of function mutation comprises both alleles of the nucleic acid sequence encoding PPO-A.

10. The avocado plant cell of claim 7 or claim 8, wherein the second loss of function mutation comprises both alleles of the nucleic acid sequence encoding PPO-B.

11. The avocado plant cell of any one of claims 7-10, wherein the first loss of function mutation comprises both alleles of the nucleic acid sequence encoding the polyphenol oxidase of PPO-A, and the second loss of function mutation comprises both alleles of the nucleic acid sequence encoding the polyphenol oxidase of PPO-B.

12. The avocado plant cell of any one of claims 7-11 further comprising:
an at least third loss of function mutation of the nucleic acid sequence encoding the polyphenol oxidase selected from the group consisting of PPO-C, PPO-D, PPO-E, PPO-F, PPO-G, and PPO-H.

13. The avocado plant cell of claim 12, wherein the third loss of function mutation comprises both alleles of the nucleic acid sequence encoding a PPO selected from the group consisting of PPO-C, PPO-D, PPO-E, PPO-F, PPO-G, and PPO-H.

14. The avocado plant cell of any one of claims 1-13, wherein the cell is a protoplast.

15. An avocado plant comprising the avocado plant cell of any one of claims 1-14.

16. An avocado fruit comprising the avocado plant cell of any one of claims 1-14.

17. An avocado plant, plant part, or fruit propagated from an avocado plant or fruit of claim 15 or claim 16.

18. The avocado plant, plant part, or fruit of any one of claims 15-17, wherein the plant, plant part, or fruit is free of exogenous DNA.

19. The avocado plant cell, plant, plant part, or fruit of any one of claims 15-18, wherein the polyphenol oxidase activity is reduced by at least 40% compared to the polyphenol oxidase activity of a wild type cell, plant, plant part, or fruit.

20. The avocado plant cell, plant, plant part, or fruit of any one of claims 15-19, wherein the polyphenol oxidase activity is reduced by at least 80% compared to the polyphenol oxidase activity of a wild type cell, plant, plant part, or fruit.

21. A method of making an avocado plant cell comprising a loss of function mutation in polyphenol oxidase A (PPO-A), said method comprising:
isolating nucellar tissue from an avocado plant;
deriving a protoplast cell from the nucellar tissue;
transfecting the protoplast cell with genome editing components;
editing the protoplast cell genome to induce loss of function mutations in polyphenol oxidase A (PPO-A); and
culturing the protoplast cell to make an avocado plant cell comprising a loss of function mutation in polyphenol oxidase A (PPO-A).

22. The method of claim 21 further comprising:
regenerating an avocado plant from the protoplast cell.

23. The method of claim 22, wherein the genome editing components comprise ribonucleoprotein complexes (RNPs) without the use of plant pest sequences.

24. The method of any one of claims 21-23 further comprising:
editing the protoplast cell genome to induce loss of function mutations in polyphenol oxidase B (PPO-B).

25. The method of any one of claims 21-24 further comprising:
editing the protoplast cell genome to induce loss of function mutations in polyphenol oxidase C (PPO-C).

26. A method of making an avocado plant cell comprising altered expression of a gene of choice, said method comprising:
isolating nucellar tissue from an avocado plant;
deriving a protoplast cell from the isolated nucellar tissue;
transfecting the protoplast cell with gene editing components to edit the protoplast cell genome to alter the expression of a gene; and
culturing the protoplast cell to make an avocado plant cell comprising altered expression of the gene.

27. The method of claim 26 further comprising:
regenerating an avocado plant from the protoplast cell.
